(19) United States
(12) Patent Application Publication
(10) Pub. No.: US 2024/0002405 A1
(43) Pub. Date: Jan. 4, 2024

(54) ANTIMICROBIAL COMPOUNDS, SYNTHESIS METHODS, AND USES THEREOF

(71) Applicant: Ankh Life Sciences Limited, Dublin 2 (IE)

(72) Inventors: Rajni Verma, Sterling, KS (US); Krishna Mohan Donavalli, Sterling, KS (US); Gene H. Zaid, Hutchinson, KS (US)

(73) Assignee: Ankh Life Sciences Limited, Dublin 2 (IE)

(21) Appl. No.: 18/327,213

(22) Filed: Jun. 1, 2023

Related U.S. Application Data
(60) Provisional application No. 63/347,752, filed on Jun. 1, 2022.
(60) Provisional application No. 63/405,163, filed on Sep. 9, 2022.

(51) Int. Cl. C07D 519/00; C07D 471/04; A61P 31/10; A61P 31/04

(52) U.S. Cl. CPC C07D 519/00; A61P 31/04; A61P 31/10; C07D 471/04

(57) ABSTRACT

Fused tricyclic compounds and novel methods of synthesizing, using, and/or administering the same. Methods of inhibiting microbial activity and/or treating a microbial infection with fused tricyclic compounds, bicyclic compounds, and other polycyclic compounds. The treatment of subjects suffering from a microbial infection comprises the step of administering to the subject one or more fused tricyclic compounds, bicyclic compounds, and other polycyclic compounds or a composition comprising one or more fused tricyclic compounds, bicyclic compounds, and other polycyclic compounds.

### CROSS-REFERENCE TO RELATED APPLICATIONS

The present application claims the priority benefit of U.S. Provisional Patent Application Ser. No. 63/405,163, filed Sep. 9, 2022, entitled NOVEL COMPOUNDS AND SYNTHESIS METHODS THEREOF, and U.S. Provisional Patent Application Ser. No. 63/347,752, filed Jun. 1, 2022, entitled ANTIMICROBIAL COMPOUNDS AND METHODS OF USE, each of which is incorporated by reference in its entirety herein.

### SEQUENCE LISTING

The following application contains a sequence listing submitted electronically as a Standard ST.26 compliant XML file entitled “SequenceListing_57182US.xml,” created on Jun. 1, 2023, as 7,345 bytes in size, the contents of which are incorporated by reference herein.

### BACKGROUND OF THE INVENTION

### Field of the Invention

The present disclosure is broadly concerned with fused tricyclic compounds and novel methods of synthesizing the same. The present invention also relates to methods of inhibiting microbial activity and/or treating a microbial infection with fused tricyclic compounds, as well as other polycyclic compounds, representing a new class of plant-based antimicrobial compounds.

### Description of Related Art

There are about one trillion species of microorganisms on Earth, and these microorganisms generally fall into one of the following groups: bacteria, archaea, fungi (yeasts and molds), algae, protozoa, and viruses.

Notably, many microorganisms characterized as bacteria, viruses, fungi, and protozoa pose a serious risk to human health. For example, according to the World Health Organization, antibiotic resistant infections currently claim more than 700,000 lives world-wide each year. High rates of resistance are frequently observed worldwide for antibiotics used to treat common infections, including urinary tract infection, sepsis, sexually transmitted infections, and some forms of diarrhea. Antibiotic resistance infections are usually caused by one or more the following pathogens: E. coli, S. aureus, P. aeruginosa, and E. faecalis.

Like bacteria, viruses also pose a serious risk to human health. For example, the COVID-19 pandemic, also known as the coronavirus pandemic, is an ongoing pandemic of coronavirus disease 2019 (COVID-19) caused by severe acute respiratory syndrome coronavirus 2 (SARS-CoV-2), first identified in December 2019 in Wuhan, China. The World Health Organization declared the outbreak a Public Health Emergency of International Concern in January 2020 and a pandemic in March 2020. Within a few months, the virus had spread across the globe causing more than 52.3 million confirmed cases and more than 1.28 million deaths attributed to COVID-19 by November 2020. COVID-19 mainly spreads through the air when people are near each other long enough, primarily via small droplets or aerosols, as an infected person breathes, coughs, sneezes, sings, or speaks. Transmission via fomites (contaminated surfaces) has not been conclusively demonstrated. It can spread as early as two days before infected persons show symptoms (presymptomatic), and from asymptomatic (no symptoms) individuals. People remain infectious for up to ten days in moderate cases, and two weeks in severe cases. Like bacteria and viruses, fungal infections affect more than 1 billion people each year including 150 million cases of severe and life-threatening infections specifically in elderly people, critically ill and immunocompromised patients. Candida albicans is the most common species of Candida which can cause infection, candidiasis. In view of the foregoing, there is accordingly a need in the art for new and effective treatments against microorganisms, particularly bacteria, fungi, and viruses.

### SUMMARY OF THE INVENTION

The major classes of antibiotics currently include penicillins (beta lactams), polypeptides, tetracyclines, and sulfonamides. The present disclosure concerns not only novel compounds but a new family of antibiotic and antimicrobial drugs. These molecules are nature inspired and, in particular, derived from plant-based chemistry. The present disclosure is concerned with fused tricyclic compounds and novel methods of synthesizing the same. The present disclosure is also concerned with methods of inhibiting microbial activity and/or treating microbial infections via introduction, application, contact, and/or administration of fused tricyclic compounds. As used herein, the term “inhibit” refers to a reduction or decreased microbial titer or quantity, compared to a baseline, and the term “microbial activity” refers to the activity, growth, replication, and/or viability of microorganisms, such as bacteria, viruses, protozoa, and fungi. The term “microbial infection” refers to the invasion and multiplication/replication of harmful microorganisms, such as certain bacteria, viruses, protozoa, and fungi, in body tissues, which leads to detectable symptoms of illness and/or disease, such as inflammation, tissue damage, sores, fever/chills, fatigue, pain, swelling, respiratory dysfunction, and the like.

### BRIEF DESCRIPTION OF THE FIGURES

FIG. 1A shows a reaction mechanism of harmaline+aldehyde to a tricyclic β-carboline adduct compound.

FIG. 1B shows a reaction mechanism of harmaline+aldehyde to a tricyclic β-carboline adduct compound to a tricyclic β-carboline dimer compound.

FIG. 2 is an annotated photograph of extraction glassware attached to round bottom flask with activated molecular sieves in cellulose extraction thimble.

FIG. 3 shows a reaction mechanism of GK506.2Im (Example 1).

FIG. 4 show the Synthesis of Tetrahydro Harmine dimer (Top), Harmine dimers (Bottom) from Harmaline dimers (Example 1).

FIG. 5 shows the Harmaline and 4-imidazolecarboxaldehyde Reaction Scheme (Example 2).

FIG. 6 shows the Harmaline and 2-pyridinecarboxaldehyde Reaction Scheme to Yield GK517.2.

FIG. 7 shows the Harmaline and 3-pyridinecarboxaldehyde Reaction Scheme to Yield GK517.3.

FIG. 8 shows the Harmaline and 2-pyridinecarboxaldehyde Reaction Scheme to Yield GK517.4.

FIG. 9 shows the Harmaline and 5-thiazolecarboxaldehyde Reaction Scheme to Yield GK524.

FIG. 10 shows the Harmaline and 6-(Trifluoromethyl)pyridine-3-carboxaldehyde Reaction Scheme to Yield GK585.

FIG. 11 shows the GK Reaction Method Used to Synthesize GZ440/6.

FIG. 12A shows Step 1 of Mechanism to synthesize β-carboline dimers (Example 3).

FIG. 12B shows Step 2 of Mechanism to synthesize β-carboline dimers (Example 3).

FIG. 13 shows cartoon illustration of microbial inoculum by colony suspension as recommended by CLSI guidelines.

FIG. 14 shows cartoon illustration of Broth microdilution assay for antibacterial testing as recommended by CLSI protocol.

FIG. 15 is a graph of E. coli response to GK506.2Im and GK517.3 at 24 hrs. of incubation.

FIG. 16 is a graph of P. aeruginosa response to GK506.2Im and GK517.3 at 24 hrs. of incubation.

FIG. 17 is a graph of S. aureus response to GK506.2Im and GK517.3 at 10 hrs. of incubation.

FIG. 18 is a graph of S. aureus response to GK506.2Im and GK517.3 at 22 hrs. of incubation.

FIG. 19 is a graph of S. aureus Dose response curve for GK506.2Im and GK517.3 at 22 hrs. of incubation.

FIG. 20 is a graph of S. aureus response to GK506.2Im and GK517.3 molecules vs DMSO control.

FIG. 21 is a graph of S. aureus response to GK506.2Im and GK517.3 at 36 hrs. of incubation.

FIG. 22 is a graph of S. aureus response to GK506.2Im and GK517.3 at 57 hrs. of incubation.

FIG. 23 is a graph of S. aureus response to GK506.2Im and GK517.3 at 81 hrs. of incubation.

FIG. 24 is a graph of S. aureus response to GK506.2Im and GK517.3 at 90 hrs. of incubation.

FIG. 25 is a graph of E. faecalis response to GK506.2Im and GK517.3 at 10 hrs. of incubation.

FIG. 26 is a graph of E. faecalis response to GK506.2Im and GK517.3 at 22 hrs. of incubation.

FIG. 27 is a graph of the E. faecalis Dose response curve of GK506.2Im and GK517.3 at 22 hrs. of incubation.

FIG. 28 is a graph of E. faecalis response to GK506.2Im and GK517.3 molecules vs DMSO.

FIG. 29 is a graph of E. faecalis response to GK506.2Im and GK517.3 at 36 hrs. of incubation.

FIG. 30 is a graph of E. faecalis response to GK506.2Im and GK517.3 at 57 hrs. of incubation.

FIG. 31 is a graph of E. faecalis response to GK506.2Im and GK517.3 at 81 hrs. of incubation.

FIG. 32 is a graph of E. faecalis response to GK506.2Im and GK517.3 at 90 hrs. of incubation.

FIG. 33 is a graph of E. coli and P. aeruginosa response to Harmaline at 24 hrs. of incubation.

FIG. 34 is a graph of E. coli and S. aureus response to Harmaline at 24 hrs. of incubation.

FIG. 35 is a graph of S. aureus and E. faecalis response to Harmaline at 24 hrs. of incubation.

FIG. 36 is a graph of the Dose response curve of gram-positive bacteria, S. aureus and E. faecalis at 24 hrs. of incubation with Harmaline.

FIG. 37 is a graph of S. aureus and E. faecalis response to GZ440/6 at 24 hrs. of incubation.

FIG. 38 is a graph of E. coli and P. aeruginosa response to GZ440/6 at 24 hrs. of incubation.

FIG. 39 is a graph of the Dose response curve of gram-positive bacteria, S. aureus and E. faecalis at 24 hrs. of incubation with GZ440/6.

FIG. 40 is a graph of MRSA response to GZ440/6, GK506.2Im and GK517.3 at 24 hrs. of incubation.

FIG. 41 is a graph of MRSA response to GZ440/6, GK506.2Im and GK517.3 at 40 hrs. incubation.

FIG. 42 is a graph of MRSA response to GZ440/6, GK506.2Im and GK517.3 at 63 hrs. incubation.

FIG. 43 is a graph of MRSA response to GZ440/6, GK506.2Im and GK517.3 at 90 hrs. incubation.

FIG. 44 is a graph of the Dose response curve of MRSA at 24 hrs. of incubation with GZ440/6, GK506.2Im and GK517.3.

FIG. 45 is a graph of VREF response to GZ440/6, GK506.2Im and GK517.3 at 24 hrs. incubation.

FIG. 46 is a graph of VREF response to GZ440/6, GK506.2Im and GK517.3 at 40 hrs. incubation.

FIG. 47 is a graph of VREF response to GZ440/6, GK506.2Im and GK517.3 at 63 hrs. incubation.

FIG. 48 is a graph of VREF response to GZ440/6, GK506.2Im and GK517.3 at 90 hrs. incubation.

FIG. 49 is a graph of the Dose response curve of VREF at 24 hrs. of incubation with GZ440/6, GK506.2Im and GK517.3.

FIG. 50 shows photographs of culture plates for MBC data of a) GK517.3, b) GK506.2 and c) GZ440/6 against MRSA.

FIG. 51 shows photographs of 96-well plates and culture plates from: (a) MIC studies of GK506.2Im (A-C) and GK517.3 (D-F) against S. aureus; (b) Top-MIC studies of GK506.2Im (E-G), Bottom-MIC studies of GK517.3 (E-G); (c) Top 2 plates-MBC studies of GK506.2Im, Bottom 2 plates-MBC studies of GK517.3 against S. aureus; (d) Top 2 plates-MBC studies of GK506.2Im, Bottom 2 plates-MBC studies of GK517.3 against E. faecalis.

FIG. 52 is a graph of E. coli response to GK524 and GK585 after 24 hours of incubation.

FIG. 53 is a graph of E. coli response to GK524 and GK585 after 48 hours of incubation.

FIG. 54 is a graph of S. aureus response to GK524 and GK585 at 24 hours of incubation.

FIG. 55 is a graph of S. aureus response to GK524 and GK585 after 48 hours of incubation.

FIG. 56 is a graph of MRSA response to GK524 and GK585 at 24 hrs. incubation.

FIG. 57 is a graph of MRSA response to GK524 and GK585 after 48 hours of incubation.

FIG. 58 is a graph of E. coli response to GK517.2 and GK517.4 after 24 hours of incubation.

FIG. 59 is a graph of E. coli response to GK517.2 and GK517.4 after 48 hours of incubation.

FIG. 60 is a graph of S. aureus response to GK517.2 and GK517.4 after 24 hours of incubation.

FIG. 61 is a graph of S. aureus response to GK517.2 and GK517.4 after 48 hours of incubation.

FIG. 62 is a graph of MRSA response to GK517.2 and GK517.4 after 24 hours of incubation.

FIG. 63 is a graph of MRSA response to GK517.2 and GK517.4 after 48 hours of incubation.

FIG. 64A is a graph of the results from Cytotoxicity Run 1 with GZ440-6 in Example 6.

FIG. 64B is a graph of the results from Cytotoxicity Run 2 with GZ440-6 in Example 6.

FIG. 64C is a graph of the results from Cytotoxicity Run 3 with GZ440-6 in Example 6.

FIG. 64D is a graph of the Cytotoxicity Average with GZ440-6 in Example 6.

FIG. 65A is a graph of the results of the virus plaque formation assay using GZ440-6 at different concentrations from Example 6.

FIG. 65B is a graph of the results of the virus plaque formation assay using GZ440-6 at different concentrations from Example 6.

FIG. 65C is a graph of the results of the virus plaque formation assay using GZ440-6 at different concentrations from Example 6.

FIG. 65D is a graph of the results of the virus plaque formation assay using GZ440-6 at different concentrations from Example 6.

FIG. 66A is a graph of the results from quantitation of viral copy number when treating SARS-CoV2 with GZ440-6 at different concentrations.

FIG. 66B is a graph of the results from quantitation of viral copy number when treating SARS-CoV2 with GZ440-6 at different concentrations.

FIG. 66C is a graph of the results from quantitation of viral copy number when treating SARS-CoV2 with GZ440-6 at different concentrations.

FIG. 67 is a graph of UMU response data for compounds GZ440/6, GK506.2Im, GK524 and GK517.3. UMU IR (induction ratio) is an internationally accepted measure of genotoxicity. The positive control (4-NQC) has the UMU IR value of 7.99.

FIG. 68 is a graph of UMU genotoxicity activity of GK506.2Im, GK517.3 and GK524 without S9 activation. GK517.3 and GK524 are not genotoxic up to 100 μM. GK506.2Im shows toxicity above 85 μM.

FIG. 69 is a graph of UMU genotoxicity activity of GK506.2Im, GK517.3 and GK524 with S9 activation-rat liver enzyme. Metabolites of GK517.3 and GK524 are genotoxic above 65 μM. GK506.2Im metabolites shows genotoxicity above 50 μM.

FIG. 70 shows the Reaction scheme of GK580.

FIG. 71 shows the Reaction scheme of GK580.HCl.

FIG. 72A shows the Reaction scheme of GK578.

FIG. 72B shows the Reaction mechanism for synthesizing GK578 through intermediate compounds GK414, GK395, and GK596.

FIG. 73A is a graph of S. aureus response to different concentrations of GK580 (bar 1) and GK580.HCl (bar 2), DMSO (bar 3) and growth control (bar 4).

FIG. 73B is a graph of MRSA response to different concentrations of GK580 (bar 1) and GK580.HCl (bar 2), DMSO (bar 3) and growth control (bar 4).

FIG. 73C is a graph of E. faecalis response to different concentrations of GK580 (bar 1) and GK580.HCl (bar 2), DMSO (bar 3) and growth control (bar 4).

FIG. 73D is a graph of VREF response to different concentrations of GK580 (bar 1) and GK580.HCl (bar 2), DMSO (bar 3) and growth control (bar 4).

FIG. 74 is a graph of UMU response data for compounds GK580, GK580.HCl and GK660. UMU IR (induction ratio) is an internationally accepted measure of genotoxicity. The positive control (4-NQC) has the UMU IR value of 7.99.

FIG. 75 is a graph of GK578 and its salt activity on E. coli at different concentrations.

FIG. 76A shows the original synthesis scheme of converting GK578 into GK508 using Grignard reaction.

FIG. 76B shows the original synthesis scheme of GK360 through intermediate GK395.

FIG. 77 shows the general reaction scheme of Harmaline and 2-Aminobenzaldehydes.

FIG. 78 shows the general reaction scheme of GK366.

FIG. 79 shows the reaction scheme for GK359.

FIG. 80 shows the proposed reaction scheme of GK344.

FIG. 81 shows the reaction scheme for GK375.

FIG. 82 shows the reaction scheme for ester hydrolysis of GK375 to yield GK360.

FIG. 83 shows the reaction scheme for GK431.

FIG. 84 shows the reaction scheme for GK560B and GK346B.

FIG. 85 shows the reaction scheme for GK346A.

FIG. 86 shows the reaction scheme for GK600.

FIG. 87 shows a graph of the Antifungal activity of GK580, GK524, GK360 and GK395 after 24 hours of incubation.

FIG. 88 shows a graph of the Antifungal activity of GK580, GK524, GK360 and GK395 after 48 hours of incubation.

FIG. 89A shows a graph of GK600 activity on E. coli after 10 hours incubation.

FIG. 89B shows a graph of GK580 and GK600 activity on E. coli.

FIG. 90 shows a graph of GK600 activity on S. aureus after 16 hours incubation.

FIG. 91 shows a graph of GK600 activity on MRSA after 20 hours incubation.

FIG. 92 shows a graph of GK317 activity on E. coli at 8 hrs.

FIG. 93 shows a graph of GK317 activity on S. aureus at 16 hrs.

FIG. 94 shows a graph of GK350 activity on E. coli at 8 hrs.

FIG. 95 shows a graph of GK350 activity on S. aureus at 16 hrs.

FIG. 96 shows a graph of GK360 activity on E. coli at 8 hrs.

FIG. 97 shows a graph of GK360 activity on S. aureus at 16 hrs.

FIG. 98 shows a graph of GK395 and GK395 salts activity on E. coli at 8 hrs.

FIG. 99 shows a graph of GK395 activity on S. aureus at 16 hrs.

FIG. 100A is a graph of GK599 activity on S. aureus.

FIG. 100B is a graph of GK599.HCl, GK599.NaOH and GK480 activity data on S. aureus.

FIG. 101 is a graph of GK599.HCl, GK599.NaOH activity data on MRSA.

FIG. 102A is a graph of GK599 activity on E. coli.

FIG. 102B is a graph of GK599.HCl, GK599.NaOH and GK480 activity on E. coli.

### DETAILED DESCRIPTION

### Compounds

### 1. Tricyclic β-Carboline Adducts

Several fused tricyclic compounds described herein are effective inhibitors of microbial activity and/or microbial infections. These compounds are advantageously derived from plant-based chemistries. In one or more embodiments, the fused tricyclic compounds may be tricyclic β-carboline adducts, preferably the tricyclic β-carboline adducts shown below:

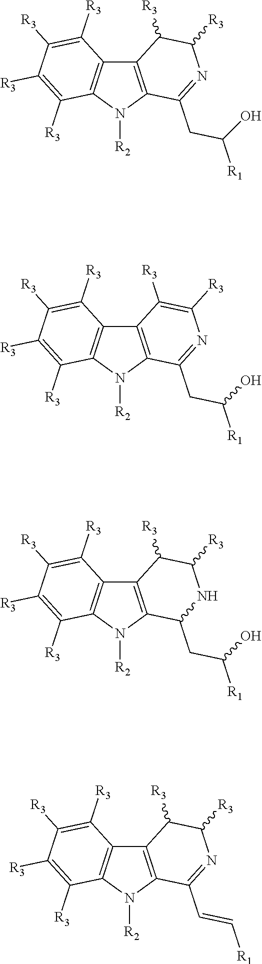

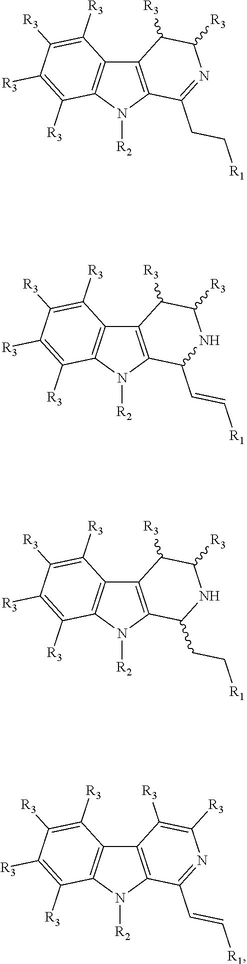

where each R₁, R₂, and R₃ is independently selected from the group consisting of possible substituents defined in the table below:

| Group | Possible substituents |
|---|---|
| R₁ | Substituted or unsubstituted Aromatic, Antiaromatic, or non-aromatic compounds |
| | up to 10 atoms, for example, Furan, imidazole, Benzene, Pyridine, Indole, Indazole, |
| | Cyclooctatetraene, [10]annulene (Cyclodecapentaene), Pentalene, Indene, |
| | Naphthalene, Heptalene, Biphenylene, as-indacene, acenaphthylene, fluorene, |
| | phenalene, Anthracene, Pyrene, Fluoranthene, Imidazopyridines, Pyrazopyridines, |
| | Oxazolopyridines, Isooxazolopyridines, Cyclopropane, cyclopentane, methyl, |
| | ethyl, propyl, isopropyl, pentadiene, hexane, or hexene, any of which may or may |
| | not be substituted such as with halogens, alkyls (C1-C15), hydroxy, H-donor groups |
| | (e.g., carboxylic acids, amines, and amides), H-bond acceptor groups (e.g., ethers, |
| | esters, aldehydes, or ketones). Fused polycyclic (bi-, tri-, tetra-, etc.) and |
| | heterocyclic rings, such as thioridazine, purines, coumarins, indoles, or indazole, |
| | with substitutions at one or more carbons of the rings. |
| R₂ | Hydrogen, halogens, sulfonyl chloride, Sulphonic acid, Sulfonamide, thiol, |
| | hydroxy, Alkoxy (C1-C15), poly methoxy (C2-C15), H-donor groups (e.g., |
| | carboxylic acids, amines, and amides), H-bond acceptor groups (e.g., ethers, esters, |
| | aldehydes, or ketones), Aromatic, Antiaromatic, or non-aromatic compounds up to |
| | 10 atoms, for example, Furan, imidazole, Benzene, Pyridine, Indole, Indazole, |
| | Cyclooctatetraene, [10]annulene, Pentalene, Indene, Naphthalene, Heptalene, |
| | Biphenylene, as-indacene, acenaphthylene, fluorene, phenalene, Anthracene, |
| | Pyrene, Fluoranthene, Imidazopyridines, Pyrazopyridines, Oxazolopyridines, |
| | Isooxazolopyridines, Cyclopropane, cyclopentane, methyl, ethyl, propyl, isopropyl, |
| | pentadiene, hexane, or hexene, any of which may or may not be substituted such as |
| | with halogens, alkyls (C1-C15), hydroxy, H-donor groups (e.g., carboxylic acids, |
| | amines, and amides), H-bond acceptor groups (e.g., ethers, esters, aldehydes, or |
| | ketones). Fused polycyclic and heterocyclic rings, such as thioridazine, purines, |
| | coumarins, indoles, or indazole, with substitutions at one or more carbons of the |
| | rings. |
| R₃ | Hydrogen, Oxygen, halogens, sulfonyl chloride, Sulphonic acid, thiols, alkyls (C1- |
| | C15), Nitro groups, hydroxy, Alkoxy (C1-C15), H-donor groups (e.g., carboxylic |
| | acids, amines, and amides), H-bond acceptor groups (e.g., ethers, esters, aldehydes, |
| | or ketones), Aromatic, Antiaromatic, or non-aromatic compounds up to 10 atoms, |
| | for example, Furan, imidazole, Benzene, Pyridine, Indole, Indazole, |
| | Cyclooctatetraene, [10]annulene, Pentalene, Indene, Naphthalene, Heptalene, |
| | Biphenylene, as-indacene, acenaphthylene, fluorene, phenalene, Anthracene, |
| | Pyrene, Fluoranthene, Imidazopyridines, Pyrazopyridines, Oxazolopyridines, |
| | Isooxazolopyridines, Cyclopropane, cyclopentane, methyl, ethyl, propyl, isopropyl, |
| | pentadiene, hexane, or hexene, any of which may or may not be substituted such as |
| | with halogens, alkyls (C1-C15), hydroxy, H-donor groups (e.g., carboxylic acids, |
| | amines, and amides), H-bond acceptor groups (e.g., ethers, esters, aldehydes, or |
| | ketones). Fused polycyclic and heterocyclic rings, such as thioridazine, purines, |
| | coumarins, indoles, or indazole, with substitutions at one or more carbons of the |
| | rings. |

In one or more embodiments, other harmaline, harmine, and tetrahydroharmine derivatives of tricyclic β-carboline adduct compounds are furnished from starting compounds using oxidizing or reducing agents, as shown in the table below.

| | |
|---|---|
| Starting | Resulting Derivative |

Oxidizing Agent−−>

| | |
|---|---|
| 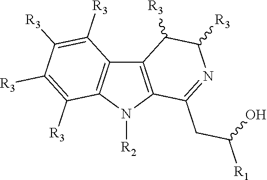 | 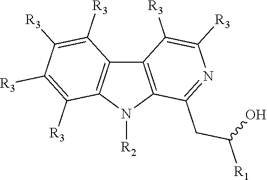 |
| 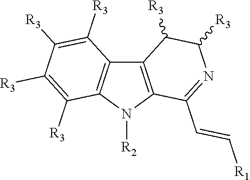 | 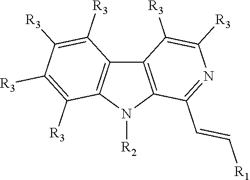 |

Reducing Agent−−>

| | |
|---|---|
| 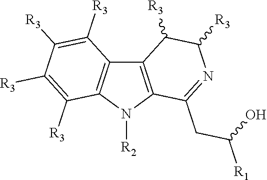 | 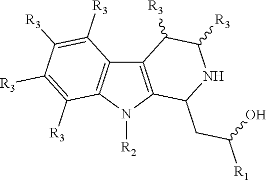 |
| 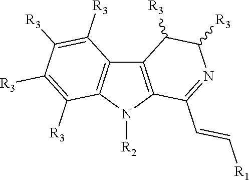 | 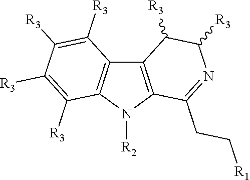 |
| 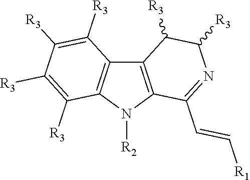 | 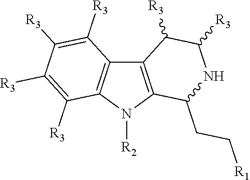 |
| 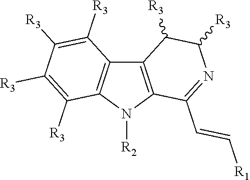 | 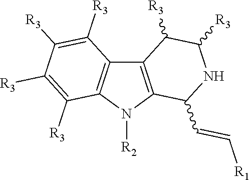 |

In one or more preferred embodiments, the fused tricyclic β-carboline compounds are selected from the group of one or more of the following compounds or variants thereof as defined herein:

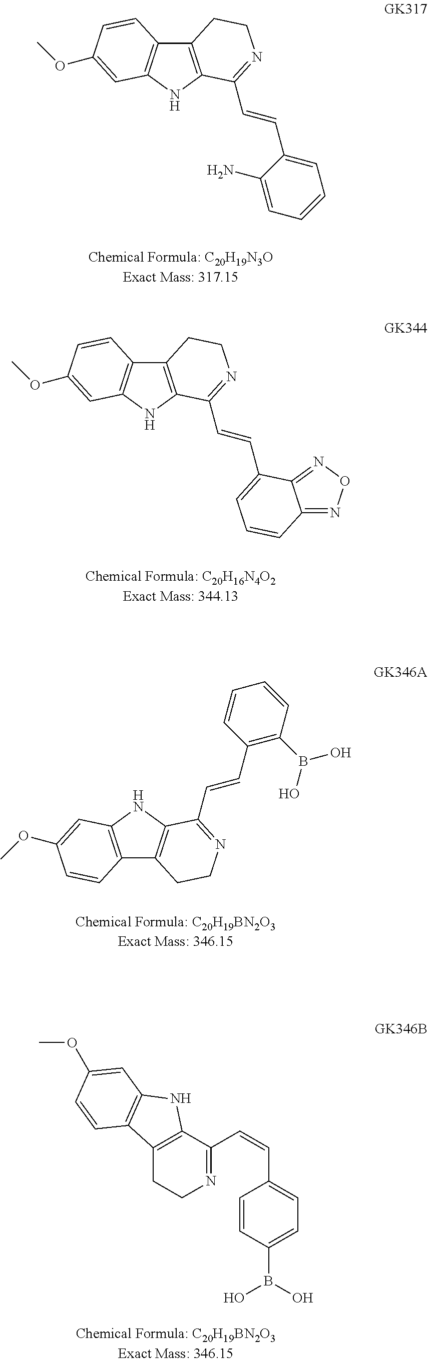 [SMILES: COc1ccc2c3c([nH]c2c1)C(/C=C/c1ccccc1N)=NCC3 ; COc1ccc2c3c([nH]c2c1)C(/C=C/c1cccc2nonc12)=NCC3 ; COc1ccc2c3c([nH]c2c1)C(/C=C/c1ccccc1B(O)O)=NCC3 ; COc1ccc2c3c([nH]c2c1)C(/C=C\c1ccc(B(O)O)cc1)=NCC3]

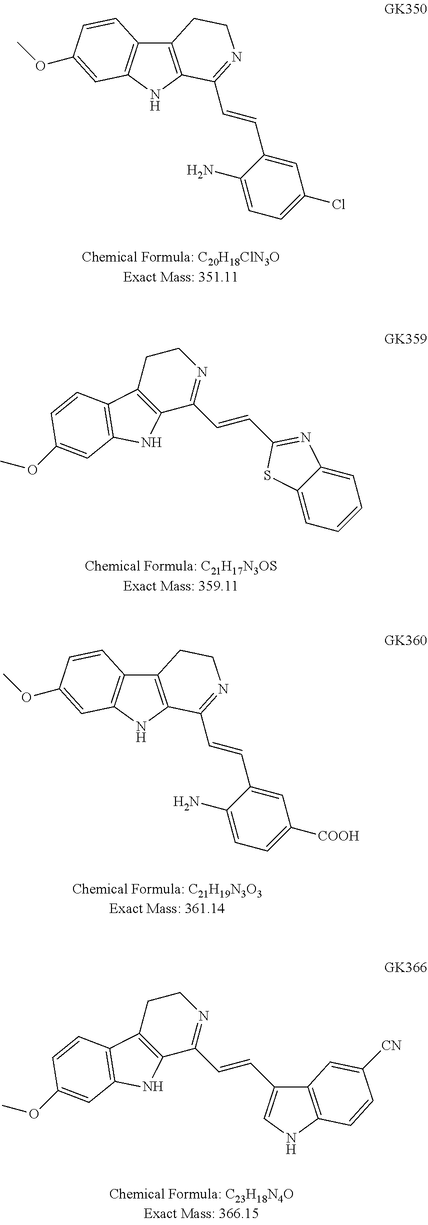 [SMILES: COc1ccc2c3c([nH]c2c1)C(/C=C/c1cc(Cl)ccc1N)=NCC3 ; COc1ccc2c3c([nH]c2c1)C(/C=C/c1nc2ccccc2s1)=NCC3 ; COc1ccc2c3c([nH]c2c1)C(/C=C/c1cc(C(=O)O)ccc1N)=NCC3 ; COc1ccc2c3c([nH]c2c1)C(/C=C/c1c[nH]c2ccc(C#N)cc12)=NCC3]

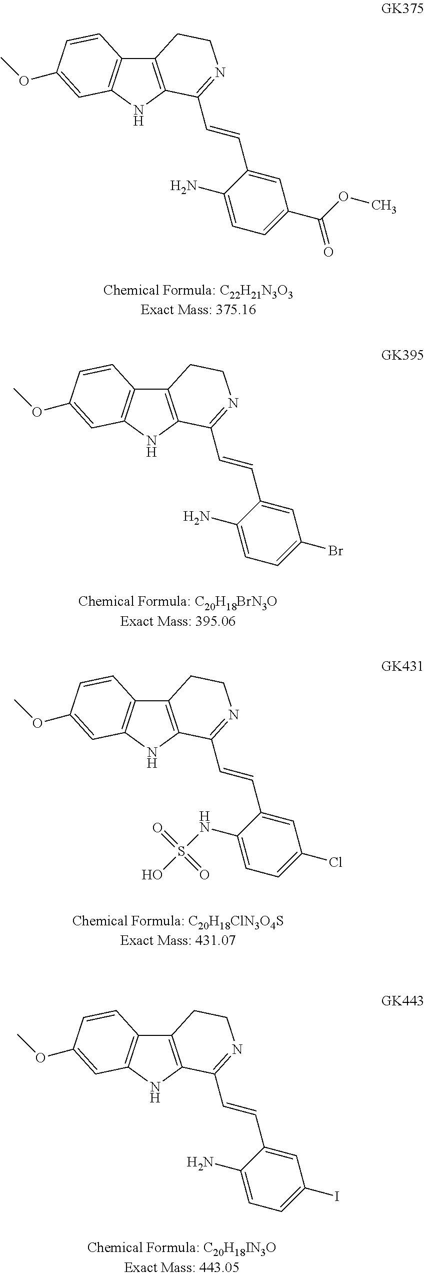

Particularly preferred tricyclic β-carboline adducts are selected from:

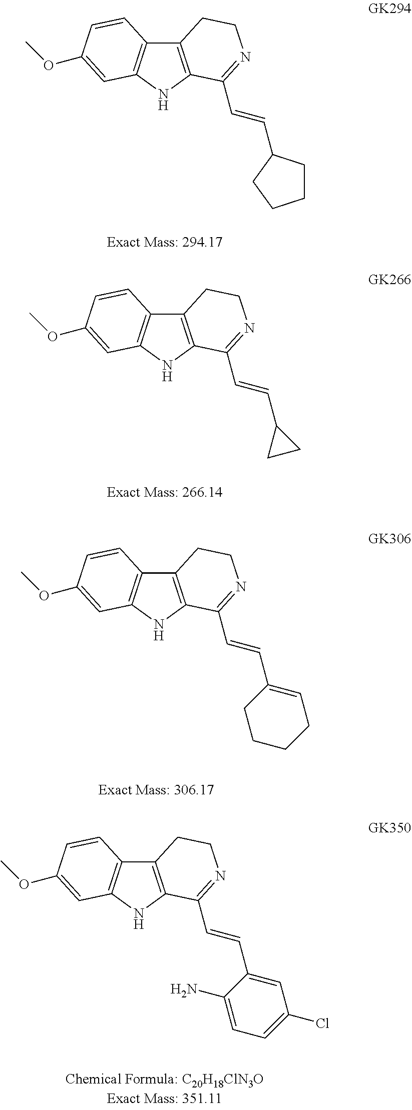 [SMILES: COc1ccc2c3c([nH]c2c1)C(/C=C/C1CCCC1)=NCC3 ; COc1ccc2c3c([nH]c2c1)C(/C=C/C1CC1)=NCC3 ; COc1ccc2c3c([nH]c2c1)C(/C=C/C1=CCCCC1)=NCC3 ; COc1ccc2c3c([nH]c2c1)C(/C=C/c1cc(Cl)ccc1N)=NCC3]

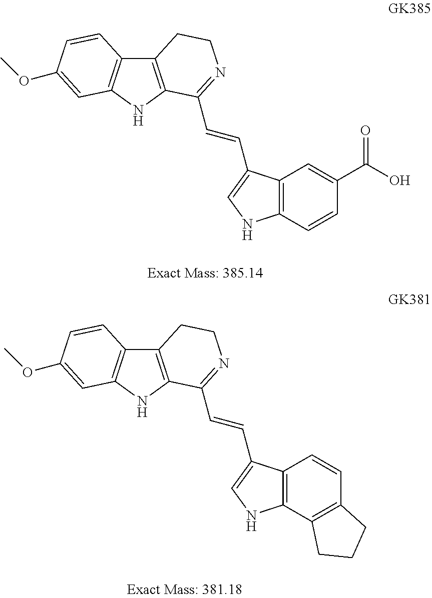 [SMILES: COc1ccc2c3c([nH]c2c1)C(/C=C/c1c[nH]c2ccc(C(=O)O)cc12)=NCC3 ; COc1ccc2c3c([nH]c2c1)C(/C=C/c1c[nH]c2c4c(ccc12)CCC4)=NCC3]

### 2. Tricyclic β-Carboline Dimer Compounds

In one or more embodiments, the fused tricyclic compounds are tricyclic β-carboline dimer compounds comprising two β-carboline moieties linked via a CH₂ methine group bonded to respective methyl substituents of the β-carboline moieties. In these preferred embodiments, the β-carboline moieties are preferably harmine, harmaline, and tetrahydro harmine moieties, as shown below (a) harmine, b) harmaline, and c) tetrahydro harmine):

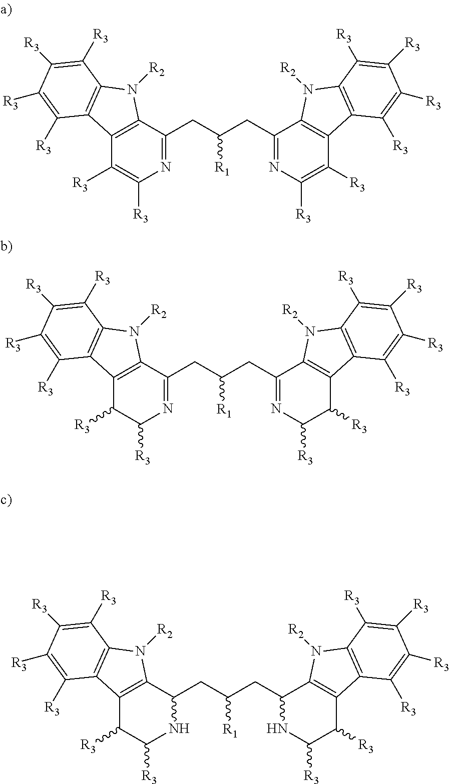

here each R₁, R₂, and R₃ is individually selected from the options defined in the table below.

| Group | Possible substitutions |
|---|---|
| R₁ | Aromatic, Antiaromatic, or non-aromatic compounds up to 10 atoms; for example, |
| | Furan, imidazole, Benzene, Pyridine, Indole, Indazole, Cyclooctatetraene, |
| | [10]annulene, Pentalene, Indene, Naphthalene, Heptalene, Biphenylene, as- |
| | indacene, acenaphthylene, fluorene, phenalene, Anthracene, Pyrene, Fluoranthene, |
| | Imidazopyridines, Pyrazopyridines, Oxazolopyridines, Isooxazolopyridines, |
| | Cyclopropane, cyclopentane, methyl, ethyl, propyl, isopropyl, pentadiene, hexane, |
| | or hexene, any of which may or may not be substituted such as with halogens, |
| | alkyls (C1-C15), hydroxy, H-donor groups (e.g., carboxylic acids, amines, and |
| | amides), or H-bond acceptor groups (e.g., ethers, esters, aldehydes, or ketones). |
| | Fused heterocyclic rings, such as thioridazine, purines, coumarins, indoles, or |
| | indazole, with substitutions at one or more carbons of the rings. |
| R₂ | Hydrogen, halogens, sulfonyl chloride, Sulphonic acid, Sulfonamide, thiol, hydroxy, |
| | Alkoxy (C1-C15), poly methoxy (C2-C15), H-donor groups (e.g., carboxylic acids, |
| | amines, and amides), H-bond acceptor groups (e.g., ethers, esters, aldehydes, or |
| | ketones), Substituted or unsubstituted Aromatic, Antiaromatic, or non-aromatic |
| | compounds up to 10 atoms; for example, Furan, imidazole, Benzene, Pyridine, |
| | Indole, Indazole, Cyclooctatetraene, [10]annulene, Pentalene, Indene, Naphthalene, |
| | Heptalene, Biphenylene, as-indacene, acenaphthylene, fluorene, phenalene, |
| | Anthracene, Pyrene, Fluoranthene, Imidazopyridines, Pyrazopyridines, |
| | Oxazolopyridines, Isooxazolopyridines, Cyclopropane, cyclopentane, methyl, |
| | ethyl, propyl, isopropyl, pentadiene, hexane, or hexene, any of which may or may |
| | not be substituted such as with halogens, alkyls (C1-C15), hydroxy, H-donor groups |
| | (e.g., carboxylic acids, amines, and amides), or H-bond acceptor groups (e.g., |
| | ethers, esters, aldehydes, or ketones). Fused heterocyclic rings, such as thioridazine, |
| | purines, coumarins, indoles, or indazole, with substitutions at one or more carbons |
| | of the rings. |
| R₃ | Hydrogen, Oxygen, halogens, Sulfonyl chloride, Sulphonic acid, , thiol, alkyls (C1- |
| | C15), Nitro groups, hydroxy, Alkoxy (C1-C15), H-donor groups (e.g., carboxylic |
| | acids, amines, and amides), H-bond acceptor groups (e.g., ethers, esters, aldehydes, |
| | or ketones), Aromatic, Antiaromatic, or non-aromatic compounds up to 10 atoms; |
| | for example, Furan, imidazole, Benzene, Pyridine, Indole, Indazole, |
| | Cyclooctatetraene, [10]annulene, Pentalene, Indene, Naphthalene, Heptalene, |
| | Biphenylene, as-indacene, acenaphthylene, fluorene, phenalene, Anthracene, |
| | Pyrene, Fluoranthene, Imidazopyridines, Pyrazopyridines, Oxazolopyridines, |
| | Isooxazolopyridines, Cyclopropane, cyclopentane, methyl, ethyl, propyl, isopropyl, |
| | pentadiene, hexane, or hexene, any of which may or may not be substituted such as |
| | with halogens, alkyls (C1-C15), hydroxy, H-donor groups (e.g., carboxylic acids, |
| | amines, and amides), H-bond acceptor groups (e.g., ethers, esters, aldehydes, or |
| | ketones). Fused heterocyclic rings, such as thioridazine, purines, coumarins, |
| | indoles, or indazole, with substitutions at one or more carbons of the rings. |

Other exemplary tricyclic β-carboline dimer compounds can be found in U.S. Publication No. 2022/0033417, filed Dec. 1, 2020, which is incorporated, in its entirety, by reference herein. Advantageously, the structures a), b), and c) differ (from each other) by the number of double bonds each possesses: the more double bonds make the structure rigid and less double bonds make the structure more flexible, which can affect the bioactivity of the structures. In one or more embodiments, the fused tricyclic compounds may comprise tricyclic β-carboline monomers, such as harmaline.

In most preferred embodiments, the tricyclic β-carboline dimer compounds are selected from the group of one or more of the following compounds or variants thereof as defined herein:

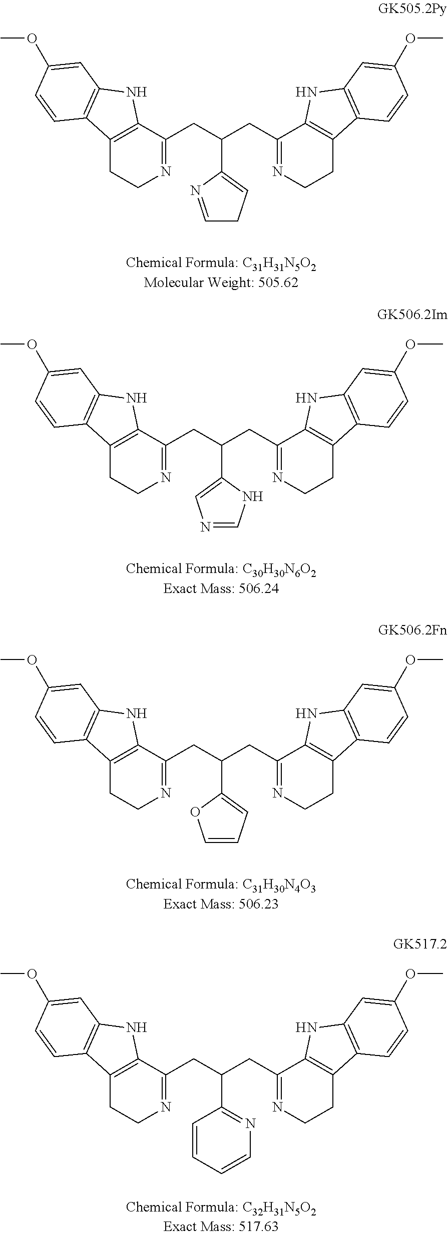

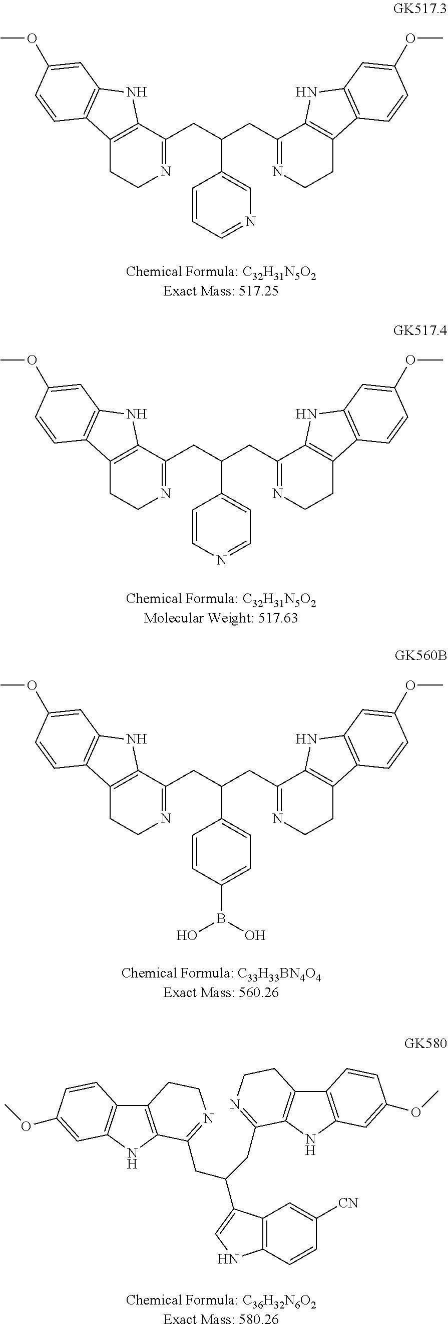

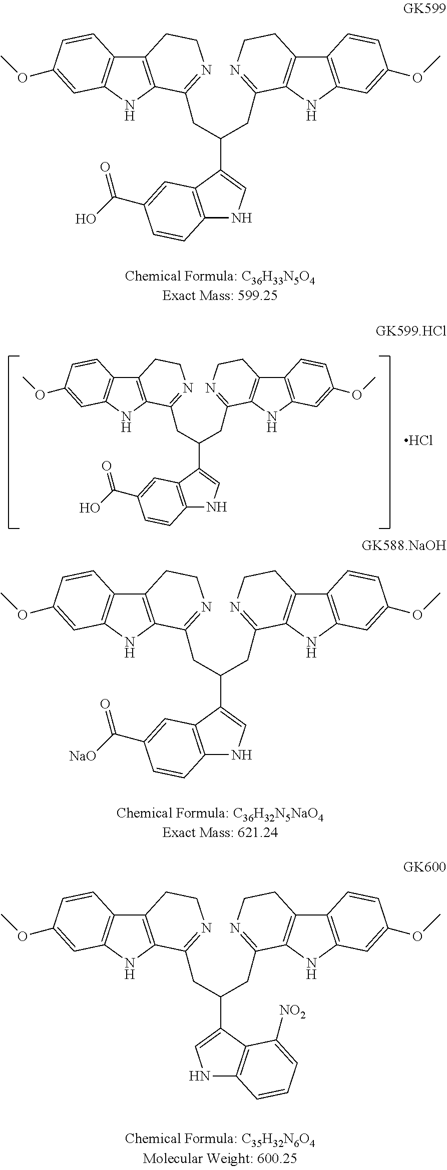

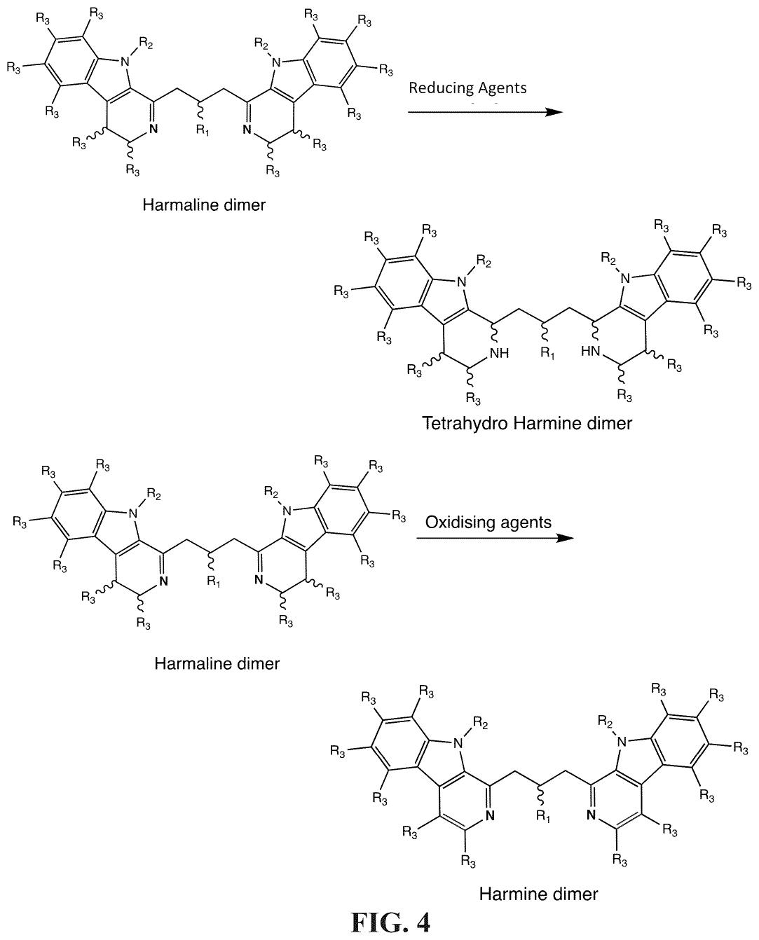

In other embodiments, the tricyclic β-carboline dimer compounds comprising two harmine or tetrahydro harmine moieties may be synthesized using either reducing agents or oxidizing agents, as shown in FIG. 4.

Advantageously, GK505.2Py, GK506.2Fn, GK506.2Im, GK517.2, GK517.3, GK517.4, GK524, GK580, GK580.HCl and GK585 all differ from the tricyclic β-carboline dimer compounds disclosed in U.S. Publication No. 2022/0033417. Particularly, GK505.2Py, GK506.2Fn, GK506.2Im, GK517.2, GK517.3, GK517.4, GK524, GK580 and GK585 each contain a heterocyclic aromatic ring (e.g., pyridine, imidazole, indole etc.) at the tether, while the tricyclic β-carboline dimer compounds disclosed in U.S. Publication No. 2022/0033417 each contain a non-heterocyclic aromatic ring (e.g., benzene) at the tether. The addition of a nitrogen, sulfur, and/or oxygen atom on the heterocyclic ring results in notable physical, chemical, and biological property differences. For example, nitrogen (as well as sulfur) is better in forming H-bonding at the binding sites, of which results in improved activity, particularly antimicrobial activity. In the same respect, GK505.2Py, GK506.2Fn, GK506.2Im, GK517.2, GK517.3, GK517.4, GK524, and GK585 also differ from each other. For example, although GK506.2Im and GK524 each contain an aromatic 5-membered ring at the tether, both compounds have a different physical appearance, solubility, biological activity, and toxicity due to the differences between the tethered rings.

Also contemplated herein are use of dimers previously shown to have anti-cancer activity for antimicrobial activity. Such compounds are characterized as either having two fused tricyclic (e.g., β-carbolines such as harmaline) or two fused bicyclic (e.g., quinoline and isoquinoline) moieties with a central linker, and having antimicrobial activity. Exemplary compounds are described in detail in U.S. Pat. No. 10,947,253, filed Aug. 5, 2019 and issued Mar. 16, 2021, incorporated by reference herein. For example, such compounds comprise two fused tricyclic moieties with a linker, and have the structure:

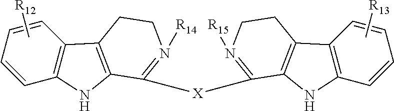

where R12 and R13 are independently selected from the group consisting of H, C1-C4 alkyl groups, and C1-C4 alkoxy groups, R14 and R15 are independently selected from the group consisting of nothing, H, and C1-C2 alkyl groups, and X is selected from the group consisting of (CH2)3 and C3-C8 geminal alkyl groups having a carbon atom therein with two functional groups bound to the carbon atom; the functional groups are independently selected from the group consisting of C1-C3 alkyl groups, C1-C3 alcohols, and metal atoms. In certain embodiments, R12 and R13 are each methoxy groups, R3 and R4 are nothing, and X is either the propyl moiety (CH2)3 or a C3 geminal alkyl groups, where the functional groups are both methyl groups. As used herein, where any R substituent bond line extends into an indeterminant position of a ring, the R substituent can be bound to any possible ring position. A preferred such compound is:

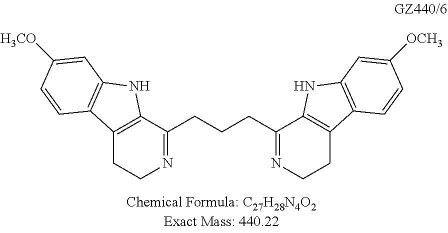 [SMILES: COc1ccc2c3c([nH]c2c1)C(CCCC1=NCCc2c1[nH]c1cc(OC)ccc21)=NCC3]

Another class of such compounds comprises two fused bicyclic moieties with an alkyl phenyl linker according to the following structures:

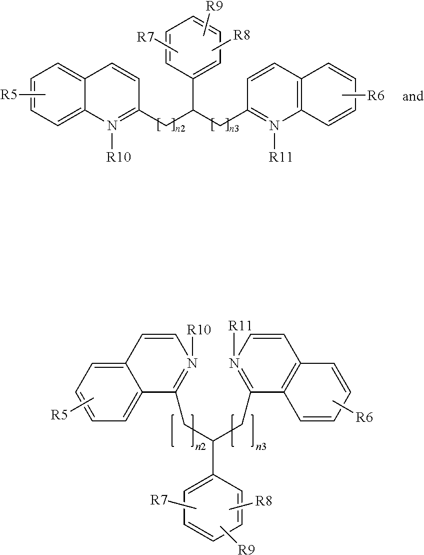

where R5 and R6 are independently selected from the group consisting of H, C1-C4 alkyl groups, and C1-C4 alkoxy groups, R7, R8, and R9 are independently selected from the group consisting of H, OH, C1-C4 alkoxy groups, C1-C4 alkyl groups, —N2, and CH2N2, and n2 and n3 are independently 1-4, and R10 and R11 are independently selected from the group consisting of nothing, H, and C1-C2 alkyl groups.

Certain representative compounds in accordance with this class include:

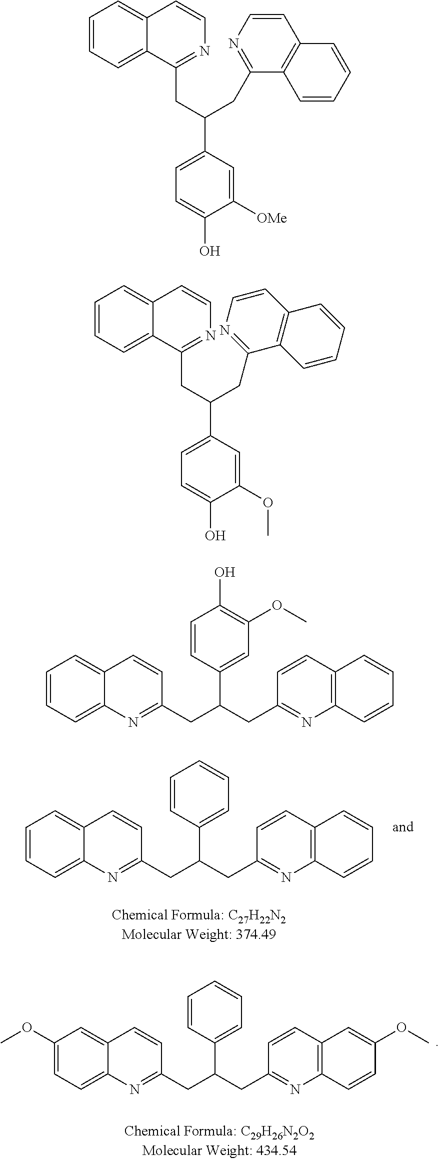

The present disclosure also contemplates variations on any of the foregoing structures, including “variants thereof” which is defined as the isomers, tautomers, enantiomers, esters, derivatives, metal complexes (e.g., Cu, Fe, Zn, Pt, V), prodrugs, solvates, or metabolites, and pharmaceutically acceptable salts thereof. “Isomers” refers to each of two or more compounds with the same formula but with at different arrangement of atoms, and includes structural isomers and stereoisomers (e.g., geometric isomers and enantiomers); “tautomers” refers to two or more isometric compounds that exist in equilibrium, such as keto-enol and imine and enamine tautomers; “derivatives” refers to compounds that can be imagined to arise or actually be synthesized from a defined parent compound by replacement of one atom with another atom or a group of atoms; “solvates” refers to interaction with a defined compound with a solvent to form a stabilized solute species; “metabolites” refers to a defined compound which has been metabolized in vivo by digestion or other bodily chemical processes; “prodrugs” refers to defined compound which has been generated by a metabolic process; and “pharmaceutically acceptable salts” with reference to the components means salts of the components which are pharmaceutically acceptable, i.e., salts which are useful in preparing pharmaceutical compositions that are generally safe, non-toxic, and neither biologically nor otherwise undesirable and are acceptable for human pharmaceutical use, and which possess the desired degree of pharmacological activity. Such pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use, P. H. Stahl & C. G. Wermuth eds., ISBN 978-3-90639-058-1 (2008).

### Compositions

Antimicrobial compositions comprising (consisting essentially or even consisting of) above-described fused tricyclic compounds, particularly the above-described fused tricyclic β-carboline adducts and/or dimer compounds are also contemplated. The compositions may include additional pharmaceutically-acceptable ingredients and/or vehicles as a base carrier composition in which the active ingredients are dispersed. As used herein, the term “pharmaceutically-acceptable” means not biologically or otherwise undesirable, in that it can be administered to a subject without excessive toxicity, irritation, or allergic response, and does not cause any undesirable biological effects or interact in a deleterious manner with any of the other components of the composition in which it is contained. The terms “vehicle” or “carrier,” as used herein, mean one or more compatible base compositions with which the active ingredient (e.g., above-described compounds) is combined to facilitate the administration of ingredient, and which is suitable for administration to a patient. Such preparations may also routinely contain salts, buffering agents, preservatives, and optionally other therapeutic ingredients or adjuvants. The carrier would naturally be selected to minimize any degradation of the active ingredient and to minimize any adverse side effects in the subject, as would be well known to one of ordinary skill in the art. Pharmaceutically-acceptable ingredients include those acceptable for veterinary use as well as human pharmaceutical use. The term “adjuvant” is used herein to refer to substances that have immunopotentiating effects and are added to or co-formulated in a therapeutic composition in order to enhance, elicit, and/or modulate the innate, humoral, and/or cell-mediated immune response against the active ingredients. Use of the compounds in the manufacture of a medicament for treating microbial infections is also within the ambit of the invention.

### Synthesis Methods

### 1. Tricyclic Compounds

To synthesize the above-described fused tricyclic β-carboline adduct compounds, a first amount of harmaline is transferred to a flask, preferably a round bottom flask, with a solvent, preferably ethanol, methanol. In general, the weight ratio of harmaline to solvent is about 0.001:1 to about 1:1. In embodiments where ethanol is used, the weight ratio of harmaline to ethanol is about 0.001:1 to about 1:1. The flask is equipped with a condenser, and the solution is stirred for about 5 minutes to about 20 minutes, preferably about 10 minutes to about 15 minutes, to dissolve the harmaline. Then, a desired compound comprising an aldehyde moiety (i.e., desired aldehyde) is transferred to the flask. Advantageously, the desired aldehyde is selected based on the desired tricyclic β-carboline adduct. Next, the flask is setup for reflux at a normal/atmospheric pressure and a temperature of about 60° C. to about 120° C., preferably about 80° C. to about 100° C., for about 30 mins to about 72 hours, preferably about 24 hours to about 60 hours, and more preferably about 48 hours. In another embodiment, the flask is setup for reflux a temperature of about 60° C., about 65° C., about 70° C., about 75° C., about 80° C., about 85° C., about 90° C., about 95° C., about 100° C., about 105° C., about 110° C., about 115° C. for about 12 hours, about 18 hours, about 24 hours, about 30 hours, about 36 hours, about 42 hours, about 48 hours, about 54 hours, about 60 hours, about 66 hours, about 72 hours. One of ordinary skill in the art would understand that the reflux temperature and reflux time may need to be adjusted according to the properties of the desired aldehyde selected. In general, the weight ratio of harmaline to the desired aldehyde is 0.001:1 to about 1:0.001. After reflux, the solution is cooled to room temperature and then transferred to a freezer at about −70° C. to about 14° C., preferably about −4° C. for about 5 minutes to about 45 minutes, preferably about 15 minutes to about 30 minutes. In another embodiment, after reflux, the solution is cooled to room temperature and then transferred to a freezer at about −70° C. to about 14° C., preferably about −4° C. for about 12 hours to about 48 hours, preferably about 24 hours to about 36 hours. In one or more embodiments, the resulting tricyclic β-carboline adduct compound is then collected from the cooled reaction solution using vacuum filtration and washed with cold solvent, preferably ethanol. Advantageously, the resulting tricyclic β-carboline adduct compound has improved activity (e.g., antimicrobial activity) and comparatively less toxicity as compared to β-carboline monomers, tryptamines, and tryptamides.

### 2. Derivatives or Dimers

In other embodiments, the tricyclic β-carboline adduct compound may be synthesized into the above-described tricyclic β-carboline dimer compounds. Particularly, instead of cooling the above-described solution (comprising the tricyclic β-carboline adduct compound and the solvent) to room temperature (and transferring to a freezer), a second amount of harmaline is transferred to the flask. In general, the weight ratio of harmaline to solvent is about 0.001:1 to about 1:1, and the weight ratio of harmaline to adduct is about 0.1 to about 2. The solution is stirred for about 5 minutes to about 20 minutes, preferably about 10 minutes to about 15 minutes, to dissolve the harmaline. Then, a desired aldehyde is transferred to the flask. Preferably, the desired aldehyde is the same aldehyde used to synthesize the tricyclic β-carboline adduct. Next, reflux is continued at a temperature of about 60° C. to about 120° C., preferably about 80° C. to about 100° C., for about 30 mins to about 72 hours, preferably about 24 hours to about 60 hours, and more preferably about 48 hours at normal ambient pressure. In another embodiment, the flask is setup for reflux at a temperature of about 60° C., about 65° C., about 70° C., about 75° C., about 80° C., about 85° C., about 90° C., about 95° C., about 100° C., about 105° C., about 110° C., about 115° C. for about 12 hours, about 18 hours, about 24 hours, about 30 hours, about 36 hours, about 42 hours, about 48 hours, about 54 hours, about 60 hours, about 66 hours, about 72 hours at normal ambient pressure. One of ordinary skill in the art would understand that the reflux temperature and reflux time may need to be adjusted according to the properties of the aldehyde selected. For example, reactions using low flash point aldehydes need to be heated to low temperatures for couple of hours and then raised to reflux temperatures to avoid ignition of the reaction solution. In general, the weight ratio of harmaline to the desired aldehyde is 0.001:1 to about 1:0.001. After reflux, the solution is cooled to room temperature and then transferred to a freezer at about −70° C. to about 14° C., preferably about −4° C. for about 5 minutes to about 45 minutes, preferably about 15 minutes to about 30 minutes. In another embodiment, after reflux, the solution is cooled to room temperature and then transferred to a freezer at about −70° C. to about 14° C., preferably about −4° C. for about 12 hours to about 48 hours, preferably about 24 hours to about 36 hours. In one or more embodiments, the resulting tricyclic β-carboline dimer compound is collected from the cooled reaction solution using vacuum filtration and washed with cold solvent, preferably ethanol. Advantageously, the resulting tricyclic β-carboline dimer compound has improved activity (e.g., antimicrobial activity) and comparatively less toxicity as compared to β-carboline monomers, tryptamines, and tryptamides.

In embodiments where the resulting tricyclic β-carboline dimer compound comprises two harmaline moieties, about 0.1 to about 10 of a reducing agent (e.g., NaBH₄) may be added to about 0.1 to about 1 of the dimer compound comprising two harmaline moieties to form a tricyclic β-carboline dimer compound comprising two tetrahydro harmine moieties. In other embodiments, the dimer compound comprising two harmaline moieties may reacted with an oxidizing agent (e.g., 2,3-dichloro-5,6-dicyano-1,4-benzoquinone (DDQ)) in a compatible solvent (e.g., THF) and in the presence of an diacid catalyst (e.g., succinic acid) to form a tricyclic β-carboline dimer compound comprising two harmine moieties, as described in detail in US 2022/0064156, filed Sep. 1, 2020, which is incorporated, in its entirety, by reference herein.

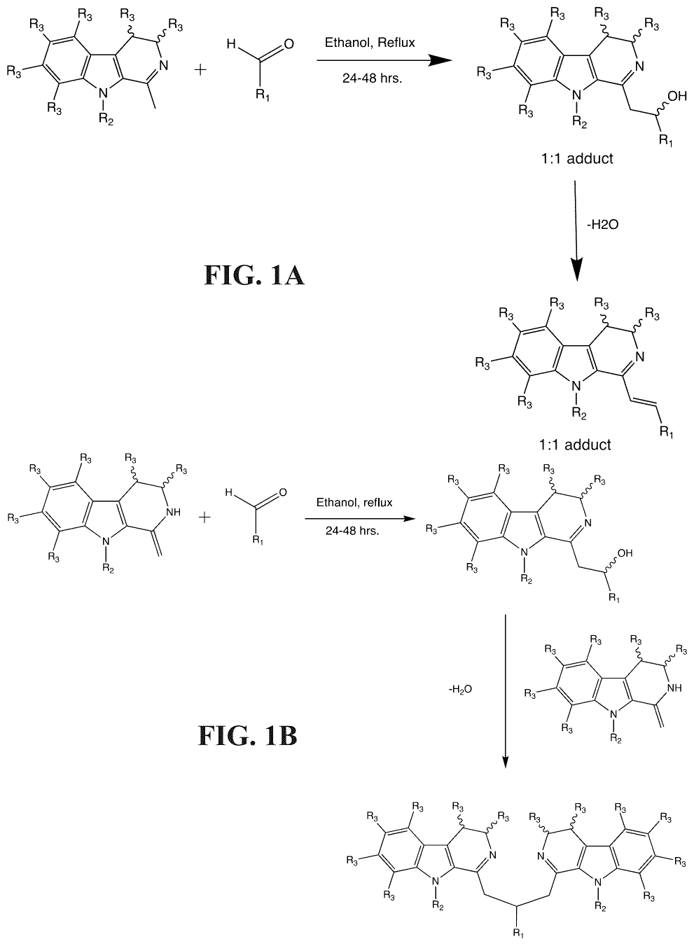

In most preferred embodiments, the synthesis method proceeds as shown in the reaction scheme in FIG. 1.

### 3. Equipment

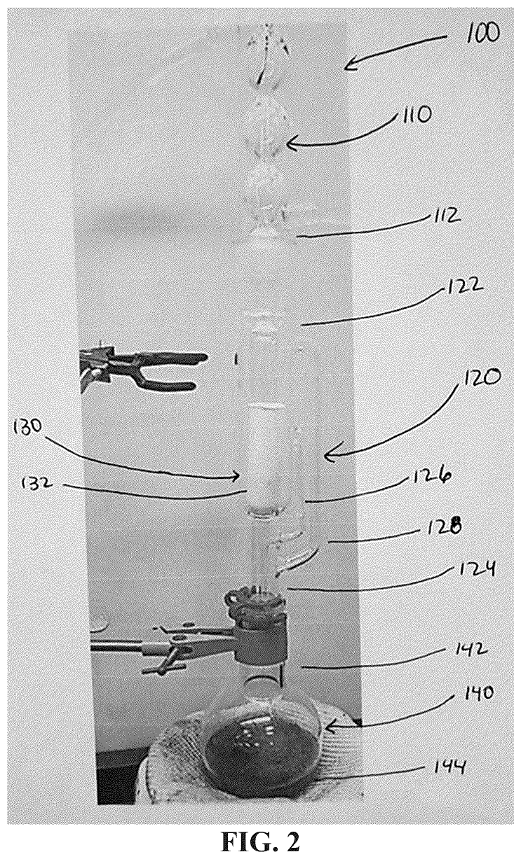

In preferred embodiments, instead of using a condenser, an extraction glassware set up is used to further facilitate the synthesis method described above. The use and advantages of the extraction glassware set up may be more readily understood with reference to the figures. FIG. 2 is an image of an extraction glassware set up 100 according to one aspect of the invention. Turning to FIG. 2, the extraction glassware set up 100 comprises a condenser 110, an extractor body 120, and a round bottom flask 140. Any suitable condenser 110 may be used in the set up 100, but, in one embodiment, an allihn condenser is used, which consists of a glass tube with a water jacket, and feature a series of bulbs that increase the surface area available for vapor condensation inside the tube. As shown in FIG. 2, a bottom opening of the condenser 112 is joined to a top opening of the extractor body 122. To avoid obstructing and/or slowing the reaction, it may be desired to restrict the amount of air entering the set up 100 through the joined portions of the condenser 110 and extractor body 120. Advantageously, in one embodiment, the bottom opening of the condenser 112 interlocks with the top opening of the extractor body 122 so as to prevent air from traveling into the extraction glassware set up 100. That is, preferably, the bottom opening of the condenser 112 is specifically designed to receive the top opening of the extractor body 122.

As illustrated in FIG. 2, the extractor body 120 has an inner external tube (i.e., the tube positioned closest to the extractor body) 126 and an outer external tube 128. In most embodiments, the extractor body 120 comprises an extraction thimble 130, which may be a quartz microfiber thimble, a glass microfiber thimble, or a cellulose thimble, preferably a cellulose thimble. The extraction thimble 130 further comprises molecular sieves 132. In one embodiment, the molecular sieves 132 have pores at a size range from about 3 angstroms to about 13, about 3 angstroms to about 5 angstroms, and more preferably about 4 angstroms. Further, the molecular sieves 132 are preferably activated (i.e., dried out in dehydrated form) to further improve the water absorption capability of the sieves 132. Furthermore, the extraction thimble 130 is porous so as to allow a solvent 144 to exit the thimble 130 but not so porous that the molecular sieves 132 can also exit the thimble 130. Finally, the round bottom flask 140, particularly the top opening of the flask 142, is attached to a bottom opening of the extractor body 124. Though a round bottom flask is pictured, any suitable flask may be used. The extraction glassware set up 100 may be scaled-up or down as desired, and the process of doing so is within the knowledge of one having ordinary skill in the art.

Advantageously, the extraction glassware set up 100 simultaneously removes water from the solvent 144 without losing the solvent 144. As the round bottom flask 140 containing the solvent 144 is heated, the hot solvent vapors rise in the outer external tube of the extraction body 128, enter the water-cooled condenser 120, and condense. The condensed solvent 144 drips into the extraction thimble 130 and collects in the thimble 130 until the solvent level 144 reaches the top of the inner external tube of the extractor body 126. Once the solvent reaches this level, it exits the thimble 130, traveling through the molecular sieves 132 to do so. By traveling through the molecular sieves 132, any water molecules present with the solvent 144 are substantially removed. Then, the extracted solvent 144 returns to the round bottom flask 140. This process continues for a desired duration, and by the end of this process, the solvent 144 loses any water present to the activated molecular sieves.

### 4. Additional Polycyclic Compounds

Variations on the reaction schemes can be carried out using alternative solvent systems to yield additional polycyclic compounds. In this reaction, DI water was added to the reaction flask. This method was established to specifically synthesize Harmaline and Aldehyde 1:1 adducts. No Harmaline dimer was detected when DI water used in the reaction flask. However, the amine of the aldehyde reacted with second aldehyde (see mechanism) to form this new polycyclic structure.

### Treatment Methods

The above-described fused tricyclic compounds, dimers thereof, variants thereof, and pharmaceutically acceptable salts thereof, and/or antimicrobial compositions comprising these compounds are useful for inhibiting microbial activity and/or a microbial infection in vitro, ex vivo, or in vivo. The microorganisms may be contacted with compounds and/or compositions in an effective amount in order to inhibit the growth, replication, and/or viability of the microorganism.

In one or more embodiments, the compounds and/or compositions may be administered to a subject (e.g., an animal) in need thereof. The subject may be suffering from, suspected of having, or at risk of exposure to or developing a microbial infection. In one or more embodiments, a therapeutically effective amount of the compounds and/or compositions is administered to the subject in order to inhibit microbial activity in the subject. Inhibition of microbial activity will likewise lessen the severity of the infection and/or its attendant symptoms and/or the duration of the infection. Such usages would typically be in vivo.

Advantageously, the above-described fused tricyclic compounds, particularly the above-described fused tricyclic β-carboline adducts and/or dimer compounds, do not show genotoxicity (when tested without S9 activation) at concentrations below 85 μM, and some tricyclic β-carboline dimer compounds do not show genotoxicity at concentrations below 90 μM, below 95 μM, and below 100 μM. As used herein, the term “S9 activation” refers to crude liver enzyme extract. When tested with S9 activation, some fused tricyclic compounds do not show genotoxicity at concentrations below 50 μM, below 65 μM, and below 70 μM. As used herein, the term “genotoxicity” refers to the capability of a substance to damage the genetic information of cells, and is measured at a UMU induction ratio value of less than 1.5.

Though the above-described fused tricyclic compounds (and/or compositions thereof) possess anti-protozoa and/or anti-fungal properties, the compounds are preferably used to inhibit bacteria and/or viruses.

In one or more embodiments, the above-described fused tricyclic compounds, preferably the tricyclic β-carboline adducts and/or dimer compounds, (and/or compositions thereof) are effective for inhibiting bacterial or viral activity. As used herein, bacterial or viral “activity” refers to the activity, growth, replication, and/or viability of a virus or bacteria. The bacteria may be gram positive bacteria (e.g., S. aureus, E. faecalis) and/or drug resistant bacterial strains (e.g., S. aureus (methicillin and oxacillin resistant), E. faecalis (vancomycin resistant and sensitive to teicoplanin)) thereof, or the bacteria may be gram negative bacteria (e.g., E. coli, P. aeruginosa) and/or drug resistant bacterial strains thereof. Viruses include HIV, Covid-19, Hepatitis. As exemplified in the working examples, certain compounds of the invention are particularly effective as against one or the other of a gram negative or gram positive bacteria, whereas certain other compounds have broad spectrum efficacy against both types of bacteria.

In one or more embodiments, the methods comprise (consist essentially or even consist of) contacting a bacterium or virus with an effective amount of the above-described compounds and/or compositions to inhibit bacterial or viral replication. In another embodiment, the methods comprise (consist essentially or even consist of) administering a therapeutically or prophylactically effective amount of the above-described compounds and/or compositions to a subject in need thereof. The subject may be at risk of bacterial or viral infection or exposure to bacteria or a virus. Such individual may be symptomatic or asymptomatic. Thus, “therapeutic” use of the compounds and/or compositions refers to processes that are intended to produce a beneficial change in an existing condition (e.g., bacterial or viral infection) of a subject, such as by reducing the severity of the clinical symptoms and/or effects of the infection, and/or reducing the duration of the infection/symptoms/effects. Likewise, “prophylactic” use refers to processes that are intended to inhibit or ameliorate the effects of a future bacterial or viral infection to which a subject may be exposed (but is not currently infected with). In some cases, the compounds and/or compositions may prevent the development of observable morbidity from bacterial or viral infection (i.e., near 100% prevention). In other cases, the compounds and/or compositions may only partially prevent and/or lessen the extent of morbidity due to the bacterial or viral infection (i.e., reduce the severity of the symptoms and/or effects of the infection, and/or reduce the duration of the infection/symptoms/effects). In either case, the compounds and/or compositions are still considered to “prevent” the target infection.

More particularly, the invention relates to methods comprising (or consisting essentially of or even consisting of) the step of administering to a subject suffering from a bacterial or viral infection the above-described fused tricyclic compounds, dimers thereof, and/or compositions, as well as variants thereof, and pharmaceutically acceptable salts thereof. For example, in the context of the present invention, inhibition of bacterial or viral replication refers to a decrease in amount or speed of bacterial or viral replication as compared to a baseline or control.

In some embodiments there is a viral load reduction of at least about 10%, about 15%, about 20%, about 30%, about 40%, about 50%, about 60%, about 70%, about 75%, about 80%, about 85%, about 90%, about 95%, about 99%, and about 100%.

In one or more embodiments, the above-described fused tricyclic compounds, particularly the tricyclic β-carboline dimer compounds, have a minimum inhibitory concentration (MIC) of about 6.5 μg/mL to about 1500 μg/mL, preferably about 85 μg/mL to about 1500 μg/mL against gram negative bacteria.

In one or more embodiments, the above-described fused tricyclic compounds, particularly the tricyclic β-carboline dimer compounds, also have a MIC of about 3 μg/mL to about 90 μg/mL, preferably about 20 μg/mL to about 85 μg/mL, more preferably about 25 μg/mL to about 80 g/mL, and most preferably about 30 μg/mL to about 75 μg/mL against gram positive bacteria, and have a minimum bactericidal concentration (MBC) (i.e., killed 99.9% of bacteria) of about 50 g/mL to about 150 μg/mL, preferably about 55 μg/mL to about 130 μg/mL, more preferably about 60 μg/mL to about 115 μg/mL, and most preferably about 65 μg/mL to about 105 μg/mL against gram positive bacteria.

In one or more embodiments, the above-described fused tricyclic compounds, particularly the tricyclic β-carboline dimer compounds, have a MIC of about 0.5 μg/mL to about 75 μg/mL, preferably about 1 μg/mL to about 50 μg/mL, more preferably about 2.5 μg/mL to about 40 μg/mL, and most preferably about 5 μg/mL to about 30 μg/mL against drug resistant gram positive bacteria strains up to about 12 to about 36 hours, preferably up to about 24 hours.

Furthermore, the above-described fused tricyclic compounds, particularly the tricyclic β-carboline dimer compounds, have a MIC of 1 μg/mL about to about 130 μg/mL, preferably about 5 μg/mL to about 100 μg/mL, more preferably about 10 μg/mL to about 70 μg/mL, and most preferably about 15 μg/mL to about 65 μg/mL against drug resistant gram positive bacteria strains up to about 36 hours to about 102 hours, preferably up to about 48 hours to about 90 hours.

Finally, in other embodiments, the above-described fused tricyclic compounds, particularly the tricyclic β-carboline dimer compounds, have an MBC of about 10 μg/mL to about 140 μg/mL, preferably about 20 μg/mL to about 110 μg/mL, more preferably about 25 μg/mL to about 85 g/mL, and most preferably about 30 μg/mL to about 65 μg/mL against drug resistant gram positive bacteria strains.

It will be appreciated that the efficacy of tricyclic β-carboline dimer compounds against gram negative bacteria (and drug resistant bacterial strains thereof) is about 10 to 20 times, preferably about 12 to 18 times, and more preferably about 15 times more than the efficacy of the tricyclic β-carboline monomers (e.g., harmaline) against gram negative bacteria (and drug resistant bacterial strains thereof). The efficacy of tricyclic β-carboline dimer compounds with a substitution/substituent at the tether (e.g., GK506.2Im, GK517.3, GK580 and GK524 compounds) against gram negative bacteria (and drug resistant bacterial strains thereof) is about 0.75 to 2.5 times, preferably about 1.25 to 2.25 times, and more preferably about 1.75 times more than the efficacy of tricyclic β-carboline dimer compounds without a substitution/substituent (e.g., GZ440/6) against gram negative bacteria (and drug resistant bacterial strains thereof).

Those of ordinary skill in the art can readily determine whether or not bacterial or viral replication or load has been inhibited and to what extent. Thus, an “effective amount” to inhibit bacterial or viral replication refers to the amount of the above-described fused tricyclic compounds that results in a reduced level of bacterial or viral replication and thus a reduced amount of bacterial or viral load. Correspondingly, such reductions in bacterial or viral load will advantageously lead to an amelioration, improvement, or decrease in one or more symptoms associated with bacterial or viral infection. Assays for bacterial or viral replication also provide one with the ability to determine the efficacy of bacterial or viral inhibitors and are well known to those skilled in the art. Such assays may be conducted in vivo or in vitro.

Exemplary methods of and compounds for inhibiting viral activity can also be found in U.S. application Ser. No. 17/108,851, filed Dec. 1, 2020, which is incorporated, in its entirety, by reference herein.

The fused tricyclic compounds (and/or composition) as administered should be of high purity, e.g., at least about 95% (more preferably at least about 99%) pure. Inasmuch as the compounds are synthesized, purity levels can be controlled. The compounds can be directly used in partial or essentially completely purified forms, or can be modified as indicated above. The compounds may be in crystalline or amorphous forms, and may be lyophilized.

The above-described fused tricyclic compounds may be administered or applied to a subject in any convenient manner, such as by oral, rectal, nasal, ophthalmic, parenteral (including intraperitoneal, gastrointestinal, intrathecal, intravenous injection, cutaneous (e.g., dermal patch), subcutaneous (e.g., injection or implant), or intramuscular), and topical administrations. The dosage forms of the invention may be in the form of liquids, gels, suspensions, solutions, or solids (e.g., tablets, pills, or capsules). Moreover, therapeutically effective amounts of the compounds of the invention may be co-administered with other antimicrobial compound(s), where the two products are administered substantially simultaneously or in any sequential manner.

Levels of dosing to subjects of the above-described fused tricyclic compounds are quite variable owing to factors such as the patient's age, patient's physical condition, and the severity of the disease. In general, however, regardless of the dosage form or route of administration employed, the compounds (and/or compositions thereof) should be dosed of from about 5 mg per day to about 2,000 mg per day, and more usually from about 100 mg per day to about 800 mg per day. Such dosages may be based on a single administration per day, but more usually multiple administrations per day.

It will be appreciated that therapeutic and prophylactic methods described herein are applicable to humans as well as any suitable animal, including, without limitation, dogs, cats, and other pets or captive animals (e.g., zoo animals, research subjects), as well as, rodents, primates, horses, cattle, pigs, etc. The methods can be also applied for clinical research and/or study. Additional advantages of the various embodiments of the disclosure will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present disclosure encompasses a variety of combinations and/or integrations of the specific embodiments described and claimed herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the disclosure. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

This example describes the general reaction procedures for synthesizing various new fused tricyclic compounds.

### 1. General Method Using Novel Reaction Setup; Novel Fused Tricyclic Compounds Synthesized Using Novel Reaction Setup

The following exemplifies a new reaction setup developed to facilitate the synthesis of the fused tricyclic compounds. The general procedure is described in this Part, and this procedure is used to synthesize specific compounds in Example 3. To synthesize a desired harmaline dimer compound, harmaline is transferred to a round bottom flask with ethanol. In general, the weight ratio of harmaline to ethanol is about 0.001:1 to about 1:1. Harmaline and aldehyde were transferred to a round bottom flask with ethanol. The flask is equipped with a conventional condenser, flask is setup for reflux for 20-48 hours under nitrogen. In general, the weight ratio of harmaline to aldehyde is 0.001:1 to about 1:0.001.

In the new set up, the conventional condenser above is replaced with an extraction glassware set up comprising a condenser, extractor body, and a round bottom flask. A cellulose extraction thimble with activated molecular sieves (4 Å, 325 mesh particle size) is placed in the extractor body (see FIG. 2). As the round bottom flask containing ethanol is heated, hot ethanol vapors rise in the outer tube of the extraction body, enter the water-cooled condenser, and condense. The condensed ethanol drips into the porous cellulose thimble. The condensed ethanol collects in the thimble until the ethanol level reaches the top of the inner bent tube/siphon. Once the ethanol reaches this level, it exits the thimble, traveling through the activated molecular sieves to do so. By traveling through the molecular sieves, the water present with the ethanol is substantially removed. Then, the extracted ethanol returns to the round bottom flask. This process continues for 48 hours, and during this time, ethanol loses any water present to the activated molecular sieves. The desired harmaline dimer compound is precipitated in the solution towards the end of the reaction time. The precipitation formation is visible, and the reaction can be stopped when no more precipitation is observed. After the reaction time, the solution is cooled to room temperature, and the flask is stored in a freezer overnight to improve the crystallization. Vacuum filtration is performed to collect the product, which is washed with cold ethanol. Air-dried compounds are stored in a freezer. The filtrate was concentrated on rotavapor.

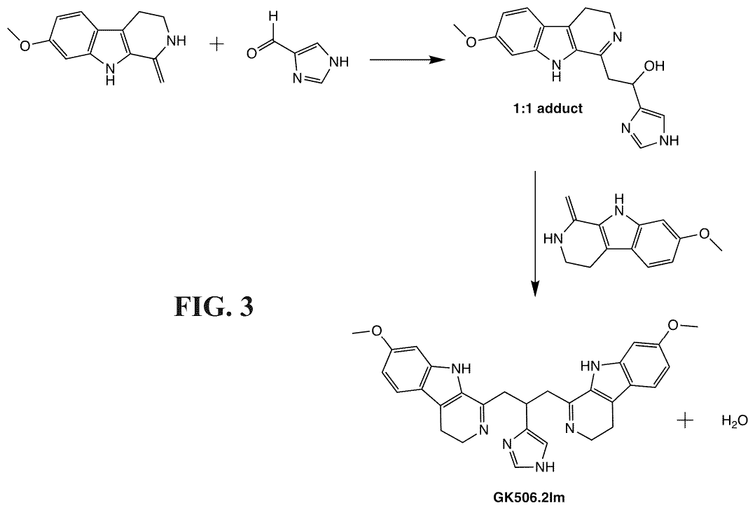

Notably, removing the water from ethanol helps to move the reaction forward. Please see in FIG. 3, the reaction mechanism of GK506.2Im to understand the importance of removing water that forms during the reaction. As shown in the reaction scheme in FIG. 3, one molecule of harmaline and aldehyde forms a 1:1 adduct, which reacts with a second harmaline molecule to form a harmaline dimer. The presence of water in the solution slows down the conversion of the adduct into harmaline dimer. Le Chatelier's principle can be applied to move the reaction in forward manner. The modification involves the use of extraction glassware with cellulose thimble filled with activated molecular sieves (see FIG. 2 and Example 1). Notably, the process of drying ethanol will not happen in case of a simple condenser attached to reaction flask. The new setup helps to furnish desired compound with high yield and purity, which cannot be achieved in typical Harmaline+Aldehyde reactions (see, e.g., Example 2).

The following novel harmaline dimer compounds were synthesized using the above-described reaction setup:

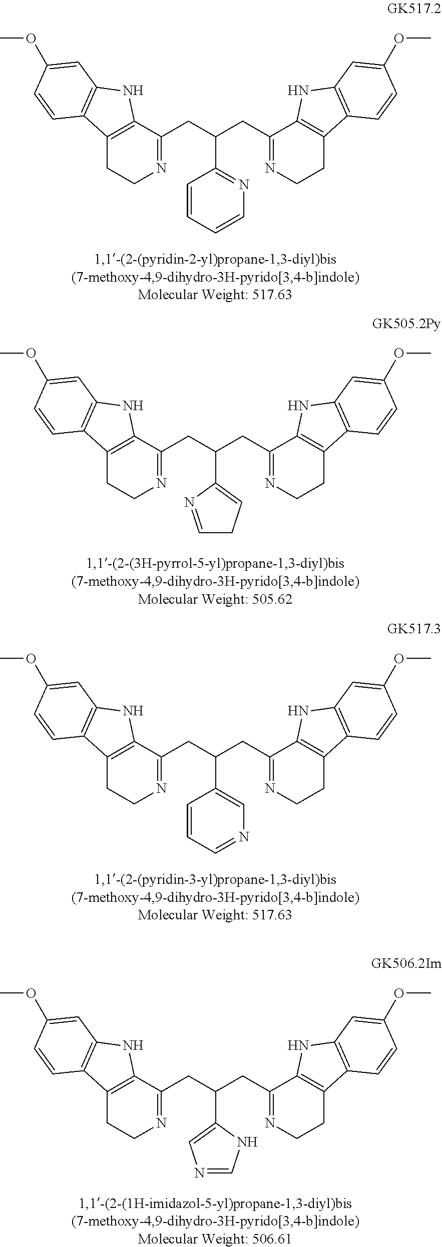

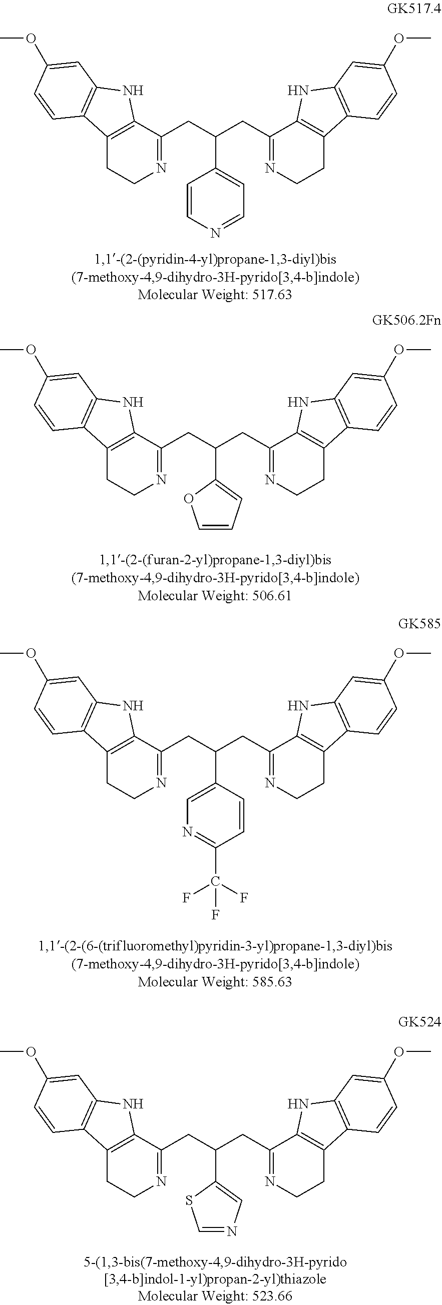

Samples were prepared for LC-MS analysis, and the data confirms that the solids collected were pure harmaline dimer molecules. Unreacted Harmaline and Harmaline aldehyde 1:1 adducts were detected in the filtrate. The use of molecular sieves significantly improved the yield and furnished pure compounds.

Using these new methods, various structures can be synthesized by refluxing harmaline with various aldehyde molecules in solvents like ethanol.

2. Harmine and Tetrahydro Harmine Dimer Compounds Synthesized from Harmaline Dimer Compounds Using Reducing and Oxidizing Agents

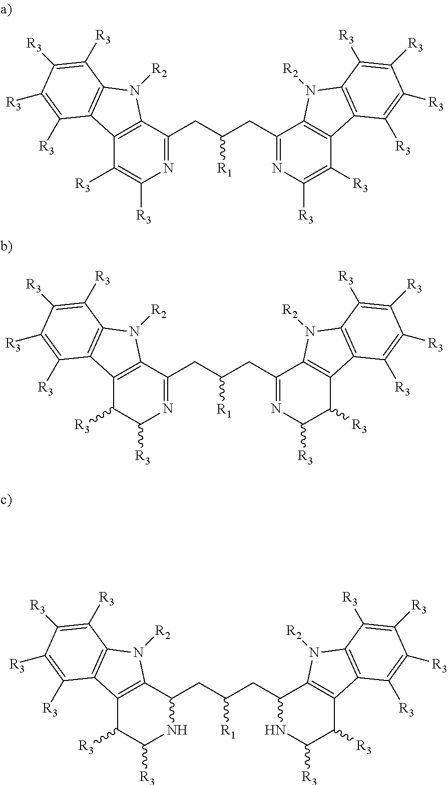

General representation of a) Harmine dimer b) Harmaline dimer c) Tetrahydro Harmine dimer structures.

Harmine dimers (a) above can be achieved by treating Harmaline dimers (b) with oxidizing agents like 2,3-dichloro-5,6-dicyano-1,4-benzoquinone (DDQ), as described in detail in US 2022/0064156, filed Sep. 1, 2020, which is incorporated, in its entirety, by reference herein. Briefly, the method comprises reacting harmaline with DDQ in the presence of a diacid catalyst. In an embodiment, the synthesis is carried out using a reaction mixture of harmaline, DDQ, THE solvent, and succinic acid; the reaction is carried out under an inert atmosphere with refluxing for a period of 4-12 hours. Similarly, Tetrahydro Harmine dimers (c) can be achieved by treating Harmaline dimers (b) with reducing agents like NABH₄ (as described in U.S. Pat. No. 10,947,253, issued Mar. 16, 2021, which is incorporated, in its entirety, by reference herein). See Scheme 2 in FIG. 4 for details.

### Example 2

### Synthesis of GK506.2Im

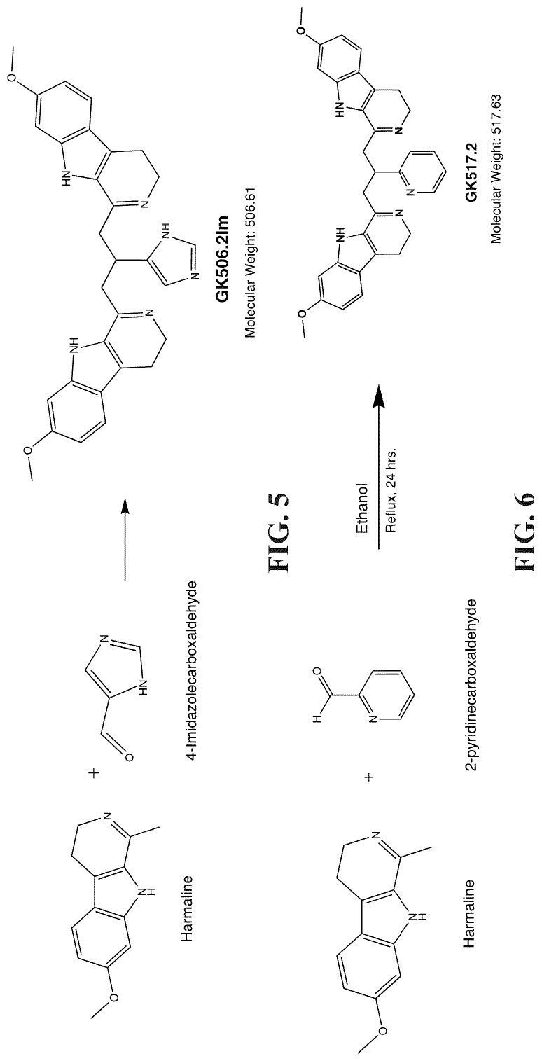

This example describes a specific synthesis protocol and resulting compound (GK506.2Im). As shown in the reaction scheme in FIG. 5, 3.34 grams of harmaline were transferred to a clean 250 mL round bottom flask containing 100 mL of ethanol followed by 0.5 grams of 4-imidazolecarboxaldehyde. The round bottom flask was attached with a condenser and set up for reflux for 24 hours under nitrogen at 100° C. The resulting solution was pale yellow in color and changed from wine red to dark brown with the heating. After 3 hours of reflux, formation of yellow solid crystals was observed. After 5 hours of reflux, the solution turned into a yellow suspension. The reflux was continued for 24 hours at a temperature of 100° C. After 24 hours of reflux, the solution was cooled to room temperature and then transferred to a freezer for 15-30 minutes for further crystallization. The resulting solid was collected using vacuum filtration and washed with cold ethanol to remove impurities. Recrystallisation was performed with ethanol to yield 1.36 grams (52%) of GK506.2Im (molecular formula: C₃₀H₃₀N₆O₂; IUPAC name: 1,1′-(2-(1H-imidazol-4-yl)propane-1,3-diyl)bis(7-methoxy-4,9-dihydro-3Hpyrido[3,4-b]indole) as a pure, light yellow, fluffy solid.

### Example 3

### Synthesis of GK517.2, GK517.3, GK517.4, GK524, GK585, and GZ440/6 Using Novel Extraction Glassware Set-up

GK517.2, GK517.3, GK517.4, GK524, GK585, and GZ440/6 were synthesized using the novel reaction setup method described in Example 1.

### 1. Synthesis of GK517.2

Referring generally to the reaction scheme in FIG. 6, 6 grams of harmaline were added to a 250 mL round bottom flask containing 100 mL of ethanol followed by 1 mL of 2-pyridinecarboxaldehyde, and the flask was set up for reflux at 100° C. Notably, instead of using a condenser, a unique extraction glassware set up was used to dry the solvent during the reaction (see FIG. 2 and Example 1). Molecular sieves (4 Å, 325 mesh particle size) were placed in the tumbler; and as a result, when liquid collected in the extraction glassware, the moisture was absorbed by the molecular sieves, and the ethanol was sent back to the flask.

Within 30 mins of reflux, the solution turned yellow, and precipitate formation was observed. The solution was refluxed for 20 hours and then was cooled to room temperature. Then, the round bottom flask was stored in a freezer for 15-30 minutes for further crystallization. The resulting solid was collected using vacuum filtration and washed with cold ethanol to yield 6.5 grams (60%) of GK517.2 (molecular formula: C₃₂H₃₁N₅O₂; IUPAC name: see Example 1). Recrystallization was not required as a pure compound was furnished from the reaction.

### 2. Synthesis of GK517.3

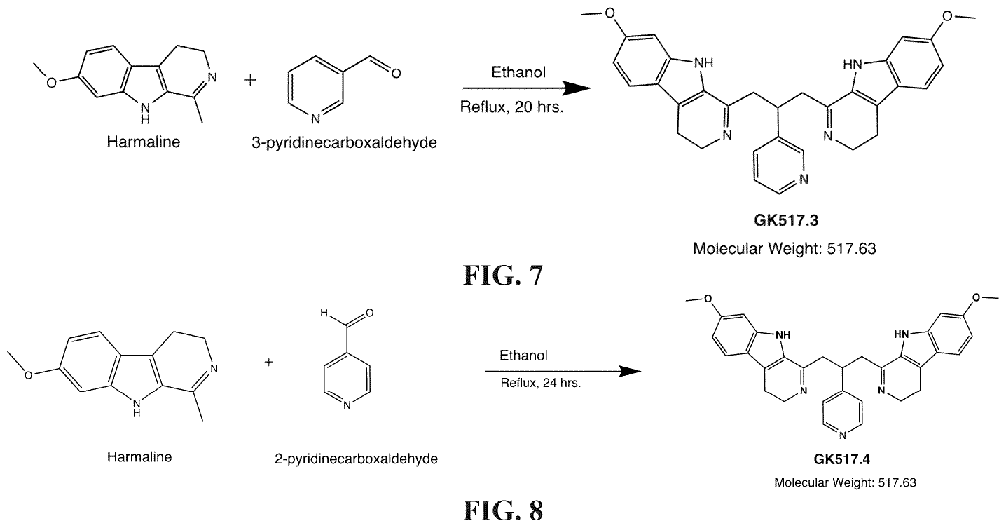

GK517.3 was prepared according to the synthesis method described in Part 1 of this Example, except 3-pyridinecarboxaldehyde was used instead of 2-pyridinecarboxaldehyde, as shown in FIG. 7. The resulting solid was collected using vacuum filtration and washed with cold ethanol to yield 6.7 grams (62%) of GK517.3 (molecular formula: C₃₂H₃₁N₅O₂; IUPAC name: 1,1′-(2-(pyridin-3-yl)propane-1,3-diyl)bis(7-methoxy-4,9-dihydro-3H-pyrido[3,4-b]indole)) as a light yellow colored solid. Recrystallization was not required as a pure compound was furnished from the reaction.

### 3. Synthesis of GK517.4

GK517.4 was prepared according to the synthesis method described in Part 1 of this Example, as shown in FIG. 8. The resulting solid was collected using vacuum filtration and washed with cold ethanol to yield 6.53 grams (60.42%) of GK517.3 (molecular formula: C₃₂H₃₁N₅O₂; IUPAC name: see Example 1). Recrystallization was not required as a pure compound was furnished from the reaction.

### 4. Synthesis of GK524

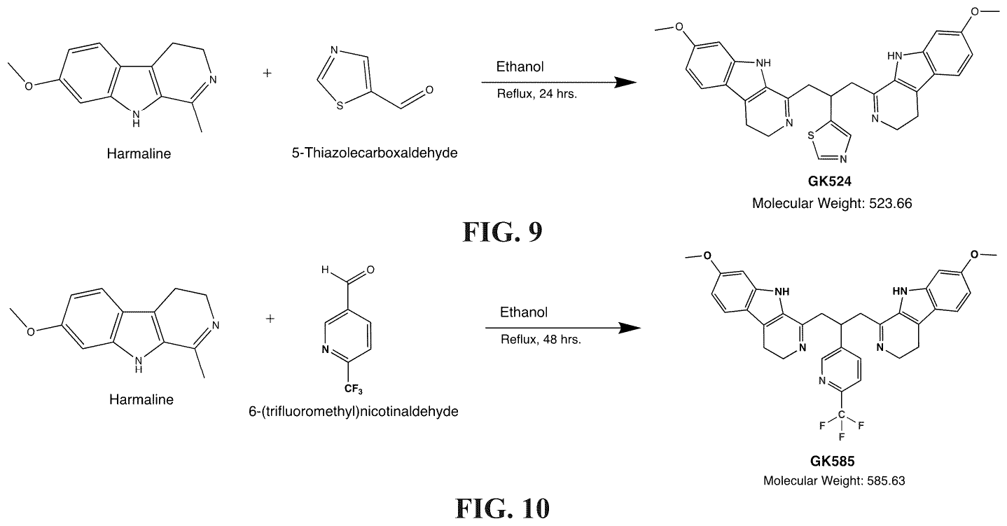

Referring generally to the reaction scheme in FIG. 9, 0.41 mL of 5-thiazolecarboxaldehyde, 1 eq (0.005 mole), was added to a round bottom flask followed by 2.87 grams of harmaline, 2.64 eq (0.013 moles). Then, 100 mL of 200 proof ethanol was added to the flask. Extraction glassware with molecular sieves (see Example 1) was matched to the flask and setup for reflux at 100° C. for 24 hours.

After 24 hours, the solution was cooled to room temperature and kept in a freezer overnight. The resulting solid was collected using vacuum filtration to yield 1.63 grams (61.24%) of GK524 (molecular formula: C₃₀H₂₉N₅O₂S; IUPAC name: see Example 1) as a pale-yellow solid.

### 5. Synthesis of GK585

Referring generally to the reaction scheme in FIG. 10, 1.61 grams of harmaline and 0.5 grams of 6-(Trifluoromethyl)pyridine-3-carboxaldehyde were added to a flask containing 100 mL of 200 proof ethanol. After ethanol was added, extraction glassware with molecular sieves (see Example 1) was matched to the flask and setup for reflux. The solution was heated to 80° C. for 2 hours. Then, the temperature of the solution was raised to 100° C., and reflux was continued for an additional 48 hours.

The solution was cooled to room temperature and stored in the freezer for 36 hours. The resulting solid was collected using vacuum filtration to yield 1.19 grams (71.16%) of GK585 (molecular formula: C₃₃H₃₀F₃N₅O₂; IUPAC name: see Example 1) as a pale yellow colored solid. Notably, increasing the reaction time to 48 hours and the freezer time to 36 hours improved the yield.

### 6. Synthesis of GZ440/6

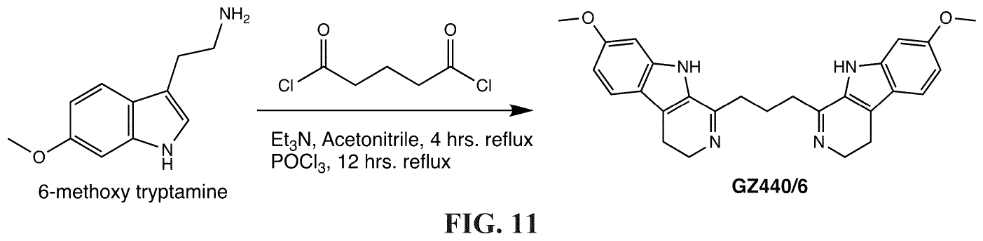

As generally shown in the reaction scheme in FIG. 11, 300 mg of 6-methoxy tryptamine, 2 eq (0.0016 moles), was added to a flame dried flask in glove bag under nitrogen. The flask was equipped with a condenser, and the setup was transferred to a hood. 100 mL of freshly distilled acetonitrile was added to the flask using a glass syringe. Then, 0.22 mL of Et₃N, 2 eq (0.0016 moles), was added to the flask followed by 133 μL of glutaryl chloride, 1 eq (0.0008 moles). The resultant solution was heated to reflux at 90-95° C. for 4 hours. After 4 hours, 1.47 mL of POCl₃, 20 eq (0.016 moles), was added to the flask slowly using a glass syringe. Notably, white dense fumes formed during the addition of POCl₃, and the formation of a greenish yellow colored precipitation was observed in 60 minutes of the reflux. Then, reflux was continued for 12 more hours. After the reaction was completed, the solution was cooled to room temperature. Further cooling was achieved by keeping the flask in an ice bath for 30 minutes. The resulting solid was collected using vacuum filtration and washed with cold ethanol to furnish 290 mg (83.5%) of yellow colored GZ440/6.

If impurities are detected, a solvent system including 5-10% methanol in dichloromethane can be used to perform column purification, and recrystallisation with ethanol can help in removing salts formed during the reaction. 2-5% isopropyl amine can be used to help prevent the reaction of the compound with the acidic nature of silica or dichloromethane during purification.

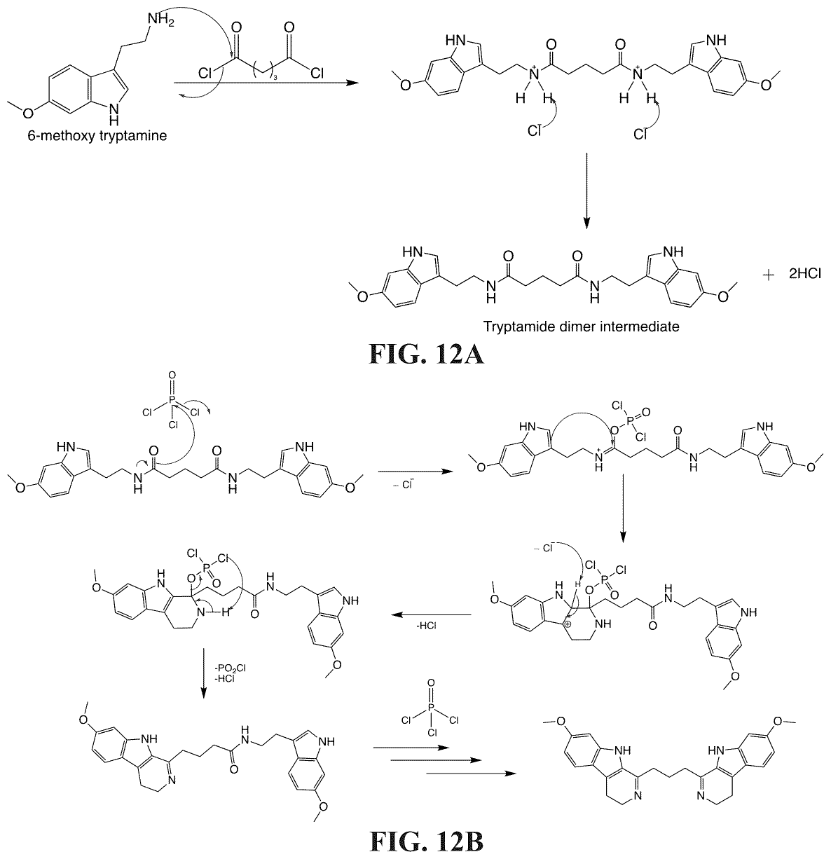

The reaction mechanism of the process described in Part 3 of this Example is shown in FIG. 12A. The scheme illustrates the reaction between tryptamine and a diacid chloride to furnish tryptamide dimers (i.e., an S_{N}2 reaction). 2 equivalent of tryptamine reacts with 1 equivalent of diacid chloride furnishes 1 equivalent of tryptamide dimer and 2 equivalents of HCl. The scheme in FIG. 12B illustrates the reaction between phosphoryl chloride and tryptamide dimer intermediate to furnish GZ440/6 (i.e., a Bischler-Napieralski reaction).

### Example 4

### Chemical Analysis of GK506.2Im, G GK506.2Fn, K517.2, GK517.3, GK517.4, GK524, GK585, and GZ440/6

Liquid chromatography-Mass spectrometry (LC-MS) was used to analyze GZ440/6, GK506.2Im, GK506.2Fn, GK517.2, GK517.3, GK517.4, GK524, and GK585. 1 mg of each compound was dissolved in 1 mL of LC-MS grade methanol. Then, 100 μL of the resulting solution was transferred to a vial containing 900 μL of LC-MS grade acetonitrile and water, forming a sample (for each compound) for analysis. To obtain a mass spectrum of the samples, a Xevo G2-Xs QT of system equipped with a Waters Acquity reversed-phase UPLC column (2.1*100 mm C18 1.7 uM) was used. A 75% water and 25% acetonitrile solvent system was used to analyze 1 μL of each sample with 8 minutes runtime.

LC-MS yielded the following results:

| Compound | Result |
|---|---|
| GK506.2Im | [M + H⁺] calculated for C₃₀H₃₀N₆O₂ 507.25; |
| | found 507.25 |
| GK506.2Fn | [M + H+] calculated for C31H30N4O3 507.24; |
| | found 507.23 |
| GK517.2 | [M + H⁺] calculated for C₃₂H₃₁N₅O₂ 518.26; |
| | found 518.25 |
| GK517.3 | [M + H⁺] calculated for C₃₂H₃₁N₅O₂ 518.26; |
| | found 518.25 |
| GK517.4 | [M + H⁺] calculated for C₃₂H₃₁N₅O₂ 518.26; |
| | found 518.25 |
| GK524 | [M + H⁺] calculated for C₃₀H₂₉N₅O₂S 524.21; |
| | found 524.21 |
| GK585 | [M + H⁺] calculated for C₃₃H₃₀F₃N₅O₂ 586.24; |
| | found 586.24 |
| GZ440/6 | [M + H⁺] calculated for C₂₇H₂₈N₄O₂ 441.23 |
| | was found 441.23 |

### Example 5

### Antibacterial Activity of Fused Tricyclic Compounds

### 1. Protocols

Synthesized compounds were tested for antibiotic activity towards the standard and drug resistant bacterial strains provided in Table 1 below. The concentration of compound required to visibly stop the growth of bacteria is known as minimum inhibitory concentration (MIC). Different concentrations of the compounds are tested for the exact same number of bacteria, and the growth is monitored with time. The most used method to determine MIC is broth microdilution assay which is followed by minimum bactericidal concentration (MBC) assay for determining the minimum concentration required to kill the bacteria.

### Single Compound MIC Determination Via Broth Microdilution Assay

Bacterial strains were purchased from the American Type Culture Collection (ATCC) for the studies. Table 1 shows the list of bacterial strains used for testing the antibacterial activities of the synthesized compounds.

TABLE 1
List of Bacterial strains used in MIC assay.

| Bacterial | | Gram stain | Medium | Incubation |
|---|---|---|---|---|
| Strain | ATCC ID | Test | (Agar/Broth) | (° C.) |
| E. coli | 25922 | Negative | Trypticase Soy | 37 |
| | | | (TSA/B) | |
| P. aeruginosa | 10145 | Negative | Nutrient (NA/B) | 37 |
| S. aureus subsp. | 29213 | Positive | Trypticase Soy | 37 |
| aureus Rosenbach | | | (TSA/B) | |
| MRSA: S. aureus | 43300 | Positive | Trypticase Soy | 37 |
| Resistant to | | | (TSA/B) | |
| methicillin and | | | | |
| oxacillin | | | | |
| E. faecalis | 29212 | Positive | Trypticase Soy | 37 |
| | | | with Defibrinated | |
| | | | Sheep Blood | |
| | | | (TSA/B + BSB) | |
| VREF: E. faecalis | 51299 | Positive | Trypticase Soy | 37 |
| Resistant to | | | with Defibrinated | |
| Vancomycin | | | Sheep Blood | |
| | | | (TSA/B + DSB) | |

The list involves two gram-negative bacteria strains of Escherichia co/i (E. coli; ATCC TD: 25922) and Pseudomonas aeruginosa (P. aeruginosa; ATCC TD: 10145) and two gram-positive bacteria, Staphylococcus aureus (S. aureus; ATCC TD: 29213) and Enterococcus faecalis (E. faecalis; ATCC TD: 29212) and their drug resistant strains. The MIC assay procedure follows the guidelines set by the Clinical Laboratory Sciences Institute (CLSI).

The MIC assay has the following steps:

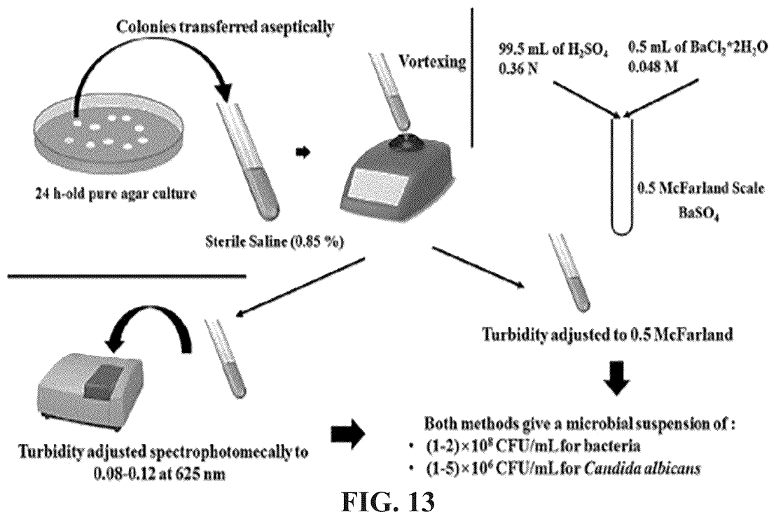

(a) Preparation of Inoculum: The first step is to prepare a bacterial culture containing special medium and bacteria as shown in the Scheme in FIG. 13.

To do this, bacteria were transferred from pure agar culture, aseptically transferred in 5 mL of Cation-Adjusted Mueller-Hinton Broth (CAMHB). The broth was incubated overnight at 35° C. to stationary phase to achieve a turbidity suspension. A spectrophotometer (SpectraMax ID5) was used to measure the turbidity (optical density—OD at 625 nm). The inoculum was prepared by adjusting the turbidity of overnight culture to 0.5 McFarland, which is equal to the defined number of bacteria 1×10⁸ CFU/ml.

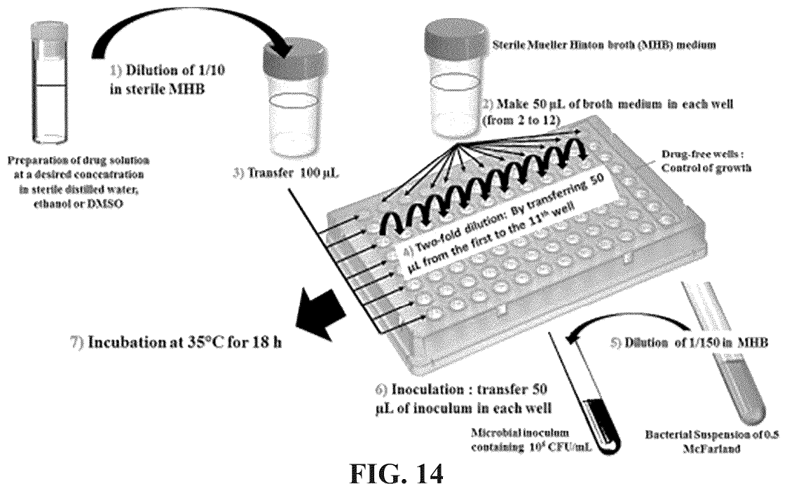

(b) Compound Dilution: All compounds used in the study were dissolved in sterile ultra-pure DMSO (VWR International, PA, USA) and maintained at −80° C. Stock solution of the compound was freshly prepared the day of use or is prepared from a frozen stock solution stored at −80° C. As illustrated in FIG. 14, the steps involved in broth microdilution assay for antibacterial testing according to CLSI protocol are shown in the scheme above. The compound solution was prepared at a desired concentration in DMSO, and 1/10 dilution was performed in a sterile CAMHB medium. This 10-fold dilution of stock solution was used for a 2-fold serial dilution in a 96-well plate to observe an inhibitory effect of the compound on bacterial growth. 100 μL of diluted drug solution was transferred to a first column of the 96-well plate. 50 μL of sterile CAMHB was transferred to each well from well 2 to well 12. Next, 50 μL was removed from well 1 and added to well 2. The contents from well 2 were mixed by pipetting up and down. Next, 50 L were removed from well 2 and added to well 3. This process was continued till well 11 is reached. 50 μL was discarded from the well 11. Each compound concentration was tested in triplicate.

(c) Inoculation of 96-well plate: The same number of bacteria (1×10⁵ CFU/mL) were prepared after 150-fold dilution of 0.5 McFarland inoculum and used to add in the wells of different drug concentrations. 50 μL of working inoculum was carefully added to each well, except the sterility well 12. Mixing is performed by gently flushing the inoculum in and out of the pipette tip four or five times, avoiding splashing and bubble formation. Apart from the samples, three controls were used for the assay: (i) Growth control (GC), which have bacteria but no drug to show how bacteria grow without the compound; (ii) Sterility control (SC) in well 12, which doesn't contain any bacteria or drug and expected not to show any bacterial growth after incubation; and (iii) DMSO control (DC), which does not have drug to show the effect of DMSO on bacterial growth.

(d) Incubation: Plates were incubated at 35° C. for 24 hours without shaking or agitation.

(e) Determination of MIC: After 18-24 hours of incubation, the plates were read for MIC. SpectraMax iD5 microplate reader was used to measure turbidity at different time points. Turbidity (OD) increases as the bacteria grow over time. In general, the bacterial growth curve can be separated into different phases starting with lag phase, continuing with the exponential growth known as log phase after that depletion of nutrients is the one factor to limit bacteria growth in the stationary phase, which results in cell death later.

The OD values at a different time point show a typical growth curve in the growth control well, but no growth in the sterility well. Lower concentrations of compounds are not sufficient to prevent bacterial growth, but higher concentrations are sufficient to inhibit growth. The lowest concentration to inhibit the visible growth of bacterial growth is MIC for the compound.

(f) Determination of MBC: MBC was determined by sampling all the macroscopically clear wells. Before sampling, the wells were gently mixed by flushing them with a pipette, and a 5 μL was removed and placed on an agar plate suitable for the growth of the microbe being tested. The agar plate was divided into three parts to test three trials of a single concentration. The sample was allowed to be absorbed into the agar for ˜30 minutes. The plates were then incubated for 24 hours at 37° C. After incubation, the plates were examined to determine the compound concentration at which 99.9% killing was achieved.

### 2. Antibacterial Activity of Harmaline Dimer Compounds and Harmaline

The antibacterial activity of a set of novel harmaline dimer analogs, GK506.2Im, GK517.2, GK517.3, GK517.4, GK524, and GK585 was evaluated. Harmaline and GZ440/6 were also tested for antibacterial activity. At the tether, GK506.2Im has an imidazole ring; GK517.2, GK517.3, and GK517.4 have a pyridine ring with N at different positions; GK524 has a thiazole ring; and GK585 has trifluromethylpyridine, while GZ440/6 has no substitution. The comparative study helps to understand the role of substitutions at tether towards their antibacterial activity.

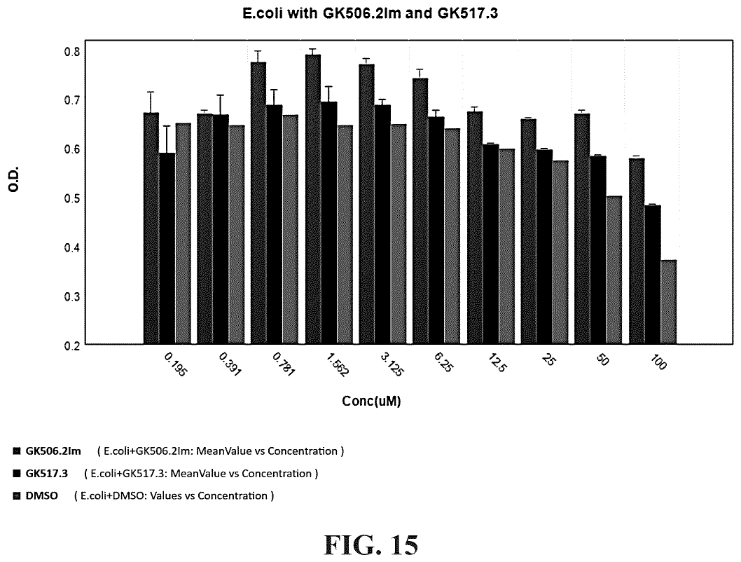

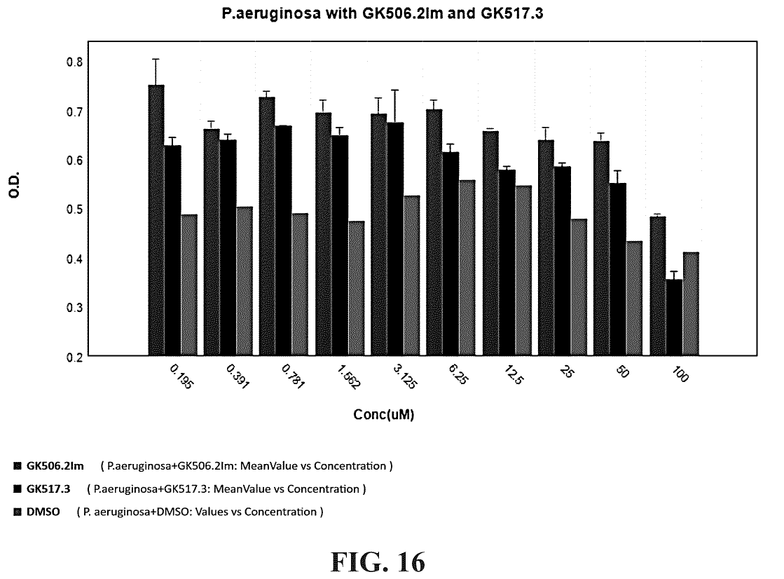

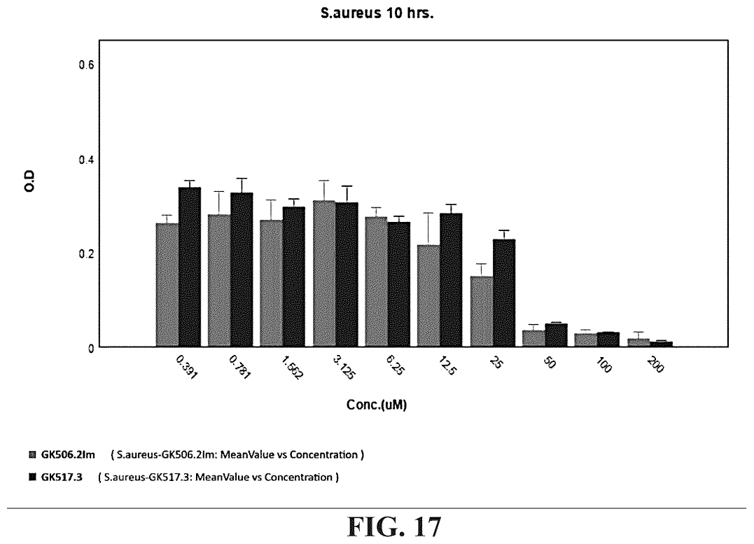

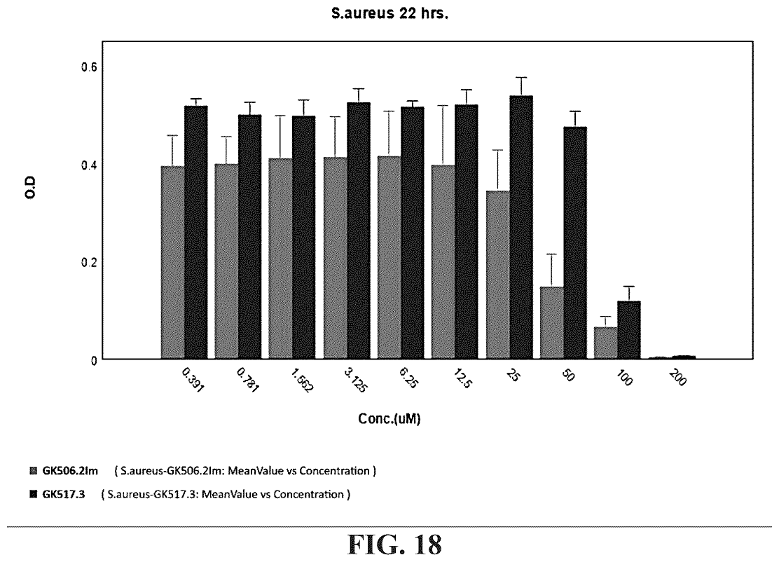

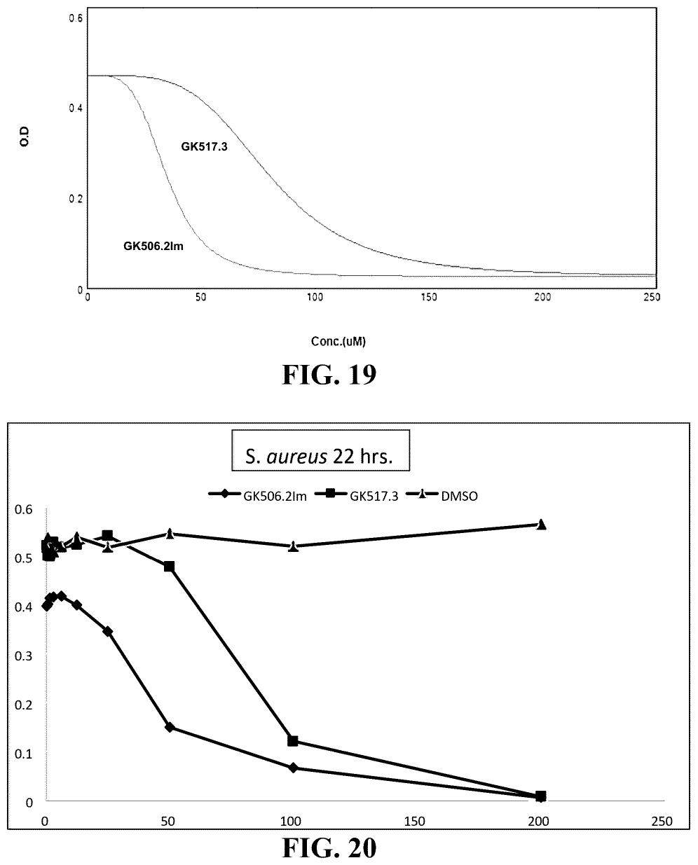

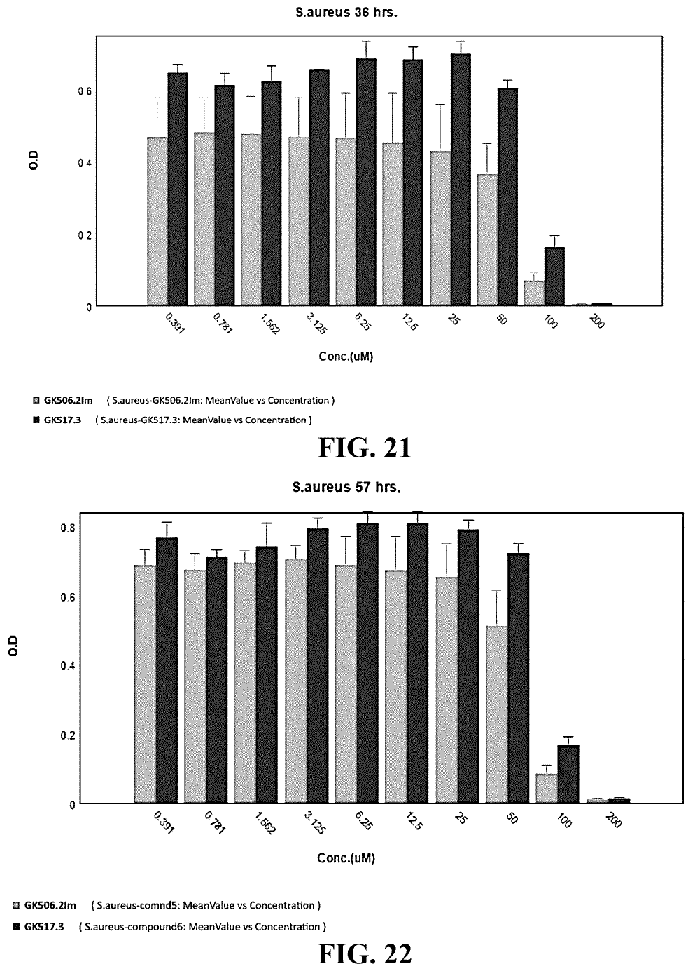

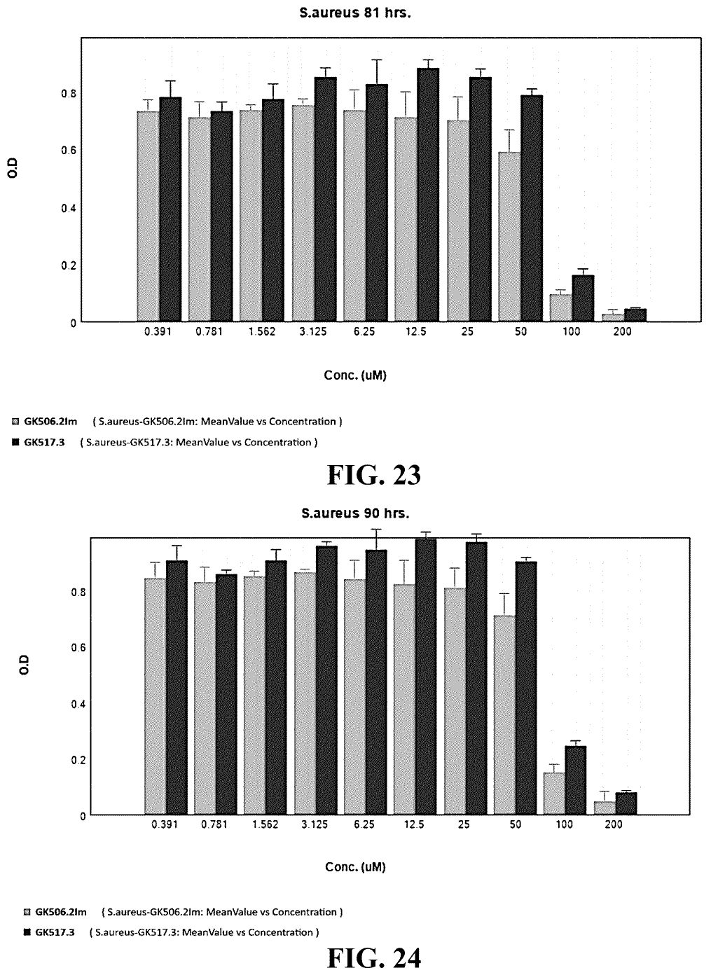

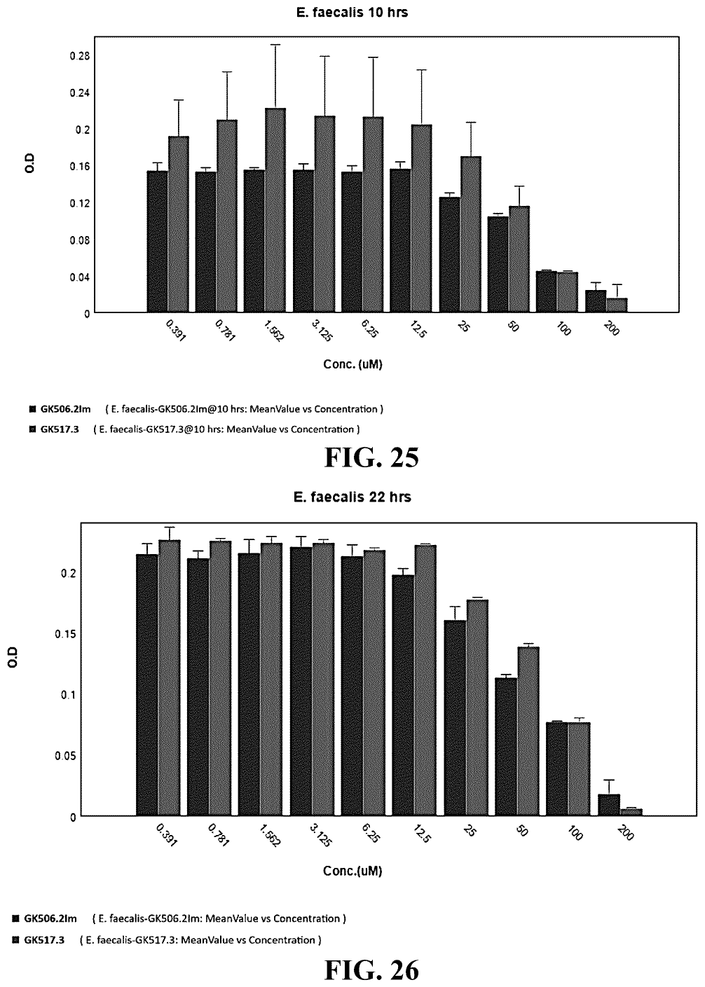

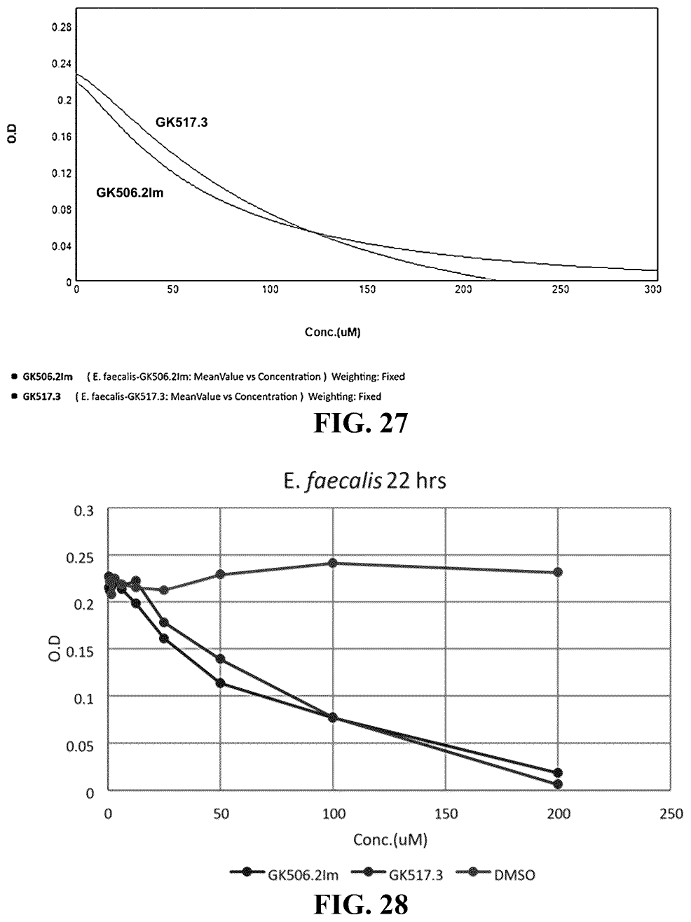

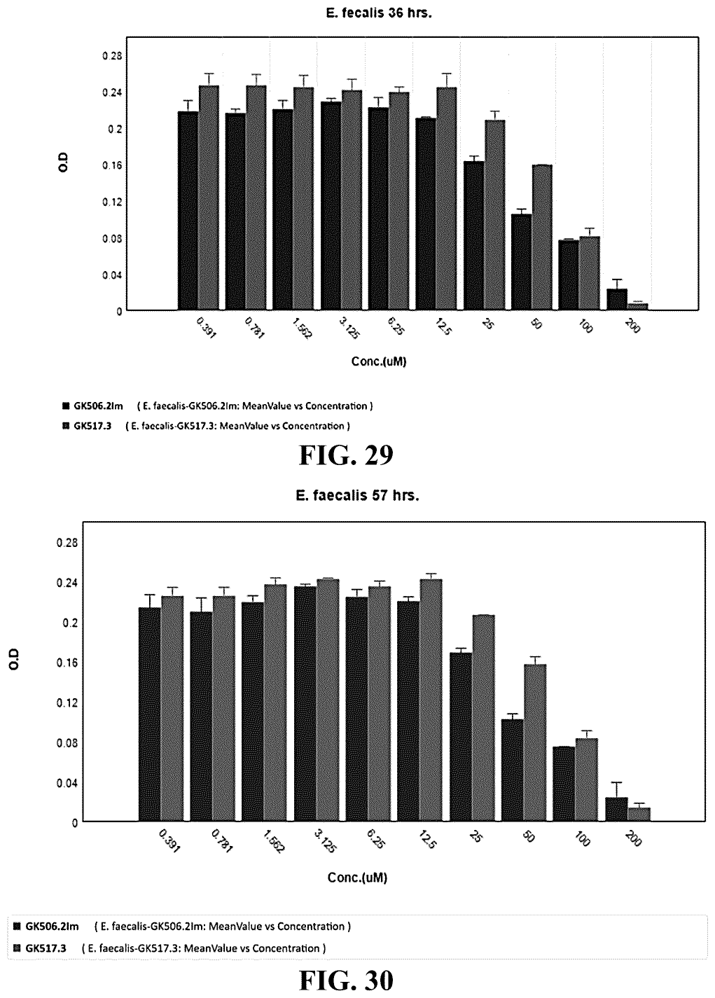

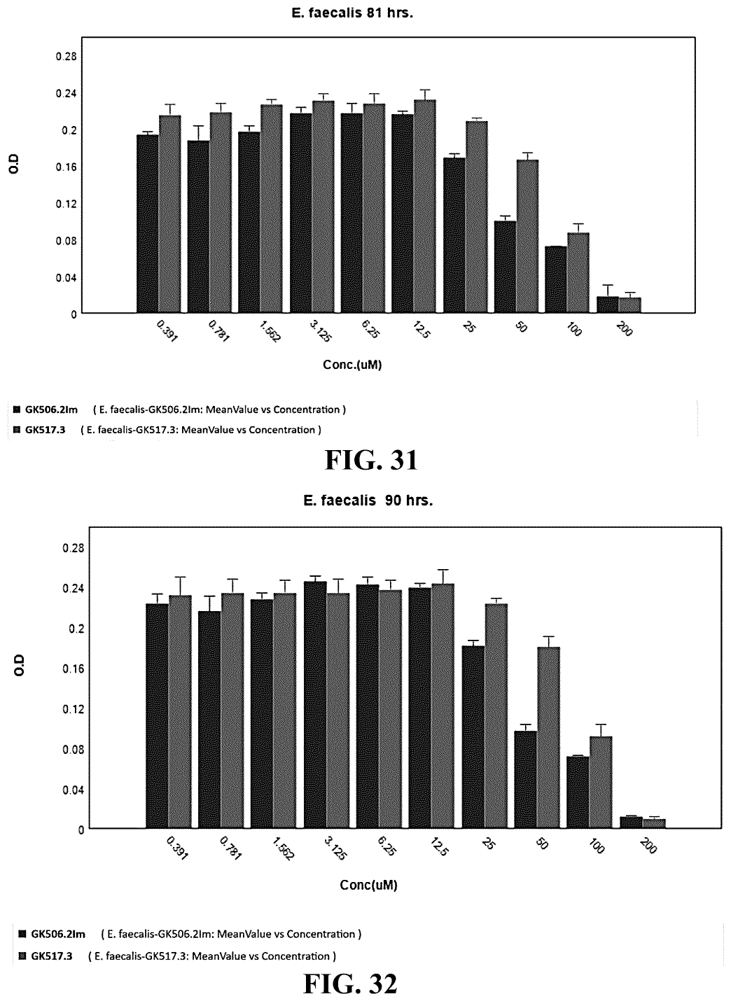

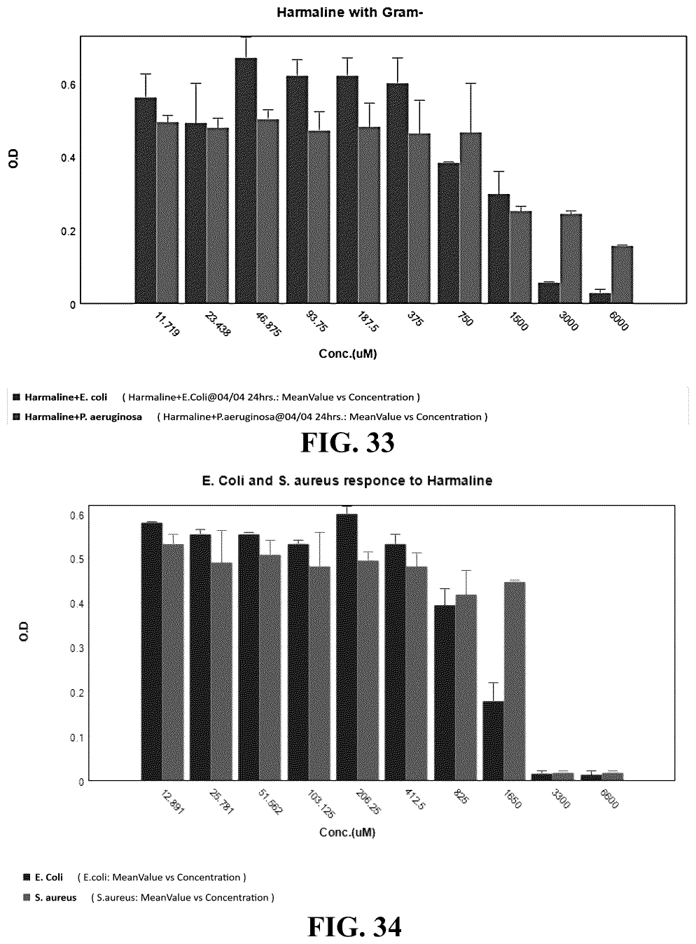

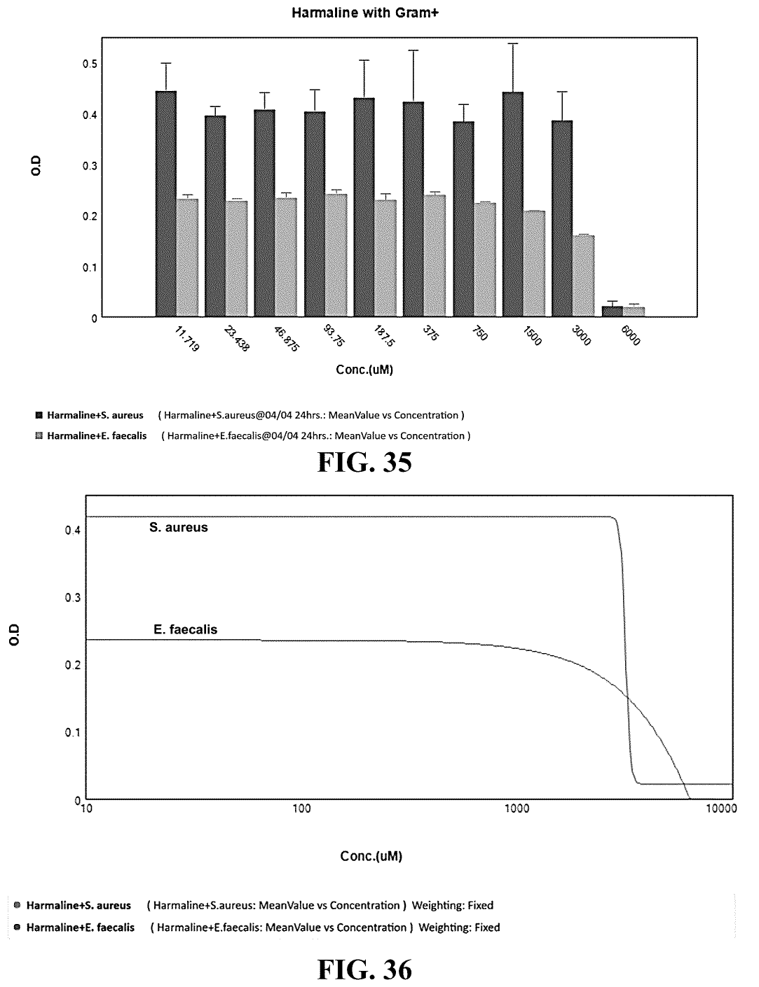

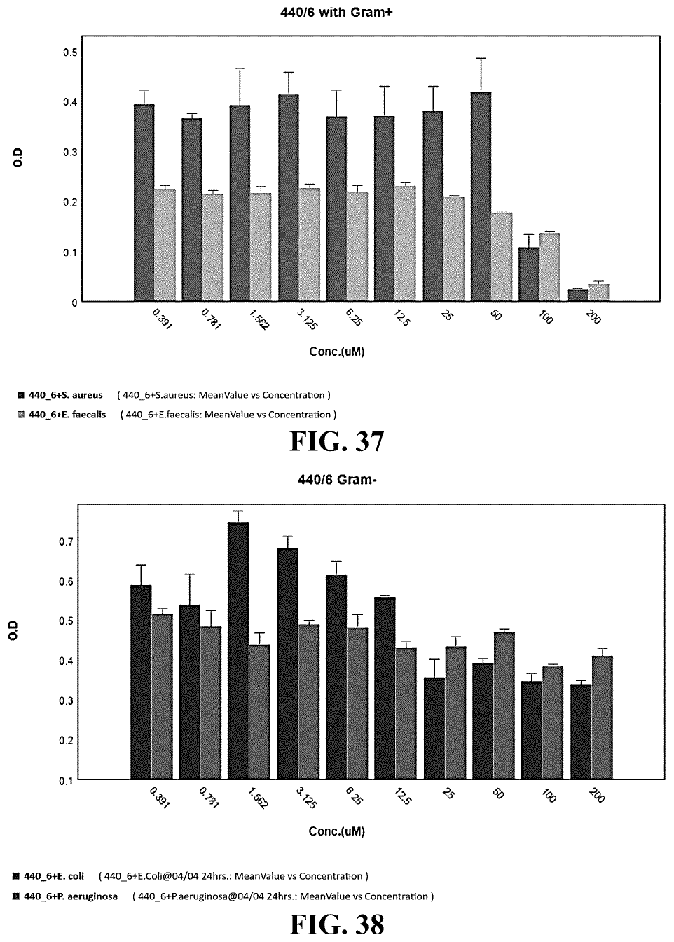

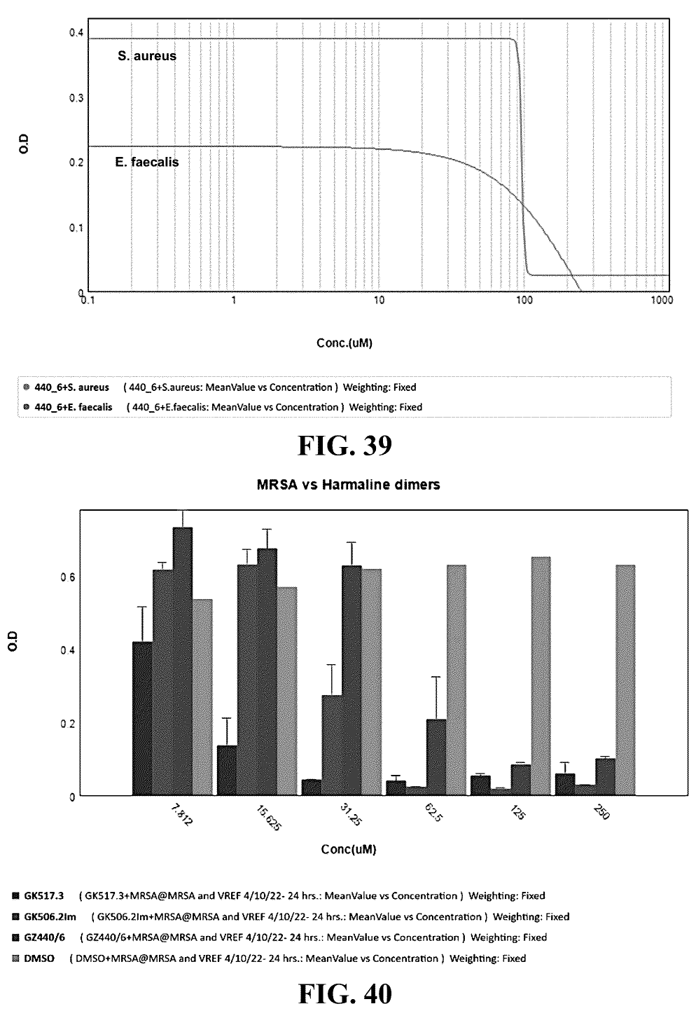

FIGS. 15 and 16 show the E. coli and P. aeruginosa response to GK506.2Im, GK517.3, and DMSO control; no inhibition was observed for these gram-negative bacteria at tested concentrations. However, GK506.2Im and GK517.3 compounds showed bacterial growth inhibition at 50.62 μg/mL and 51.7 μg/mL and killed bacteria at 101.2 μg/mL and 103.5 μg/mL (200 μM), respectively against gram-positive bacteria, S. aureus (see FIGS. 17-24) and E. faecalis (see FIGS. 25-32). Harmaline showed bacterial growth inhibition at a very high concentration, 707 μg/mL (3300 μM) against both E. coli and S. aureus (see FIGS. 34-36). Harmaline dimer, GZ440/6, displayed growth inhibition at 88 ug/mL (200 μM) against S. aureus and E. faecalis (see FIGS. 37 and 39). FIGS. 32 and 38 show the E. coli and P. aeruginosa response to harmaline and GZ440/6, respectively.

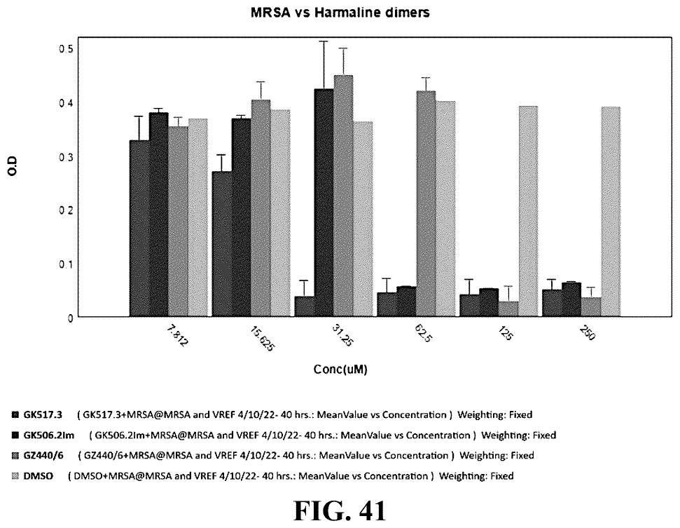

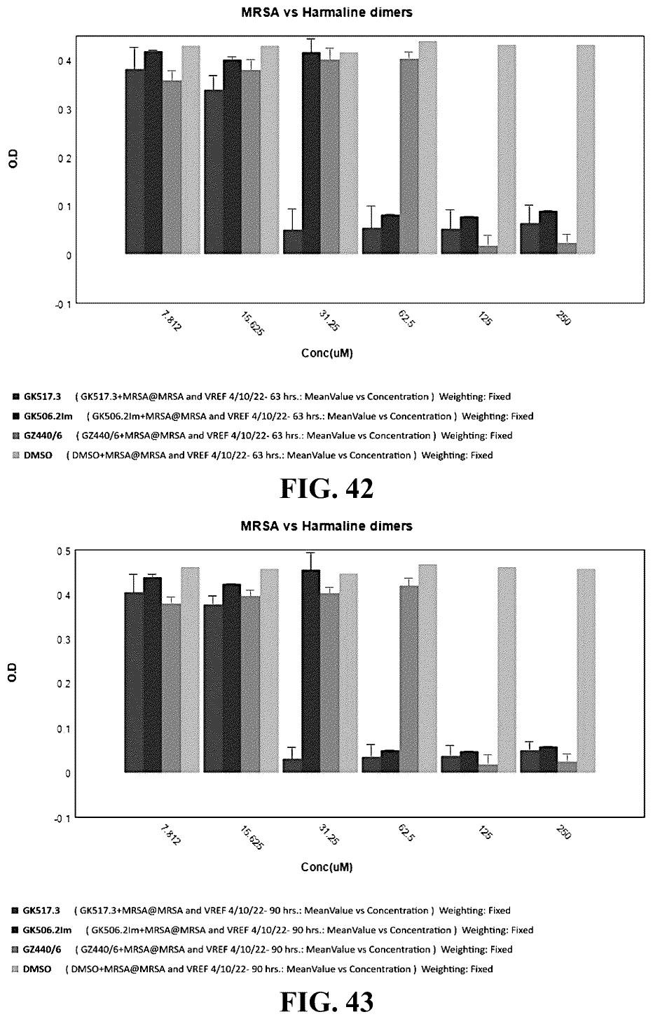

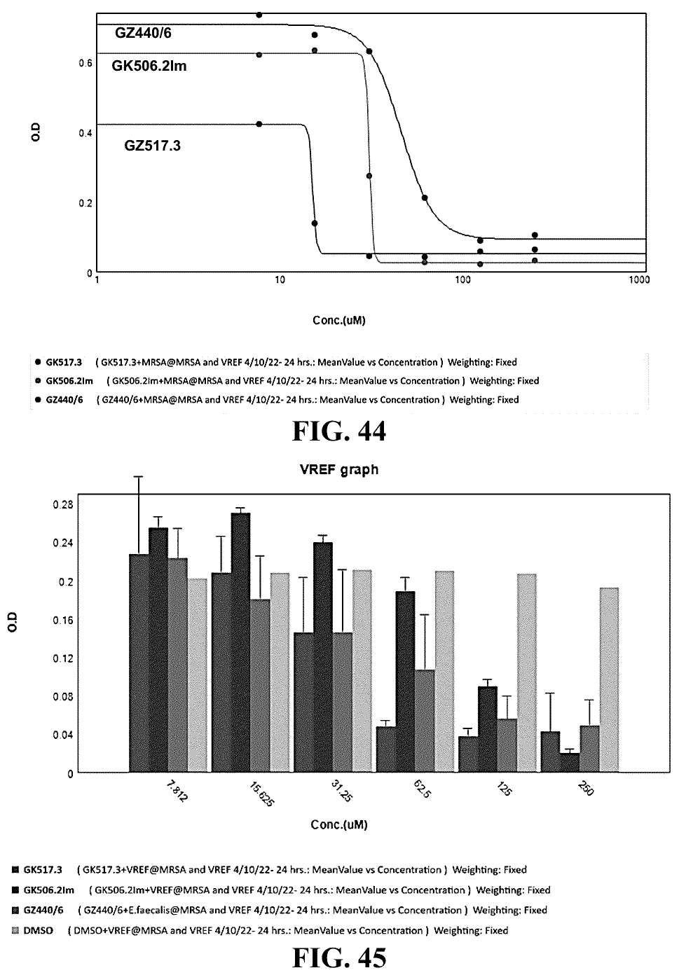

In the antibacterial screening with MRSA, GK517.3 showed bacterial growth inhibition at 8 μg/mL up to 24 hours (FIGS. 40, 44) and at 16.18 μg/mL up to 90 hours, and GK506.2Im shows bacterial growth inhibition at 15.83 μg/mL up to 24 hours (FIG. 40) and at 31.66 μg/mL up to 90 hours (see FIGS. 41-44). GK517.3 and GK506.2Im show bactericidal activity against MRSA at 32.35 μg/mL and 63.32 μg/mL, respectively. GZ440/6 shows bacterial growth inhibition at 27.53 μg/mL up to 24 hours (FIG. 40) and at 55 μg/mL up to 90 hours (see FIGS. 41-44). GZ440/6 has an MBC of 110.12 μg/mL.

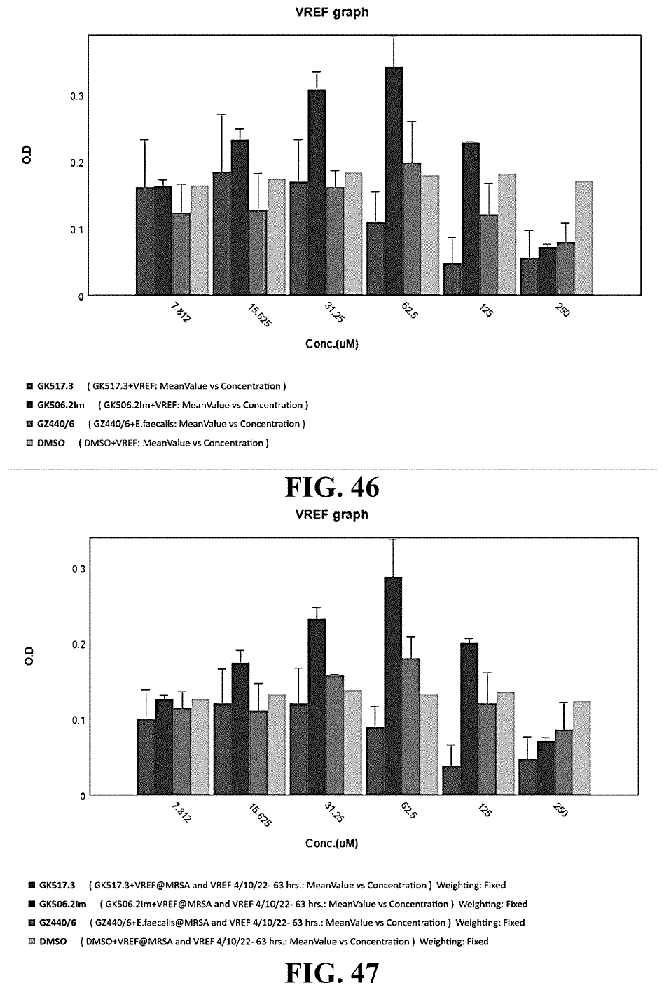

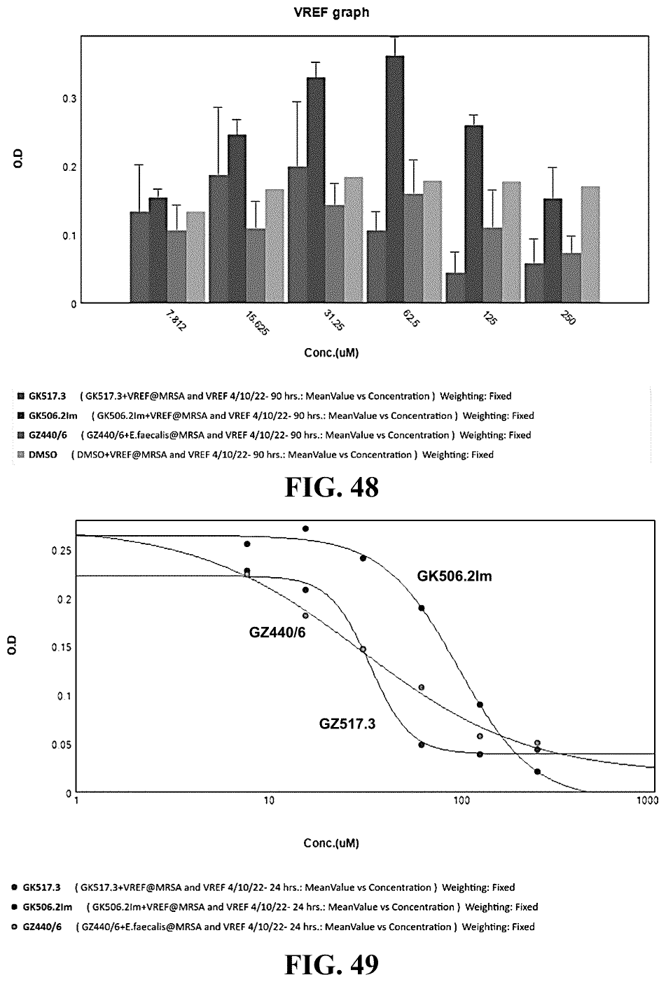

In the antibacterial screening with VREF (vancomycin resistant and teicoplanin sensitive E. faecalis), GK517.3 shows bacterial growth inhibition at 32.35 μg/mL up to 24 hours (FIG. 45) and at 64.7 μg/mL up to 90 hours (FIGS. 46-49), and GK506.2Im shows bacterial growth inhibition at 63.3 μg/mL up to 24 hours, at 126.6 μg/mL at 48 hours, and at >126.6 μg/mL at 63 and 90 hours. GK517.3 and GK506.2Im show bactericidal activity against VREF at 64.70 μg/mL and >126.65 g/mL, respectively. GZ440/6 shows bacterial growth inhibition at 55 μg/mL at 24 hours (FIG. 45), at 110 ug/mL at 48 hours, and at >110 ug/mL at 63 and 90 hours (FIGS. 46-49). No bactericidal action was observed for GZ440/6 at the concentrations tested (up to 250 PM).

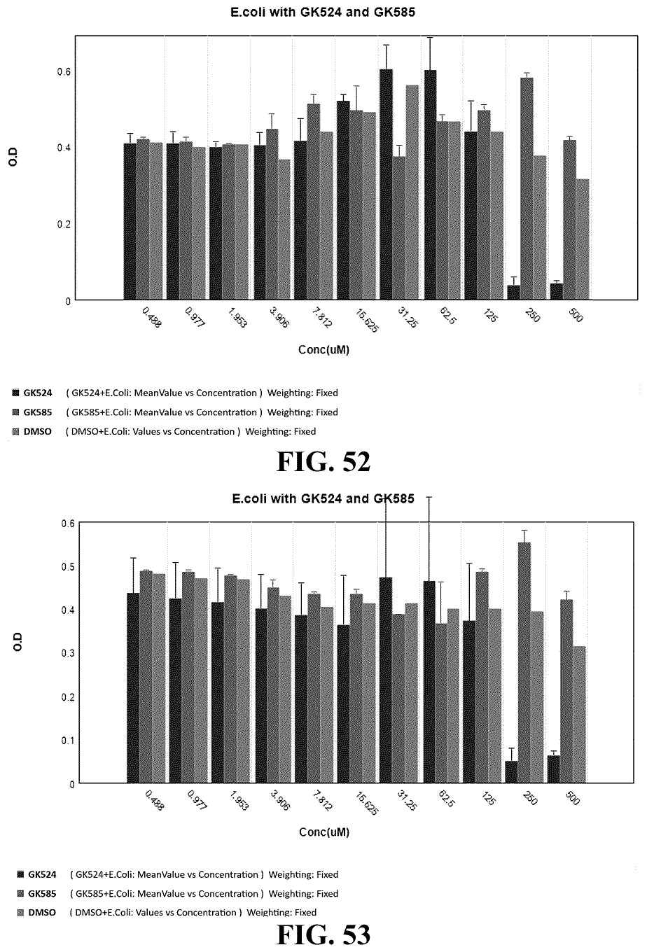

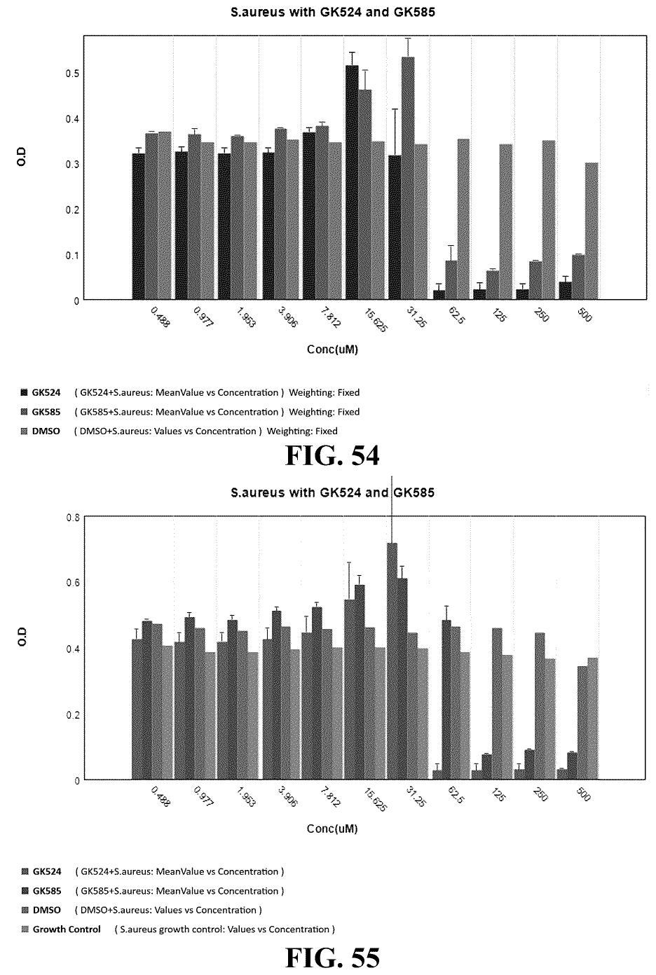

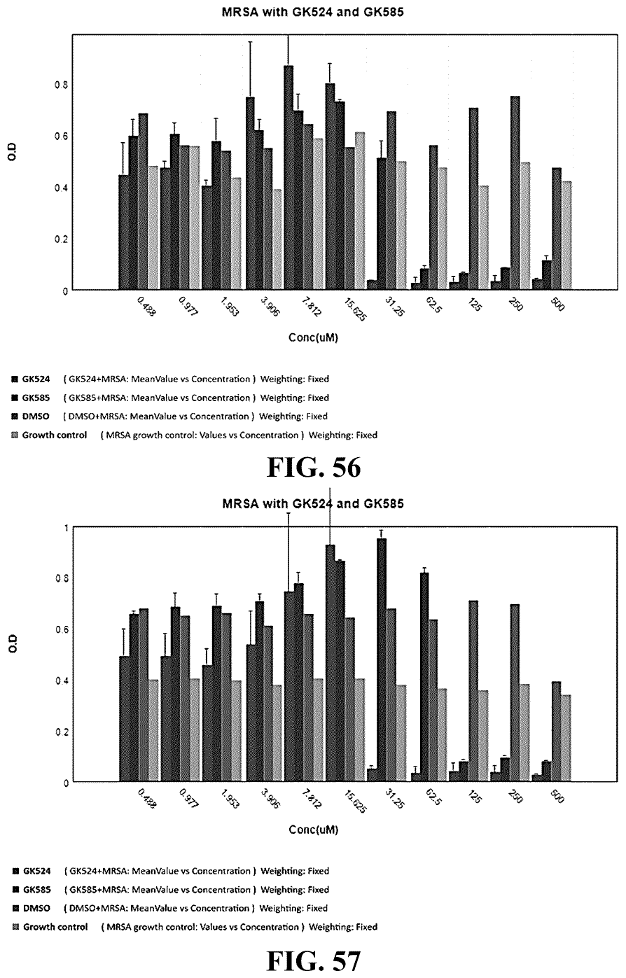

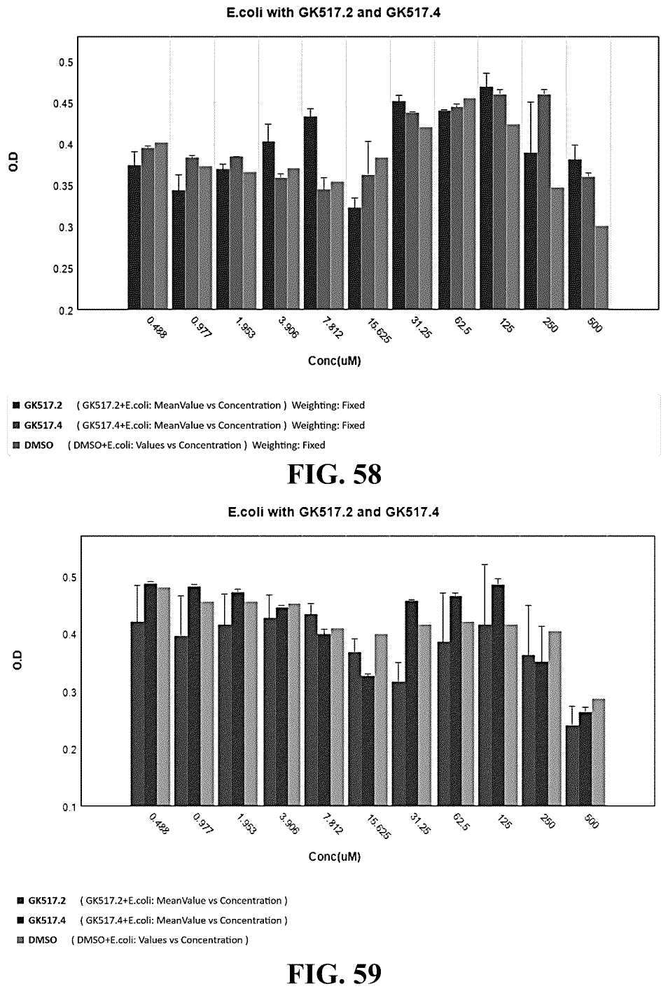

We also tested compounds GK524 and GK585 for antibacterial activity. Against S. aureus, GK524 showed bacterial growth inhibition at 32.72 μg/mL (FIGS. 54-55) and killed 99.9% bacteria at 65.45 μg/mL. Against MRSA, GK524 shows MIC at 16.36 μg/mL (FIGS. 56-57) and MBC at 32.72 μg/mL. However, GK585 shows MIC at 73.20 μg/mL and MBC at >146.40 μg/mL in S. aureus (FIGS. 54-55) and MRSA (FIGS. 56-57). FIGS. 52-53 shows the E. coli response to GK524 and GK585; No bacteriostatic or bactericidal activities were observed at the tested concentrations. We also tested compounds, GK517.2 and GK517.4, to see the effect of N-position in pyridine ring on their antibacterial activity. GK517.2 and GK517.4 inhibits bacterial growth at 64.70 μg/mL against S. aureus (FIGS. 60-61) and MRSA (FIGS. 62-63) up to 24 hours. Against S. aureus, GK517.2 and GK517.4 show MBC>129.40 μg/mL and 64.70 μg/mL, respectively. While, both GK517.2 and GK517.4 have MBC>129.40 μg/mL against MRSA. FIG. 60-61 show response of GK517.2 and GK517.4 on E. coli; no activity was observed at tested concentrations.

Data suggests that the harmaline dimer molecules (GK506.2Im, GK517.3 and GK524 compounds) show higher efficacy than harmaline by ˜15-fold. Harmaline dimer molecules (GK506.2Im, GK517.3, and GK524 compounds) with a substitution at the tether have 1.75-fold higher antibacterial activity than harmaline dimer without a substitution (GZ440/6). Compounds GK506.2Im and GK517.3 show bactericidal activity at 200 μM concentration for gram-positive bacteria S. aureus and E. faecalis, while GK524 shows bactericidal activity at 125 μM concentration against S. aureus.

### 3. Additional MIC and MBC Data

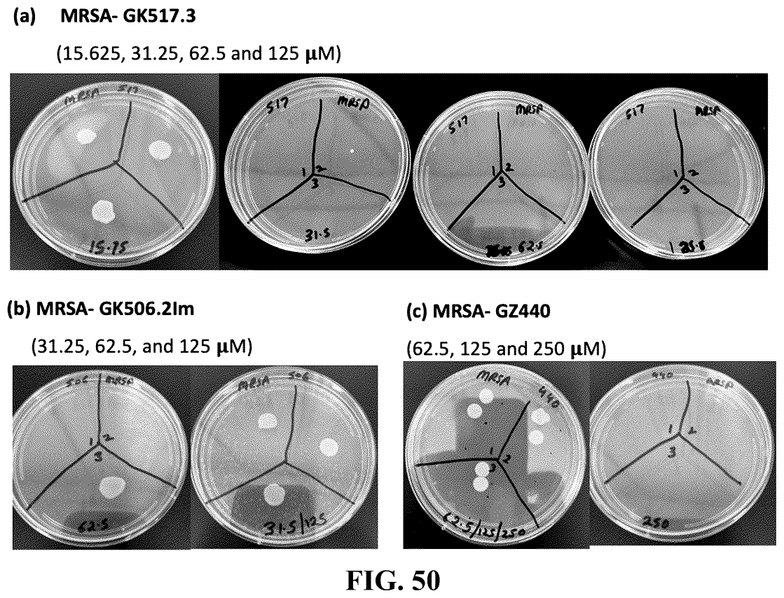

FIG. 50 shows images of MBC data of GK517.2, GK517.3, GK517.4, GK506.2Im, GK524, GK585, and GZ440/6 against MRSA, and this information is summarized in Table 3.

TABLE 3
MBC data of GK517.2, GK517.3, GK517.4, GK506.2Im, GK524,
GK585, and GZ440/6 against MRSA

| Compound/Bacteria strain | MRSA-MBC | |
|---|---|---|
| GK517.2 | >250 μM | (129.40 μg/mL) |
| GK517.3 | 62.5 μM | (32.35 μg/mL) |
| GK517.4 | >250 μM | (129.40 μg/mL) |
| GK506.2Im | 125 μM | (63.3 μg/mL) |
| GK524 | 62.5 μM | (32.72 μg/mL) |
| GK585 | >250 μM | (146.40 μg/mL) |
| GZ440/6 | 250 μM | (110 μg/mL) |

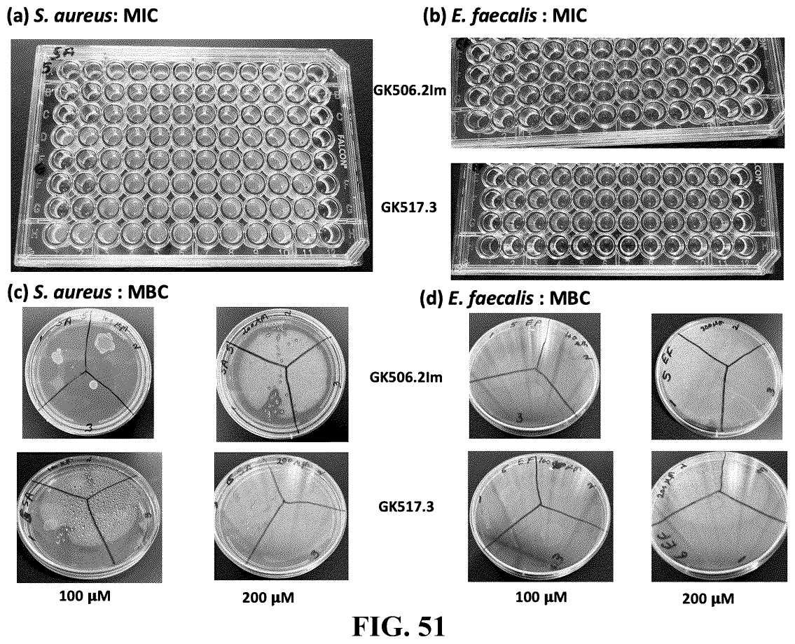

Furthermore, FIG. 51 shows MIC data and MBC data of GK517.3 and GK506.2 against S. aureus and E. faecalis.

In addition to the harmaline dimers compounds, other bicyclic indole compounds (203, 233, 476/6 and 504/6,) were also tested against gram positive and negative bacteria. Particularly, the indole ethyl urea molecules 203, 233 and the tryptamide dimer molecules 476/6, 504/6, as shown below, were tested.

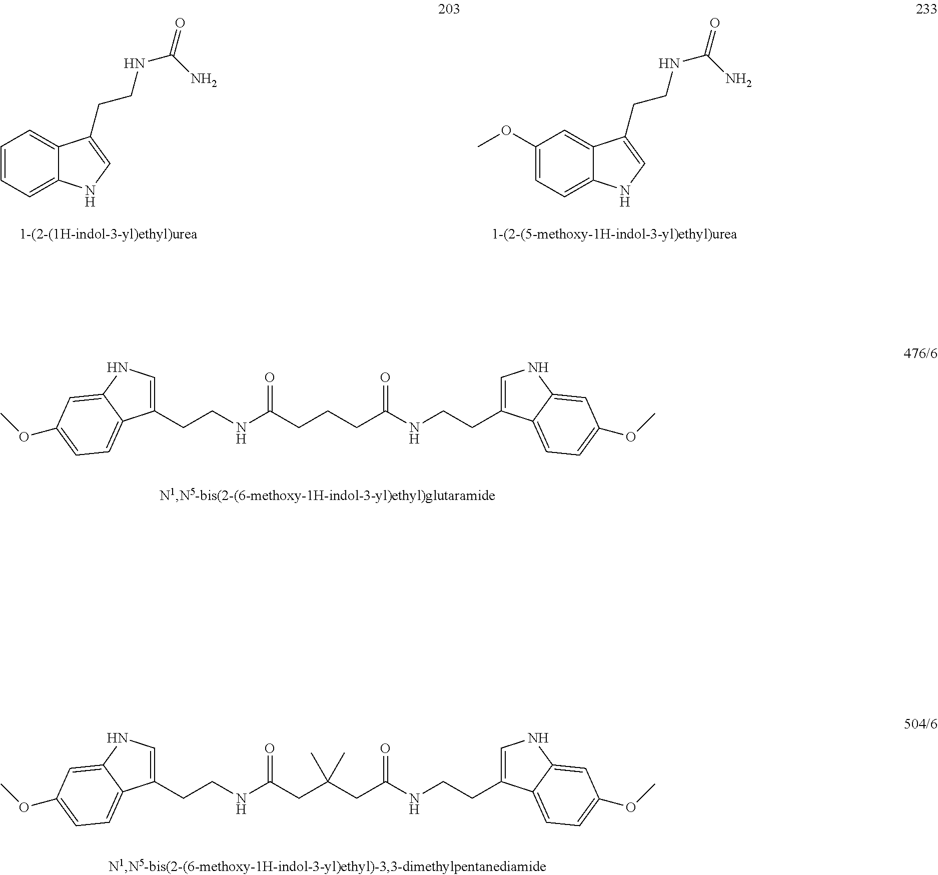

Notably, no bacteriostatic/bactericidal activity observed. There is no significant improvement in the activity of tryptamide dimers compared to indole urea compounds. The indole dimers (476/6, 504/6) showed no significant improvement in the activity compared to indole monomers (203, 233). In contrast, harmaline dimer molecules, such as GK517.3, GK506.2, GK524, and GZ440/6, show bacteriostatic and bactericidal activity, which demonstrates that tricyclic dimer molecules work better than bicyclic dimer (tryptamide dimer) molecules. Harmaline dimers show better activity than the harmaline monomer, and harmaline dimers with substitution at the tether show even better activity than the one without substitution. Specifically, the thiazole (GK524) and pyridine substitution with N at a specific position in ring (GK517.3) shows better activity than imidazole substitution at the tether. In addition, when a nitrogen replaced with sulfur atom in imdazole ring, the activity was improved. The results signify the importance of N and S atoms at a specific position in molecules GK517.3 and GK524 for better activity.

### Example 6

### In Vitro Assay of COVID-19 Using Harmaline Dimer Compound

The antiviral activity of a harmaline dimer compound, designated as GZ440/6, at different concentrations (2 μM, 5 μM, 10 μM, 20 μM and 40 μM) on coronaviruses was assessed in vitro using novel coronavirus SARS-CoV-2 isolate USA-WA1/2020 (deposited by the Centers for Disease Control and Prevention and obtained through BEI Resources, NIAID, NIH: SARS-Related Coronavirus 2, Isolate USA-WA1/2020, NR-52281) and human coronavirus strain HCoV_NL63 (obtained through BEI Resources, NIAID, NIH: Human Coronavirus, NL63, NR-470).

For propagation and experimentation with SARS-CoV-2, we used Vero E6 (ATCC® CRL-1586™) and Calu-3 cells (ATCC® HTB-55™) purchased from the American Type Culture Collection (ATCC, Manassas, VA, USA) and maintained in EMEM (Eagle's Minimum Essential Medium) cell culture media (cat #30-2003, ATCC, Manassas, VA, USA) supplemented with 2% or 10% fetal bovine serum (FBS), 100 U/mL penicillin, 100 μg/mL streptomycin, 0.01M HEPES buffer solution, 1 mM sodium pyruvate, 1× non-essential amino acids solution (cat #SH3023801, Thermo Fisher Scientific, Waltham, MA, USA), and 2 mM L-glutamine. For the propagation and experimentation with the NL63 coronavirus, we use LLC-MK2 cells (ATCC® CCL-7™) maintained in Medium 199 (cat #M4530, Millipore Sigma, St. Louis, MO, USA) supplemented with 2% or 10% FBS, 100 U/mL penicillin, and 100 μg/mL streptomycin.

Inhibition of viral replication was assessed using reverse-transcription quantitative real-time PCR (RT-qPCR) to measure the number of virions released into the cellular supernatant of infected cells and virus plaque formation assays, hereafter plaque assays, which measure a reduction in plaque forming units (PFUs). Cytotoxicity was measured using Promega™ CytoTox 96™ Nonradioactive Cytotoxicity Assay (cat #G1780, Promega, Madison, WI, USA).

A. Plaque Assays—Viral Titers

6-well plates (cat #CLS3516, Millipore Sigma, St. Louis, MO, USA) were seeded with ˜3.0×10\hat{ }5 cells/well. Vero E6 and LLC-MK2 cells were incubated for 48-72 hours at 37° C. in 5% CO₂ atmosphere until 80-90% confluency was achieved, while Calu-3 cells were incubated for >10 days to achieve 80-90% confluency. Prior to infection the 10% FBS containing growth media was replaced with fresh media containing 2% FBS supplemented with varying concentrations of GZ440/6 (as appropriate for each experiment (see results) and infected with coronavirus (SARS-CoV-2 or NL63) at an MOI of 0.013 for SARs-CoV-2 and 0.003 for NL63 and incubated at 37° C. for 1 hour. The virus and GZ440/6 containing media was then replaced with a 1×DMEM (cat #D2902, Millipore Sigma, St. Louis, MO, USA) agarose overlay containing the appropriate drug concentrations (10 μM, 20 μM and 40 μM) for each experiment, left to solidify at room temperature for 15 minutes, and incubated for 5 days at 37° C. in 5% CO₂ atmosphere. Combined camostat mesylate and E64d, hereafter “C/E”, was used as an inhibition control for all experiments (Zhan, 2020; Targeting the entry step of SARS-CoV-2: a promising therapeutic approach) and all SARS-CoV-2 processes were conducted in a biosafety level 3 facility.

After the 5-day incubation, 2 ml of 4% paraformaldehyde was added to each overlay and left to incubate for 30 minutes at room temperature to fix the cells and inactivate the virus. The cells were then stained with 1% crystal violet, the overlay was removed, and the cells were washed 3× with PBS. Plaque forming units for each treatment group were counted, averaged, and normalized to the untreated control group. Each experimental run contained three biological replicates and each experiment was conducted a minimum of two times. Standard deviation was calculated using the variation of averaged counts among all runs. Values were plotted using GraphPad Prism version 8.0.0 for Windows (GraphPad Software, San Diego, CA, USA) and annotations were added using Adobe Illustrator (Adobe Systems Incorporated, San Jose, CA, USA).

B. Real-Time (RT)-qPCR

Infection and Viral RNA Extraction

12-well plates (cat #CLS3513, Millipore Sigma, St. Louis, MO, USA) were seeded with ˜1.0×10\hat{ }5 cells/well. Vero E6 and LLC-MK2 cells were incubated for 48-72 hours at 37° C. in 5% CO₂ atmosphere until 80-90% confluency was achieved, while Calu-3 cells were incubated for >10 days to achieve 80-90% confluency. Prior to infection, the growth media was replaced with fresh media containing 2% FBS supplemented with varying concentrations of GZ440/6 as appropriate for each experiment (see results) and infected with coronavirus (SARS-CoV-2 or NL63) at an MOI of 0.04 for SARs-CoV-2 and 0.01 for NL63 and incubated for up to 5 days at 37° C. in 5% CO₂ atmosphere. 1× Camostat/E64D was used as an inhibition control for all experiments and all SARS-CoV-2 processes were conducted in a biosafety lab level 3 facility. Each experimental run contained two biological replicates and each experiment was conducted a minimum of two times. 400 μL of supernatant was harvested at 48 hours for SARS-CoV-2 but 5 days for NL63 and RNA was extracted using Invitrogen Pure-Link RNA kits (Thermo Fisher Scientific, Waltham, MA, USA) following the manufacturer's recommendations for liquid extractions.

qPCR Assay Design

Two TaqMan qPCR assays were designed to target and amplify either the SARS-CoV-2 or the NL63 coronavirus. The SARS-CoV-2 qPCR amplified a 125 bp region of the spike protein (GenBank Gene ID: 43740568, incorporated by reference herein) using forward primer CoV2-S_19F (5′-GCTGAACATGTCAACAACTC-3′ (SEQ ID NO:1)) and reverse primer CoV2-S_143R (5′-GCAATGATGGATTGACTAGC-3′(SEQ ID NO:2)) with MGB TaqMan probe CoV2-S_93FP (5′-ACTAATTCTCCTCGGCGGGC-3′(SEQ ID NO:3)) labelled fluorescently with a FAM dye, which was designed based off the SARS-CoV-2 genome GCF_009858895.2 (GenBank Sequence ID: MN908947.3), while the NL63 qPCR amplified a 201 bp region of membrane protein (GenBank Gene ID: YP_003770.1) using forward primer NL63_10F (5′-TGGTCGCTCTGTTAATGAAA-3′(SEQ ID NO:4)) and reverse primer NL63_200R (5′-AAATTTCTTCCTAGCAGCTC-3′(SEQ ID NO:5)) with MGB TaqMan probe NL63_102RP (5′-CCCTCCTGAGAGGCAACACC-3′(SEQ ID NO:6)) fluorescently labelled with a VIC dye, which was based off the HCoV_NL63 genome (GenBank Sequence ID: MN306040.1, incorporated by reference herein).

Reverse Transcription and PCR Amplification

Two approaches were used over the course of this study for the reverse transcription of RNA into cDNA followed by qPCR. For the first approach we used a two-step method where viral RNA was converted into cDNA using Invitrogen SuperScript IV VILO Master Mix (cat #11766500, Thermo Fisher Scientific, Waltham, MA, USA) in 96-well format according to the manufacturer's recommendations and deactivated using 1 μL of Invitrogen RNase H (cat #18021-014, Thermo Fisher Scientific, Waltham, MA, USA); these processes used a SimpliAmp® thermocycler (Applied Biosystems, Foster City, CA, USA). 1 μL of template cDNA was then subjected to qPCR in 10 μL reactions containing 1× TaqMan Universal Master Mix II (w/o AmpERASE UNG) (Applied Biosystems, Foster City, CA, USA), with 0.2 μM of each forward and reverse primer and 0.1p M of probe for the SARS-CoV-2 qPCR and 0.25p M of each forward and reverse primer and 0.125 μM of probe for the NL63 qPCR, and thermocycled in triplicate reactions using either a QuantStudio 7 flex or QuantStudio 12K (Applied Biosystems, Foster City, CA, USA) under the following conditions: 10 minutes at 95° C., proceeded by 40 cycles of 95° C. for 15 seconds and 60° C. for 1 minute. The second approach employed a one-step procedure in which viral RNA was converted to cDNA using the TaqMan amplification primers, immediately followed by qPCR. For this approach we used the 4× Reliance One-Step Multiplex RT-qPCR Supermix (cat #12010220, BioRad, Hercules, CA, USA) with the same TaqMan primers, probes, and concentrations as described above for the two-step approach; however, these reactions were conducted in 20 μL volumes, instead of 10 μL. Triplicate reactions were thermocycled using the same QuantStudio instruments described previously under the following conditions: 50° C. for 10 minutes to reverse transcribe the viral RNA into cDNA, followed by 95° C. for 10 minutes, proceeded by 40 cycles of 95° C. for 10 seconds and 60° C. for 30 sec. Positive amplification and non-template controls were included on every run.

Data Analysis

A synthetic double stranded DNA fragment was generated (gBlocks Gene Fragments, Integrated DNA Technologies, Coralville, IA, USA) as a qPCR control that contained the amplification primers and internal gene sequence for both the SARS-CoV-2 and the NL63 target genes (see qPCR assay design section), of which copy number was known. By making a serial dilution of this control oligo from 10⁸ (undiluted) to 10′ copies and including it on every run, we were able to extrapolate viral copy number in each of the experimental samples. Based on the inclusion of this predefined synthetic gBlock standard the QuantStudio instrument software generated a standard curve for each run that was used to quantify the unknown sample reactions. The calculated quantities for triplicate reactions for each sample were averaged and the standard deviation was calculated among reactions. We performed a t-test to calculate the statistical significance of each experimental value compared to the untreated control group (data not shown). Values for the experimental replicates and the standard deviations among experimental runs were averaged and then normalized to the untreated control group to obtain percent inhibition values. These values were plotted using GraphPad Prism version 8.0.0 for Windows (GraphPad Software, San Diego, CA, USA) and annotations were added using Adobe Illustrator (Adobe Systems Incorporated, San Jose, CA, USA).

C. Cytotoxicity

Cytotoxicity was measured using the Promega™ CytoTox 96™ Nonradioactive Cytotoxicity Assay kit (cat #G1780, Promega, Madison, WI, USA) in 50 μL reactions and 96-well format (cat #161093, Thermo Fisher Scientific, Waltham, MA, USA) according to the manufacturer's protocol, except we used 20 μL of 10× Lysis Solution per 100 μL of sample and incubated at 37° C. for 30 minutes to generate the maximum LDH release control. The assay was measured using a BioTeK Synergy™ HT plate reader (model #7091000, BioTek, Winooski, VT, USA) at an optical density of 490 nm (OD₄₉₀). Cytotoxicity was measured for <60 μM Cmpd. x for all three cell lines (LLC-MK2, Vero E6, and Calu-3). LLC-MK2 cells were assessed for cytotoxicity at 5 days, whereas Vero E6 and Calu-3 cells were assessed for cytotoxicity every 24 hours from 1-5 days. Percent cytotoxicity was calculated by dividing the experimental LDH release at OD₄₉₀ b.

D. Results

Cytotoxicity Assay

There is no significant cytotoxicity in both lower (2, 5 and 10 μM) and higher doses (20 and 40 μM) of GZ440/6 as shown in FIGS. 64A-D, indicating GZ440/6 is not toxic to cells. There was negligible cytotoxicity observed in Vero-E6 cells treated with GZ440/6 at 2.0 μM, 5.0 μM and 10.0 μM (FIGS. 64A and 64B). There was also no significant cytotoxicity when the compound was administered at 20 μM and 40 μM (FIG. 64C). (Student's T-Test results GZ440/6 vs. Camostat/E64d P<0.05).

Virus Plaque Formation Assay

The virus plaque formation assay measures the efficiency of virus intercellular spread and induction of the cytopathic effect. This experiment uses live virus to infect mammalian cells. Virus was diluted to ˜500 plaque forming units/mL with growth medium (EMEM+2.0% Fetal Bovine Serum (FBS)). Cells were infected with virus for 1 hr. at 37° C., washed once with balanced salts solution, and fresh medium containing 2.0% FBS added. The cells were observed for the formation of cytopathic effects (CPE) and plaques (clear regions in cell monolayer caused by cell death) for 5 days. Viral RNA was also isolated for determination of virus copy number 5 days following treatment. In the typical intracellular lifecycle, the virus enters the cell, is uncoated (sheds its membrane), and releases nucleic acid into the cell cytoplasm. There, the virus makes copies of its genome, and packages these into new membrane vesicles which leave the cell by “budding”. The new virions then infect adjacent cells and the process continues as the virus infects new cells. This hijacking of the cellular machinery causes cellular damage (the cytopathic effect), which can be visualized as a zone of dead cells (i.e., a plaque). If a compound is effective, it may inhibit entry of the virus into the cells, multiplication or spreading from cell to cell. Plaquing efficiency for an effective inhibitor will be significantly lower than those for the untreated controls and may be comparable to the inhibitor (“C/E”) control. If this is not the case, then the compound does not inhibit the lifecycle of the virus under these conditions.

The assay was performed three times. Two representative charts are shown in FIG. 65A-65D. The results from the virus plaque formation assay using GZ440/6 at 2.0, 5.0 and 10.0 μM demonstrated negligible inhibition (FIG. 65A-65B). In FIG. 65A, the y-axis is a measure of the number of plaques of virus across each of the three treatment groups and two controls vs. untreated cells. Virus was diluted 100-200 plaque forming units (pfu) per well. FIG. 65B shows a measure of inhibitory activity relative to untreated controls. This is another way to look at the data. The Y axis is the degree of inhibition (measured in fold-differences) vs. untreated cells. Error bars are the standard deviation. In the presence of GZ440/6 at 2.0 μM, 5.0 μM and 10.0 μM, there was negligible inhibition in CoV-2 in Vero-E6 cells at 120 h after infection. However, at 20 μM and above, inhibition of plaque formation was significant (Student's T-Test results GZ440/6 vs. Camostat/E64d P<0.05) as shown in FIG. 65C-65D.

RT-PCR Assay

In parallel with the virus plaque formation assay, viral RNA was extracted from the cell supernatants at 5 days after infection with SARS-CoV-2. RNA levels indicate the number of copies of the virus measured by reverse transcription-polymerase chain reaction, or RT-PCR. If the treatment is inhibitory, RNA levels will be significantly lower in the treated vs. the untreated control. If treatment is not inhibitory, then the RNA copy number will be more comparable to the untreated controls. Viral RNA extractions were assayed in duplicate for each treatment per run of the virus plaque formation assay. The experiment was performed at least 3 times. Overall, the RT-PCR assay has a greater range of detection and sensitivity compared to the plaque assay above (FIGS. 65A-65D). Quantitation of viral copy number by RNA measurements indicated consistent reduction of viral numbers in cells 120 hours after viral infection to cells. Treatment of GZ440/6 at higher concentrations (20 and 40 μM) decreased viral load indicating reduction of viral RNA levels in higher concentrations, but not in lower concentrations as shown in FIG. 66A-C. These results are consistent with plaque formation as demonstrated in FIG. 65A-D.

### Example 7

### Genotoxicity of GZ440/6, GK506.2Im, GK524, GK517.3, and GK585

### 1. Genotoxicity Protocols

Genotoxic compounds are harmful due to their ability to react negatively with cellular DNA and induce mutations or other physical damage that alters protein production and cellular function. The EBPI UMU-ChromoTest™ assay is used for the detection and quantification of genotoxicity of compounds and is based on the International Organization for Standardization protocol ISO 13829. The UMU-ChromoTest™ assay utilizes a native bacterial mechanism for the detection of compound genotoxicity. The bacteria used for the assay is an engineered Salmonella typhimurium strain developed to include a gene coding for the β-galactosidase (β-gal) enzyme tethered to the umuC, a damage response gene. DNA damage activates the SOS system to transcribe β-gal proportional to the level of SOS induction. The UMU-ChromoTest™ bacterial strain has modifications to increase sensitivity to genotoxins with (a) increased sensitivity for even limited damage, (b) modified outer membrane of the cell to increase permeability to exogenous materials, and (c) production of a color change on addition to chromogenic substrate (ONPG), which is a quantitative measure of genotoxic damage to bacterial DNA.

The test has the following steps:

1. Rehydration of Dried Bacteria and Culture Growth: The bacteria were rehydrated and incubated at 37° C. for 16-18 hours. OD₆₀₀ of the overnight culture should be 0.5 or at least above 0.1 at 600 nm.

2. Sample Preparation and Plate Setup: The dimer compounds were dissolved in DMSO. All dilution and controls were made in the solvent of 10% DMSO in sterile 0.85% saline. The pH of the compounds was adjusted to 7.0±0.2 before incubation with bacteria to facilitate bacterial growth and prevent degradation of ONPG later in the assay. The solutions of hydrochloric acid and sodium hydroxide were used to adjust pH with minimal addition volumes to prevent excessive sample dilution. Six samples were run at 4 dilutions at a time.

3. Preliminary Dilution of Bacteria: The overnight bacterial culture was diluted to 50% by performing a reinoculation with 5 mL of overnight growth and 5 mL of fresh Growth media. The reinoculated bacteria was inoculated for 1.5 hours at 37° C. The OD₆₀₀ should be approximately 80% of the reading from overnight growth or slightly higher.

4. Preparation of Plate A: Distilled water (180 μL) was added to all wells, except for rows A to F, columns 1 to 3 and row H, columns 1 to 6. Rows A to F are used for 6 compounds per plate. The test compounds (360 μL) were added to the first three wells (three replicates) of the same microplate, such that wells 1-3 were a triplicate set of the highest concertation of test compound (360 μL). Wells 4-12 each contained 180 μL of distilled water. For dilution, half of the contents (180 μL) from wells 1-3 was transferred to wells 4-6 which makes a second triplicate set. For a third dilution, half of the contents from wells 4-6 were transferred to 7-9, and for the fourth triplicate set, half of the contents from wells 7-9 was transferred to 10-12. This whole process makes triplicates of four different concentrations of the compound. 180 μL was discarded from the last three wells. 153 μL of water was added to the positive control and solvent control wells (H1-H6). 27 μL of a 10% water/DMSO solution was added to the solvent control to wells (H4-H6). 27 μL of 4-NQO was used as a positive control in wells H1-H3. 60 μL of 10× glucose supplemented Growth media was added to all wells. 70 μL of growth media was added to H7-H12 as solvent control.

5. Preparation and Incubation of Plate A: After 1.5 hours incubation of reinoculated bacteria (OD₆₀₀>80% of overnight value), 70 μL of the inoculum was added to wells (A to F, 1 to 12). 70 μL of inoculum was also added to the negative, positive, and solvent control excluding wells H7 to H12. Plate A was incubated at 37° C. for 2 hours.

6. Preparation and Incubation of Plate B: 270 μL of Growth media supplemented with 1× Glucose was added to all wells and brought to 37° C. in the incubator. 30 μL from each well of Plate A was added into the corresponding wells of Plate B to perform 10-fold dilution. The absorbance of Plate B was measured at 600 nm±20 nm before incubation to calculate bacterial growth. The plate B was incubated for 2 hours at 37° C. Near the end of incubation, the buffer was brought to room temperature and mixed with 30 μL of 2-mercaptoethanol. Phosphate buffer was added to the ONPG powder and mixed well. After 2 hours incubation, bacterial growth in Plate B was measured at 600±20 nm to calculate growth control.

7. Preparation and Incubation of Plate C: 120 μL buffer was added to each well of Plate C. 30 μL from each well of Plate B was transferred in the corresponding wells of Plate C. 30 μL ONPG solution was immediately added to all wells of Plate C and incubated for 30 minutes immediately. On development of yellow color, 120 μL of Stop solution was added to each well to intensify color development and aid in resolution. The absorption of solution was measured in the fully developed plate at 420±20 nm.

8. Color Development of the UMU-ChromoTest™: During the incubation period, genotoxic compounds interact with the DNA of modified bacteria and induce the de novo synthesis of β-galactosidase. At the last stage of UMU-ChromoTest™, relative amounts of β-galactosidase enzyme, produced because of the latter interaction, is measured by the addition of a chromogenic substrate, ONPG.

Analysis of the Results: The positive control (4-NQO) must show color development for the validation of the test. Results are visually analyzed for the yellow color density appeared in the compound wells starting from the highest concentration to the lowest in comparison with the solvent-only wells. The readings of wells were used to calculate the UMU Induction ration (UMU IR), an internationally accepted measure of genotoxicity. The induction ration was calculated by comparing a growth factor using absorbance values at OD₆₀₀ and β-gal activity from OD₄₂₀ measurements. For a genotoxic compound, the induction ration must be greater than 1.5 and the growth factor greater than 0.5. The positive control (4-NQO) showed color development which validates the test.

### 2. Genotoxicity Results

FIG. 67 shows the UMU response data for newly synthesized compounds. For a genotoxic compound, the induction ration must be greater than 1.5 and the growth factor greater than 0.5. All tested compounds (except GK585) did not show genotoxicity with UMU IR value<1.5 at minimum 85 μM concentration. GK585 shows no genotoxicity at 12.5 and 25 μM, however compound interfered with testing protocols at 50 and 100 μM concentrations.

### Example 8

Genotoxicity of GK506.2Im, GK517.3, and GK524 with and without S9 Activation

### 1. Genotoxicity Protocols

An UMU-ChromoTest assay was used to test the genotoxicity of compounds GK506.2Im, GK517.3 and GK524 (prepared according to the methods described in Examples 2-3, then recrystallized and dried overnight on vacuum to remove any residual impurities) with and without S9 activation (S9 is a crude liver enzyme extracted from Aroclor 1254-indused Sprague Dawley rats). The assay was conducted using the genotoxicity protocols described in Part 1 of Example 7.

### 2. Genotoxicity Results

Genotoxicity data suggest that GK524 exhibits lowest toxicity in comparison with GK517.3 and GK506.2Im. High genotoxic activity of GK506.2Im illustrates its ability to interact with the host DNA. All three compounds in metabolite form exhibit high genotoxicity (see FIG. 68) compared to their original form (see FIG. 69).

GK524 shows better activity on Gram positive bacteria compared to GK517.3 and GK506.2Im. GK524 also has high solubility in DMSO (instantly) compared to GK517.3 (10-15 mins) and GK506.2Im (30-40 mins). The antibacterial activity of GK524 enhanced in comparison with GK506.2Im, which illustrates the importance of sulfur for improved activity and solubility. Statistics: The following statistics has been calculated based on the readings obtained from SoftMax Pro 7.1.2 on SpectraMax ID5.

| | Without S9 | with S9 |
|---|---|---|
| Test validity (QC) Summary | activation | activation |
| Coefficient of Variation | 5.00 | 7.22 |
| of Negative Control (≤20%) | | |
| UMU IR value of Positive | 2.50 | 2.60 |
| Control (≥2) | | |
| Growth of Blank (≤0.100) | 0.09 | 0.09 |

### Example 9

GK580 Synthesis (3-(1,3-bis(7-methoxy-4,9-dihydro-3H-pyrido[3,4-b]indol-1-yl)propan-2-yl)-1H-indole-5-carbonitrile)

The reaction scheme is shown in FIG. 70. 0.99 g of Harmaline and 0.3 g of 3-formyl-1H-indole-5-carbonitrile was added to a clean and dry round bottom flask. 100 mL of 200 proof ethanol was added to flask. Solution was heated to reflux for 48 hrs. Extraction glassware with cellulose thimble filled with molecular sieves was used for reflux. After 48 hrs. of reflux, solution was cooled to room temperature and stored in the freezer for 18 hrs. Vacuum filtration was performed to collect 0.7 g (68.4%) of pale yellow colored fluffy solid. Molecular formula: C₃₆H₃₂N₆O₂. ESI(+) MS: m/z [M+H⁺] calculated for compound C₃₆H₃₂N₆O₂ 581.27, found 581.26.

### GK580.HCl Synthesis

60 mg of GK580 was transferred to a clean 7 mL vial. 1 mL of 1M HCl was added to the vial and set to mixing using a digital vortex mixer for 2 hrs. After 2 hrs. of mixing, solution was transferred to a round bottom flask and concentrated at 80° C. to furnish bright yellow colored powder. The compound was analyzed on mass spec to confirm the product. ESI(+) MS: m/z [M] calculated for the compound C₃₆H₃₃N₆O₂ 581.27, found 581.27. The reaction scheme is shown in FIG. 71.

GK578 Synthesis (4-bromo-2-((E)-((4-bromo-2-((Z)-2-(7-methoxy-4,9-dihydro-3H-pyrido[3,4-b]indol-1-yl)vinyl)phenyl)imino)methyl)aniline)

In this reaction, Harmaline and Aldehyde 1:1 adducts are synthesized by adding DI water to the reaction flask which inhibits formation of harmaline dimers. No Harmaline dimer was detected when DI water was used in the reaction flask. However, the amine of the aldehyde still reacted with the second aldehyde (see mechanism) to form this new polycyclic structure. In the reaction, 0.18 g of Harmaline and 0.1 g of 2-Amino-5-bromobenzaldehyde was added to a clean round bottom flask. 100 mL of 200-proof ethanol was added to the flask followed by 50 uL of DI water. Flask was equipped with condenser and setup was heated to 80-90° C. for 48 hrs under nitrogen. After 48 hrs. of reflux, solution was cooled to room temperature and stored in the freezer for 18 hrs. Vacuum filtration was performed to collect 0.1 g (34.5%) of bright yellow colored solid. Molecular formula: C₂₇H₂₂Br₂N₄O. ESI(+) MS: m/z [M+H⁺] calculated for the compound C₂₇H₂₂Br₂N₄O 579.02, found 579.03 (exact mass 576.02, molecular weight 578.31). The overall reaction scheme is shown in FIG. 72A, with the expected reaction mechanism shown in FIG. 72B. The ESI(+)MS molecular peak pattern (6 peaks) indicates the presence of 2-bromines in the structure.

### Example 10

Here, we show the MIC graphs for compounds GK580 and its HCl salt, GK580.HCl against S. aureus, MRSA, E. faecalis and VREF. GK580 and GK580.HCl show bacterial growth inhibition at 5.5 and 7 μg/mL (FIG. 73A) and kills bacteria at 11 and 7 μg/mL against S. aureus, respectively. Against MRSA, GK580 and GK580.HCl show bacterial growth inhibition at 3.5 and 7 μg/mL (FIG. 73B) and kills bacteria at 7 and 14 μg/mL, respectively. We tested GK580 and GK580.HCl against nine different MRSA strains—BAA-1763, 1766, 1717, 1747, 1754, 42, 1683, 2094 and 33592. MIC range is from 2.75 to 11 μg/mL for both GK580 and GK580.HCl. Against E. faecalis, GK580 and GK580.HCl show bacterial growth inhibition at 44 and 11 μg/mL (FIG. 73C) and kills bacteria at >88 and 28 μg/mL. GK580.HCl show bacterial growth inhibition at 14 μg/mL and kills bacteria at 44 μg/mL against VREF (FIG. 73D). No antibacterial activity was observed up 88 μg/ml for GK580 against VREF. We also tested vancomycin HCl salt against S. aureus, MRSA, E. faecalis and VREF. Vancomycin shows bacterial growth inhibition and kill at 1.56 μg/mL against S. aureus and MRSA, however it shows inhibition at 3.12 and 25 μg/mL and kills bacteria at 6.25 and >100 μg/mL against E. faecalis and VREF, respectively.

We performed UMU-ChromoTest assay to assess genotoxicity of GK580, GK580.HCl and GK660 (FIG. 74). GK580 does not show genotoxicity up to 30 ug/mL, however GK580.HCl show no genotoxicity upto testing concentration, 88 μg/mL. GK660 show no genotoxicity up to 80 μg/mL.

GK578 and salt also showed 50% inhibition in growth when incubated with E. coli for 10-75 hrs (FIG. 75). GK578 is precursor to competitive PABA inhibitor which showed significant reduction in growth of E. coli at 3.12 ug/mL. The same inhibition behavior was also exhibited by GK578.HCl.

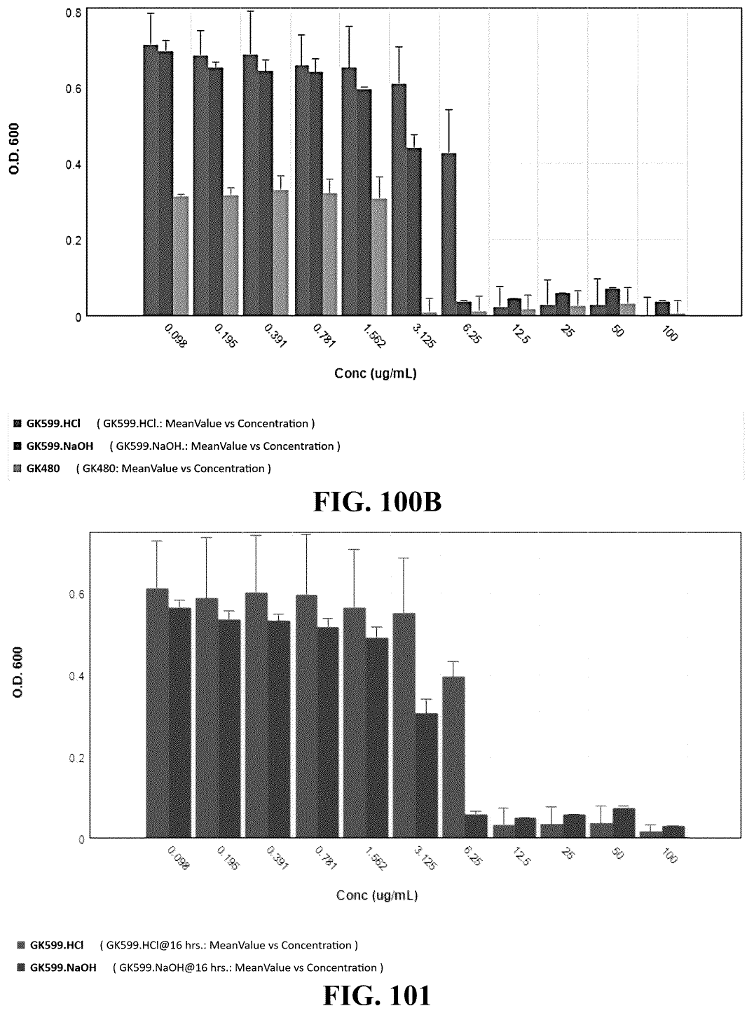

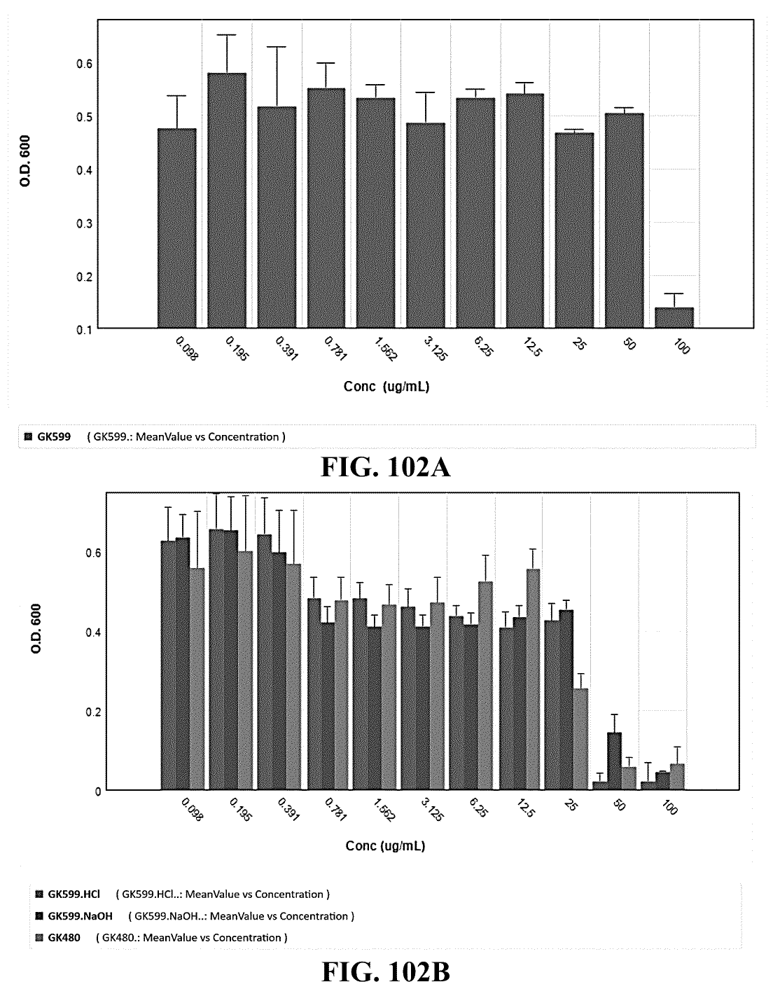

GK599 shows activity on S. aureus at 6.25 ug/mL (FIG. 100A), where as its HCl (GK599.HCl) and NaOH (GK599.NaOH) salts show activity at 12.5 ug/mL and 6.25 ug/mL respectively (FIG. 100B). On MRSA, GK599.NaOH shows activity at 6.25 ug/mL and GK599.HCl shows activity at 12.5 ug/mL (FIG. 101). On E. coli, both GK599 and GK599.NaOH show activity at 100 ug/mL, while GK599.HCl shows activity at 50 ug/mL (FIGS. 102A and 102B). GK480 also shows activity on S. aureus at 3.25 ug/mL and 50 ug/mL on E. coli (FIGS. 100B and 102B)

### Example 11

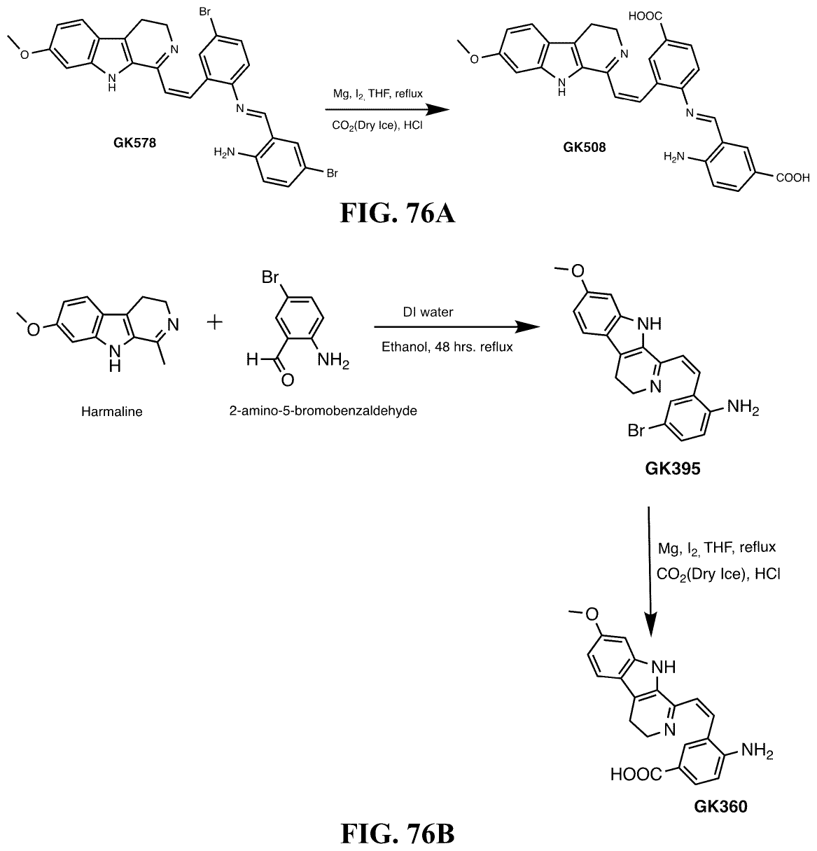

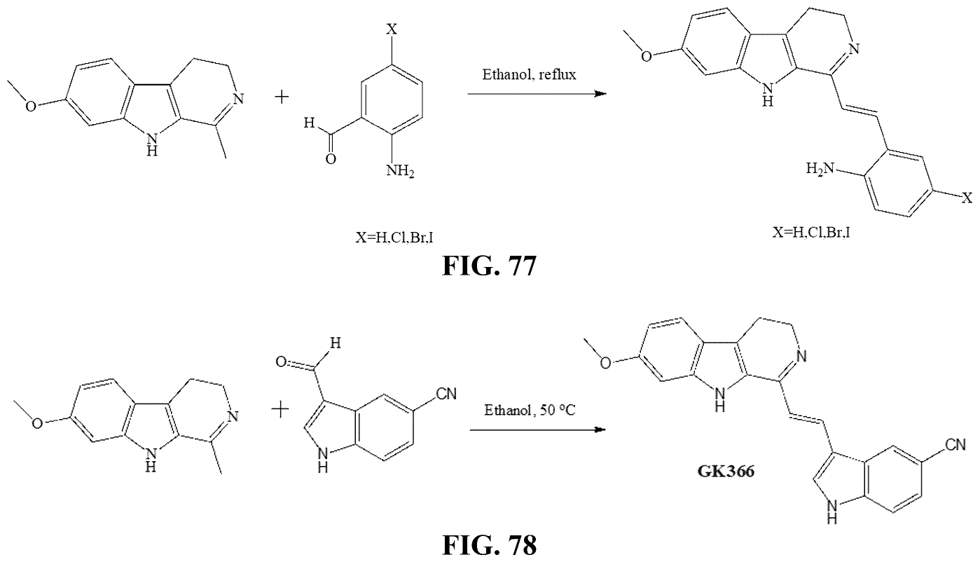

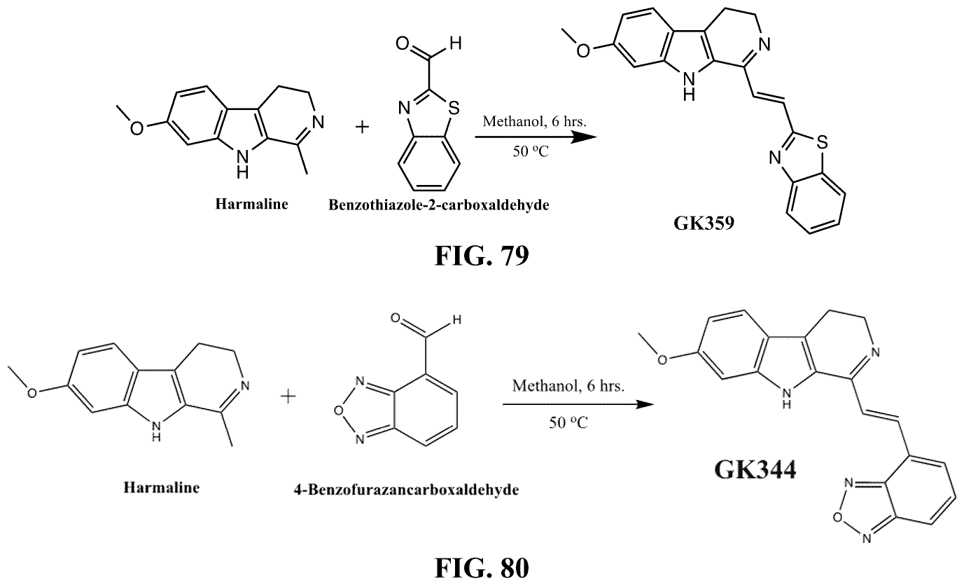

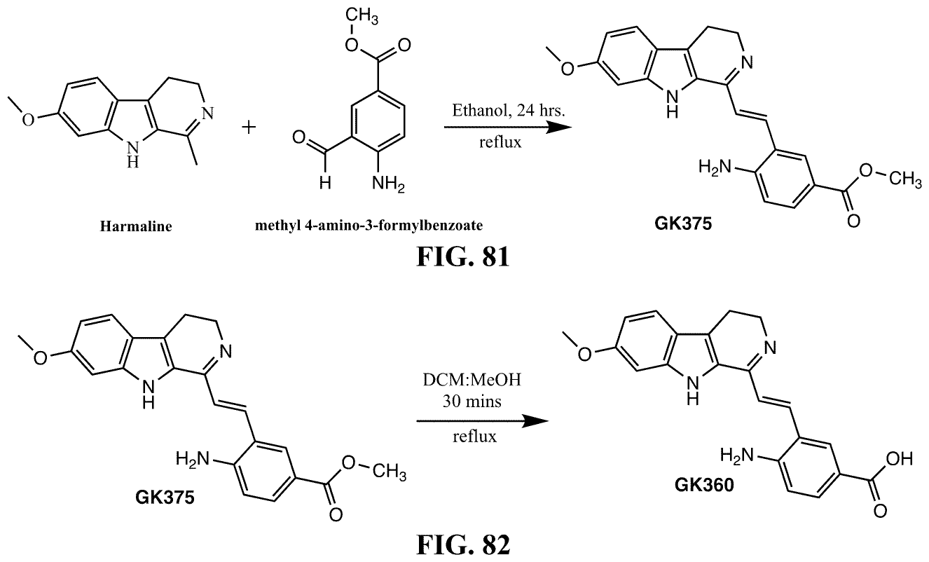

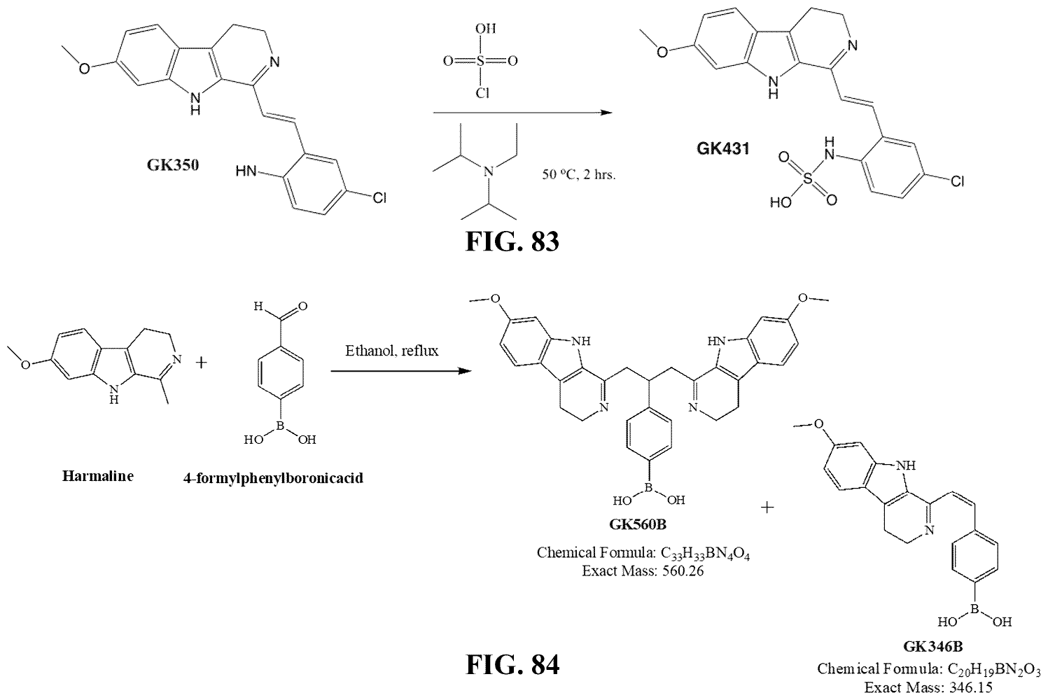

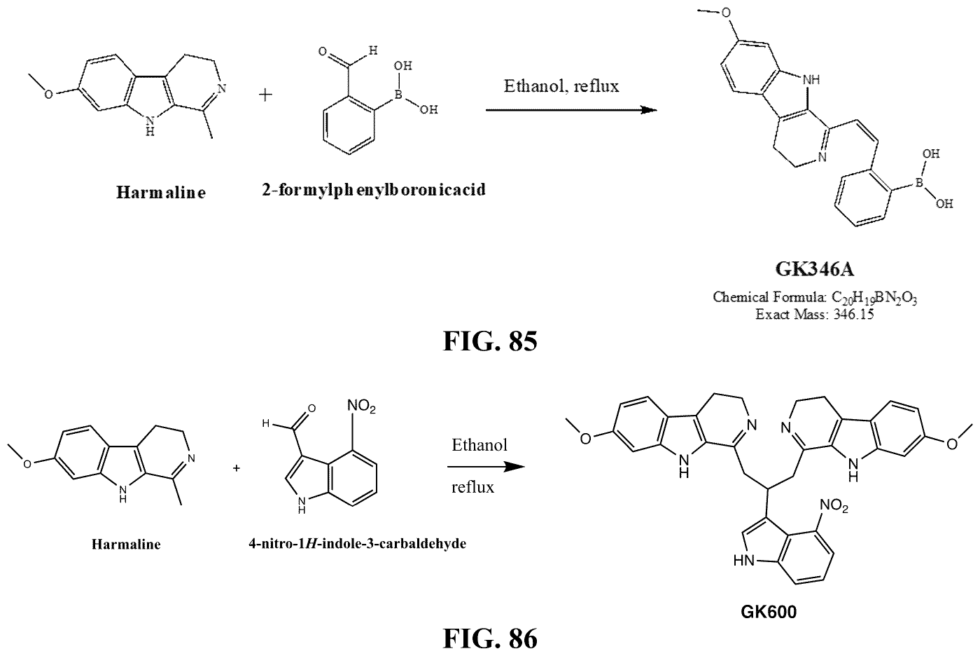

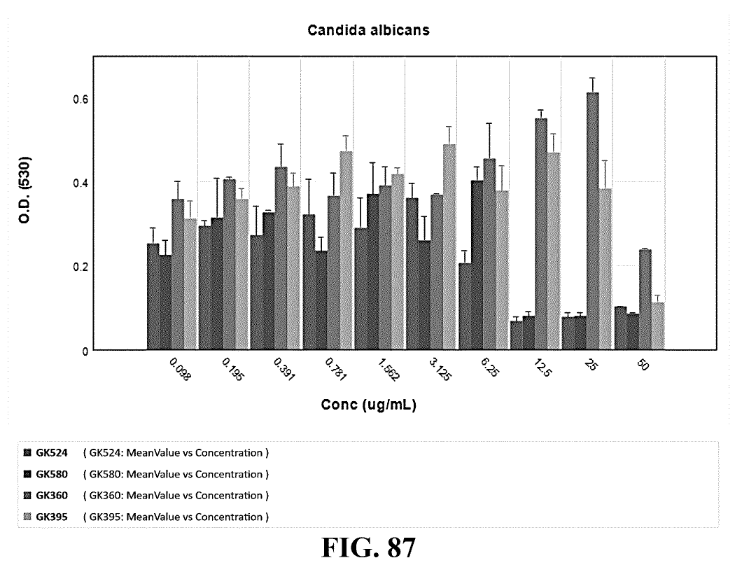

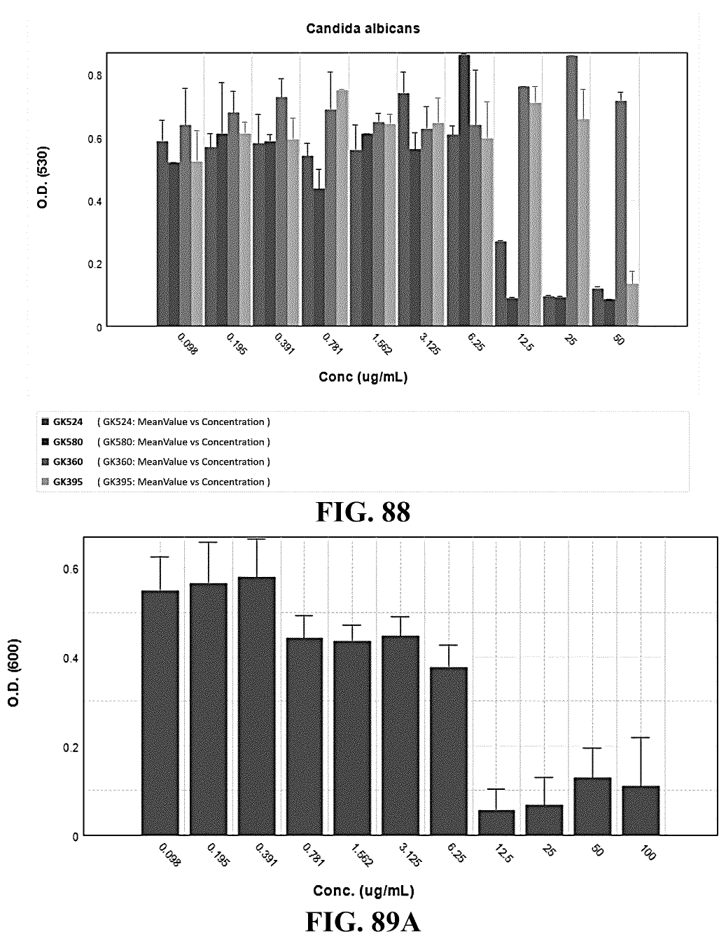

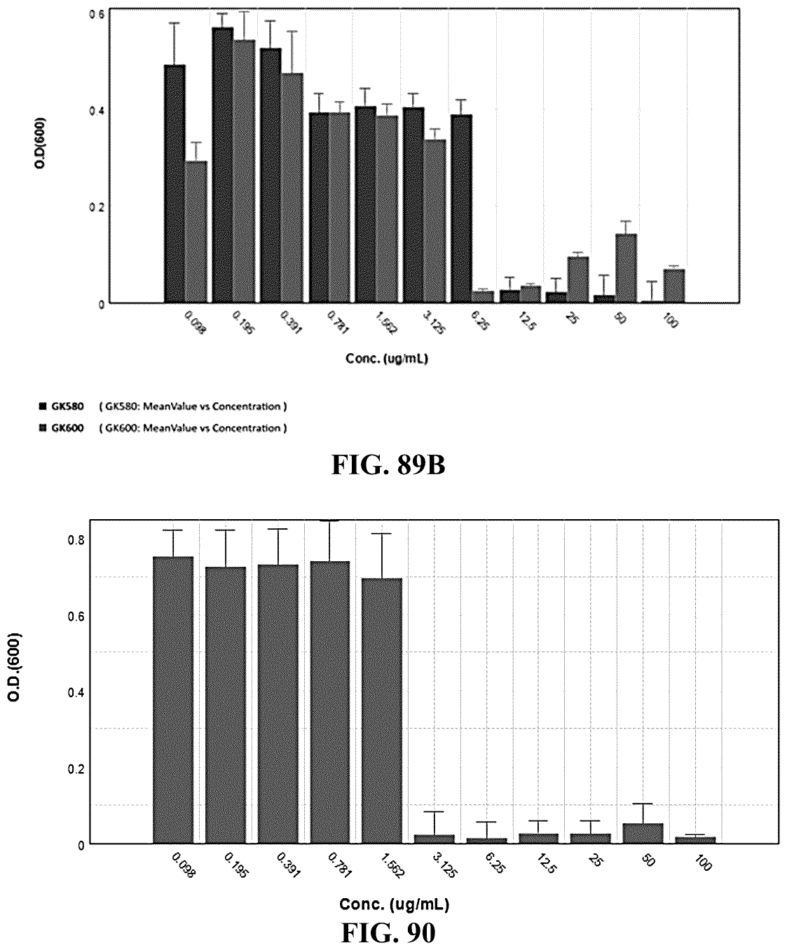

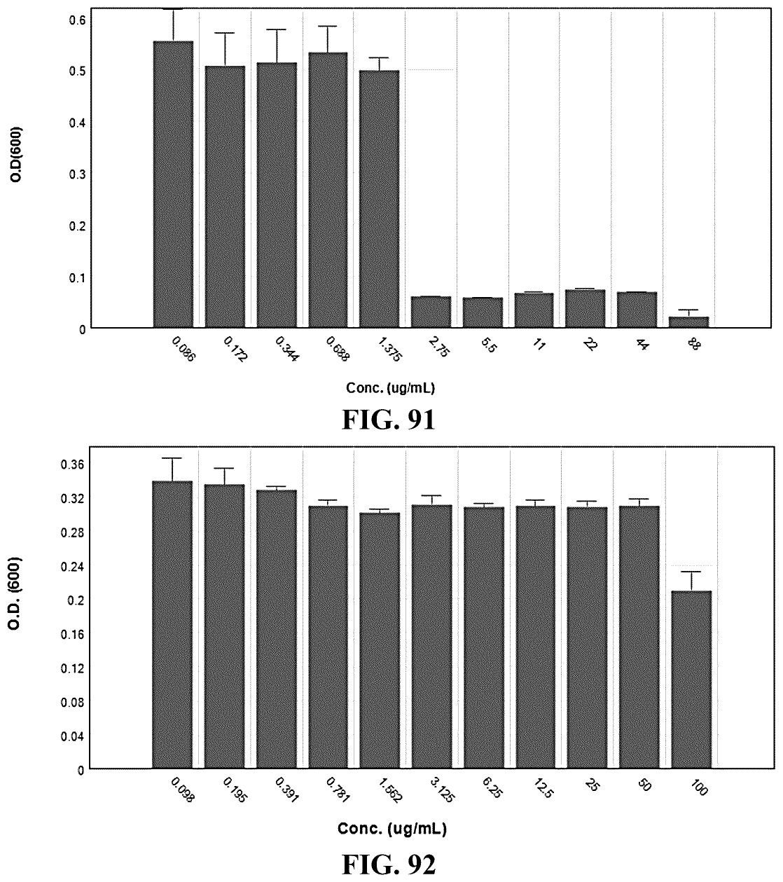

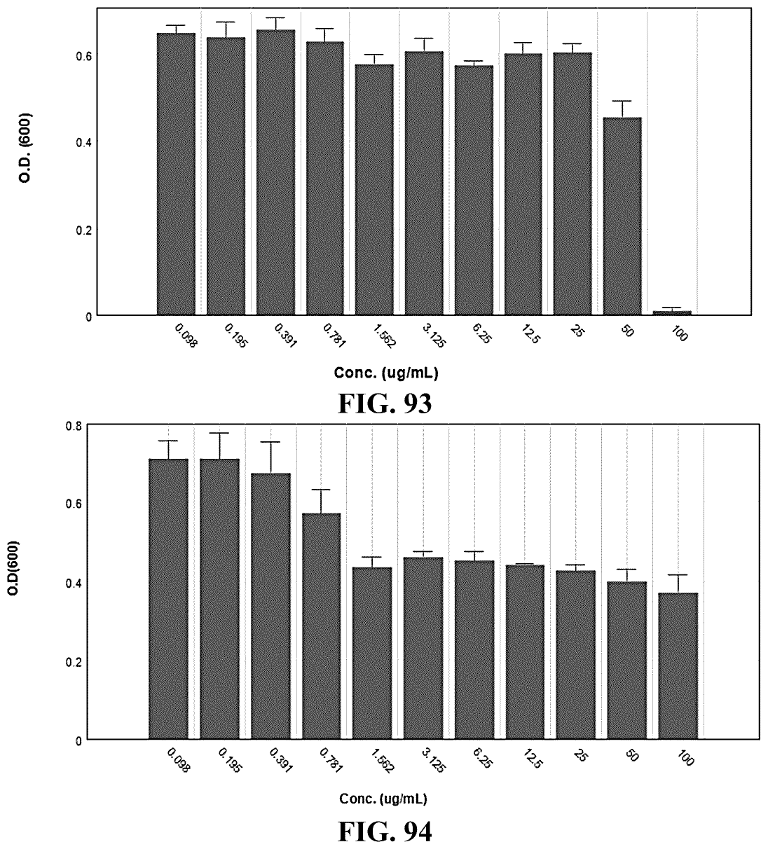

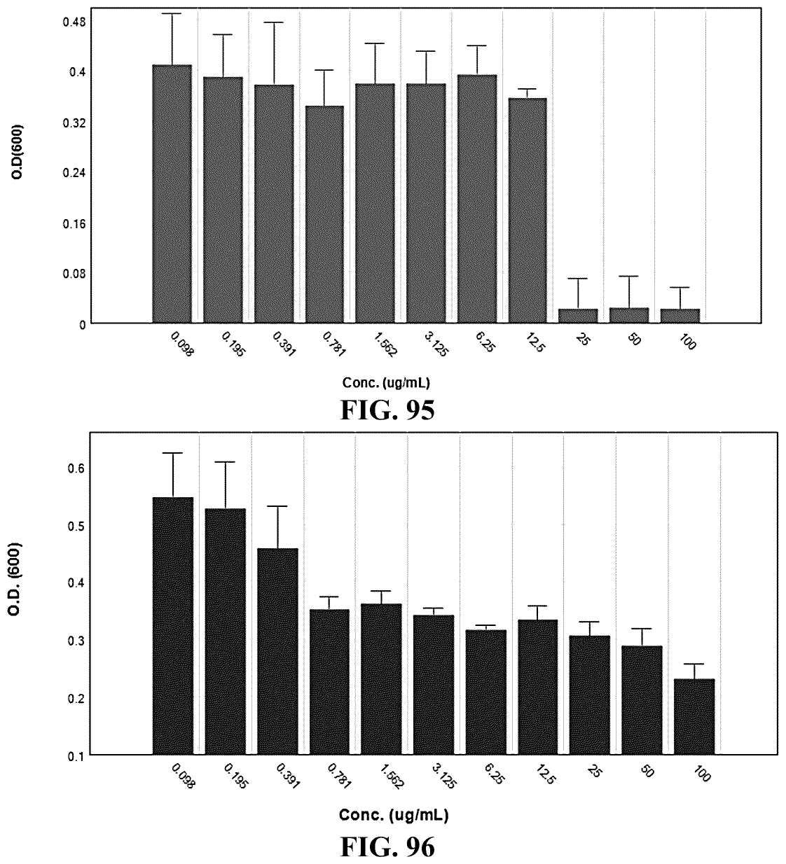

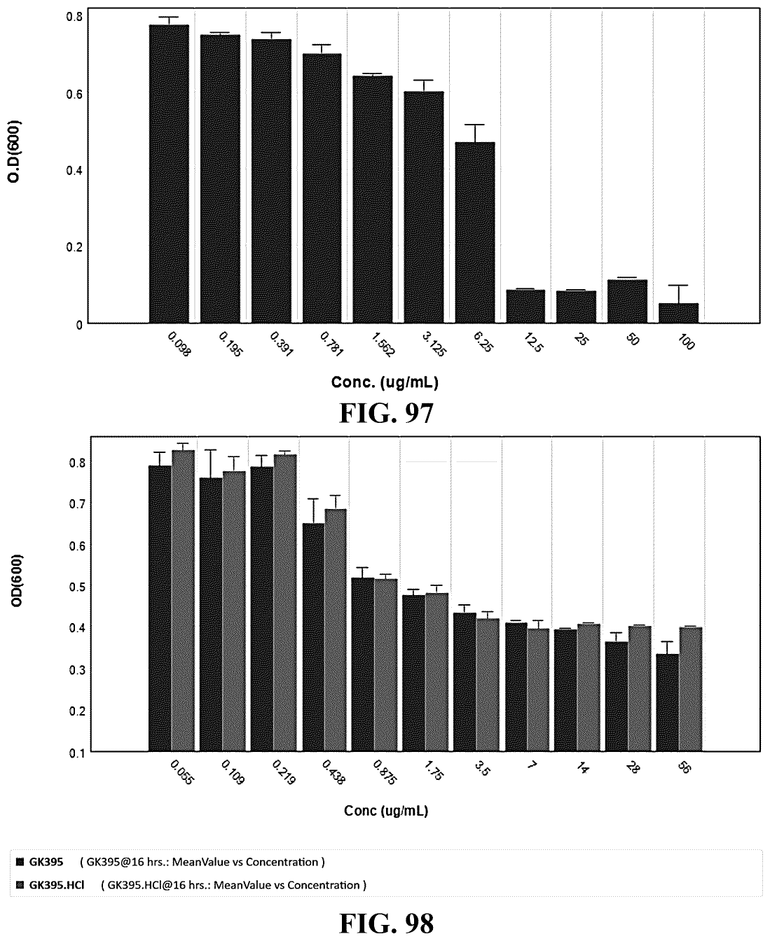

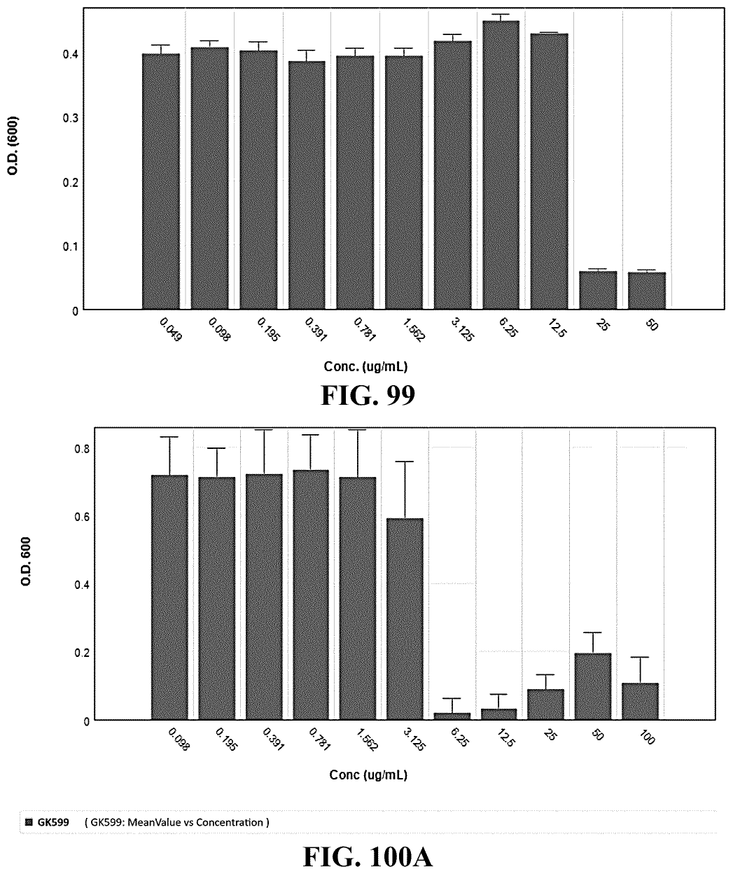

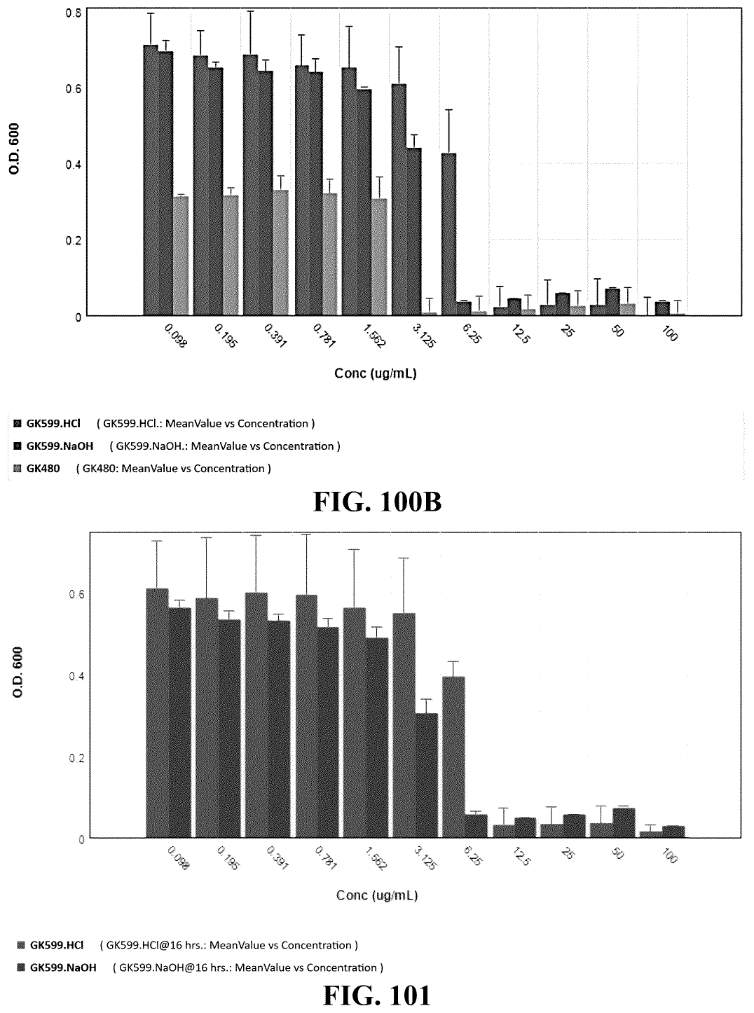

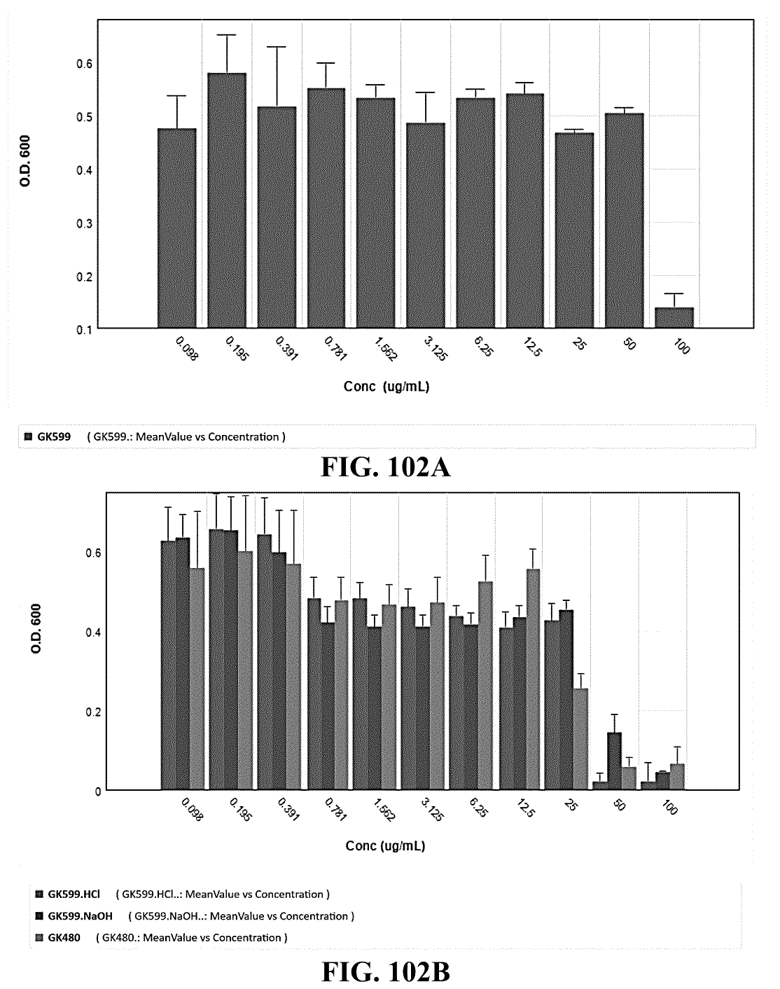

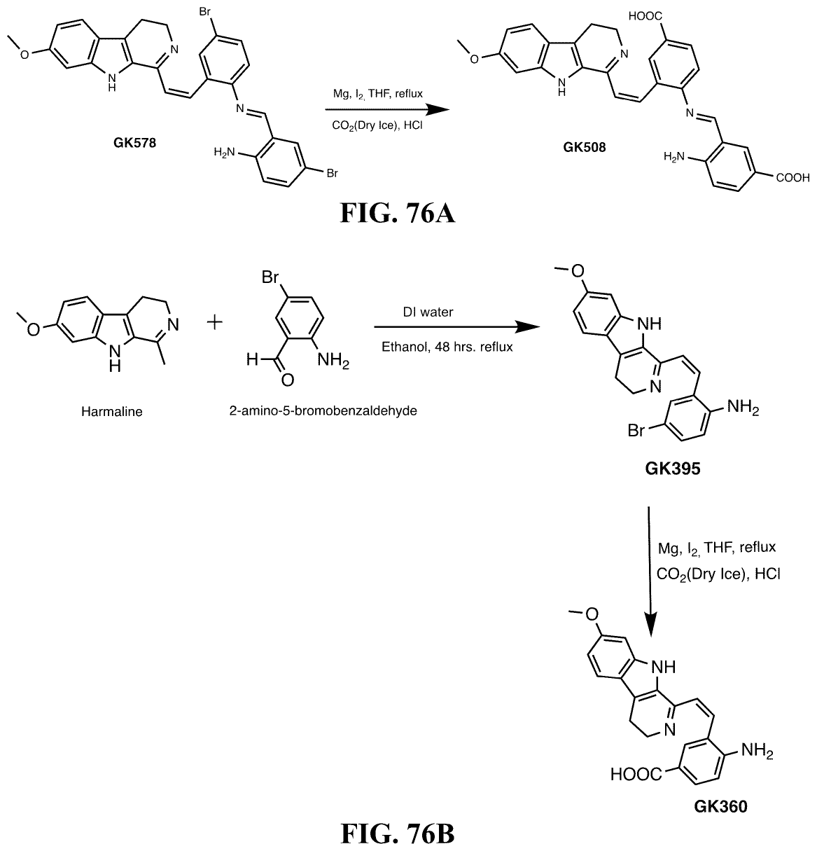

GK578 and GK395 can also be used as precursors to synthesize molecules designed to inhibit gram negative bacteria, especially E. coli, as shown in the reaction schemes in FIGS. 76A and 76B. The following general structures for competitive PABA inhibitors with expected activity on gram negative bacteria, especially E. coli, are as follows:

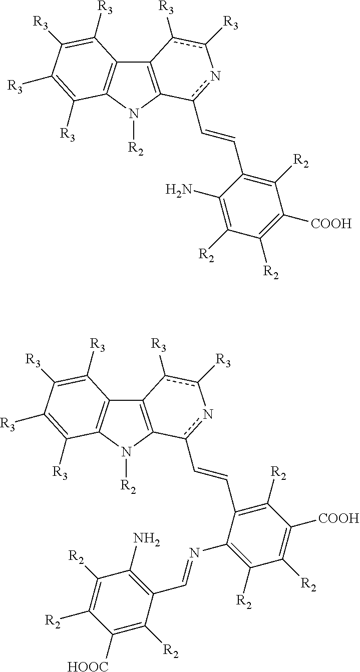

Possible Groups for R2 and R3: H, halogens, Sulfur, Thiols, alkyls(C1-C15), Nitro group, hydroxy, alkoxy (C1-C15), H-bond donor groups (e.g., carboxylic acids, amine and amides), H-bond acceptor groups (e.g., Ethers, Esters, Aldehydes, ketones), Aromatic rings with sizes from 4-10 carbons, Heterocyclic rings with size 4-10 atoms, Aromatic or heterocycle rings with substitutions like halogens, alkyls(C1-C15), hydroxy, H-bond donor groups, or H-bond acceptor groups. Fused heterocyclic rings (Thioridazine, purines, coumarins, Indoles, Indazole) with substitutions at one or more carbons of the ring.

### Example 12

### 1. Synthesis of GK317, GK395, GK350, GK443

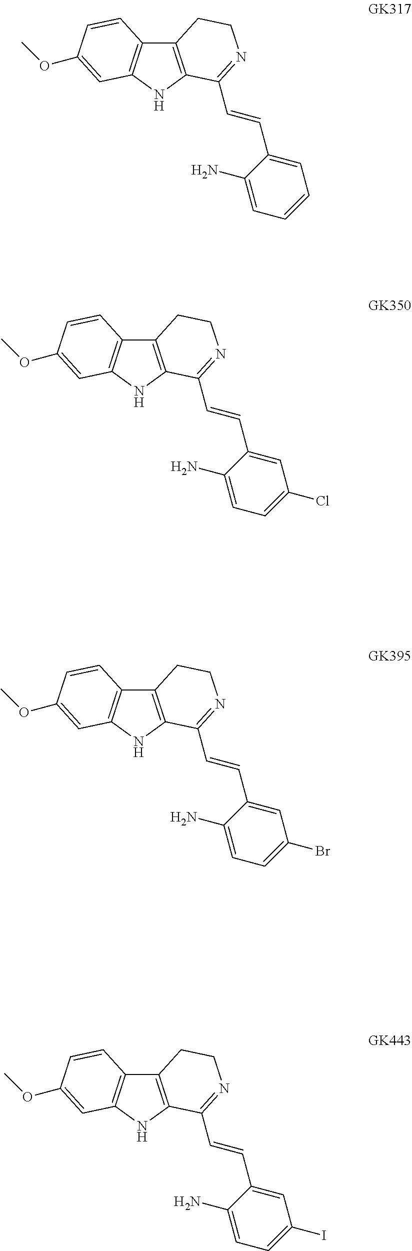 [SMILES: COc1ccc2c3c([nH]c2c1)C(/C=C/c1ccccc1N)=NCC3 ; COc1ccc2c3c([nH]c2c1)C(/C=C/c1cc(Cl)ccc1N)=NCC3 ; COc1ccc2c3c([nH]c2c1)C(/C=C/c1cc(Br)ccc1N)=NCC3 ; COc1ccc2c3c([nH]c2c1)C(/C=C/c1cc(I)ccc1N)=NCC3]

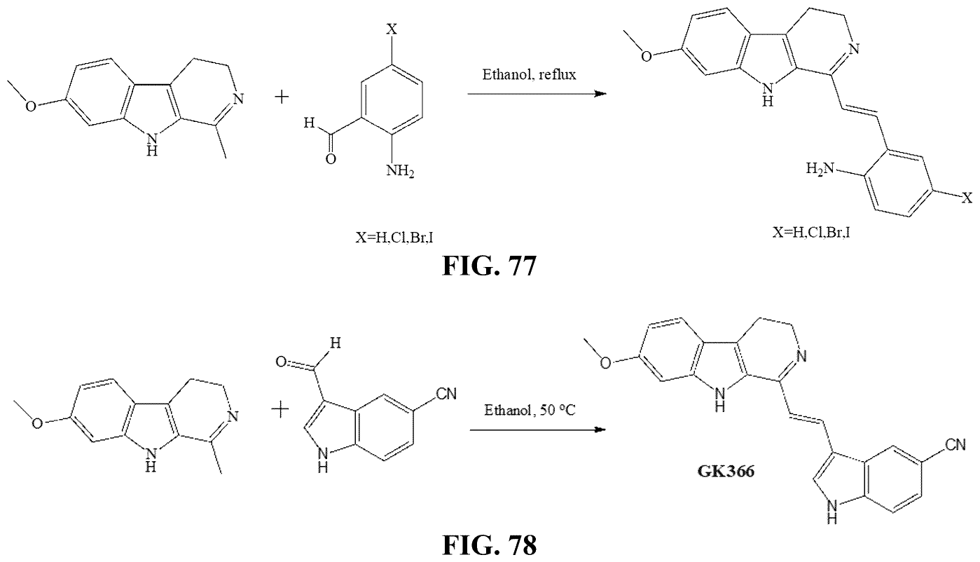

The general reaction scheme is shown in FIG. 77. GK317, GK350, GK395 are inspired from β-aminobenzoic acid (PABA), which is essential for bacterial cell wall synthesis. We tried to resemble PABA structure to make a potential competitive inhibitor. GK317 has no halogen, GK350 has chlorine, GK395 has bromine and GK360 (below) has carboxylic acid.

General Procedure: 1 eq of Aldehyde was added to a clean flask with 0.8-1 eq of Harmaline. 100 mL of 200 proof ethanol was added to flask and setup for 24 hrs. reflux. After 24 hrs., solution was cooled to room temperature, further cooling was achieved by keeping flask in freezer for 18 hrs. Vacuum filtration was performed to collect the solid.

GK317: 100 mg of 2-aminobenzaldehyde and 140 mg of Harmaline react together to furnish 85 mg (40.6%) of GK317. No dimer formation was observed. Formation of polymer products at R.T 3.16 and 5.10 observed. The polymer products were turned into desired compound by reacting with 0.1 N HCl. ESI(+)MS: m/z [M+H⁺] calculated for C₂₀H₁₉N₃O 318.16; found 318.16.

GK350: 200 mg of 2-amino-5-chlorobenzaldehyde and 250 mg of Harmaline react together to furnish 115 mg (28.3%) of GK350. No dimer formation was observed. ESI(+)MS: m/z [M+H⁺] calculated for C₂₀H₁₈ClN₃O 352.12; found 352.12.

GK395: 400 mg of 2-amino-5-bromobenzaldehyde and 400 mg of Harmaline react together to furnish 250 mg (30.7%) of GK395. ESI(+)MS: m/z [M+H⁺] calculated for C₂₀H₁₈BrN₃O 396.07; found 396.08.

GK443: This reaction was unsuccessful because 2-amino-5-iodobenzaldehyde was not soluble in the solvent. No reaction took place, and the compound could not be synthesized. 2. Synthesis of GK366

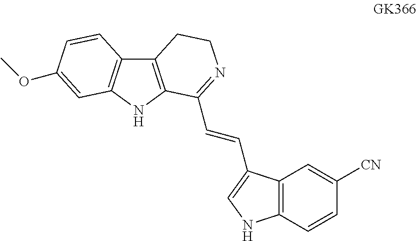 [SMILES: COc1ccc2c3c([nH]c2c1)C(/C=C/c1c[nH]c2ccc(C#N)cc12)=NCC3]

The general reaction scheme is shown in FIG. 78. 420 mg of 3-formyl-1H-indole-5-carbonitrile was added to a clean 250 mL flask followed by 610 mg of Harmaline. 100 mL of 200 proof ethanol was added to the flask and condenser was attached. 1 mL of DI water was added to the flask and the flask was heated to 50° C. for 6 hrs. Solution was cooled to room temperature and concentrated on rotavapor. 1 mg of crude solid was used to prepare a sample in Acetonitrile and Water mixture for LCMS analysis. ESI(+)MS: m/z [M+H⁺] calculated for C₂₃H₁₈N₄O 367.16; found 367.15. yield was 20.82%

### 3. Synthesis of GK359

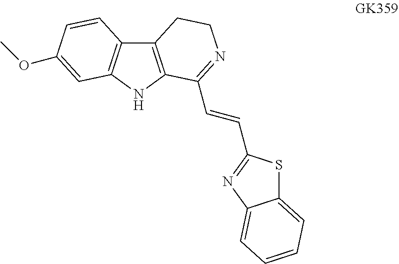 [SMILES: COc1ccc2c3c([nH]c2c1)C(/C=C/c1nc2ccccc2s1)=NCC3]

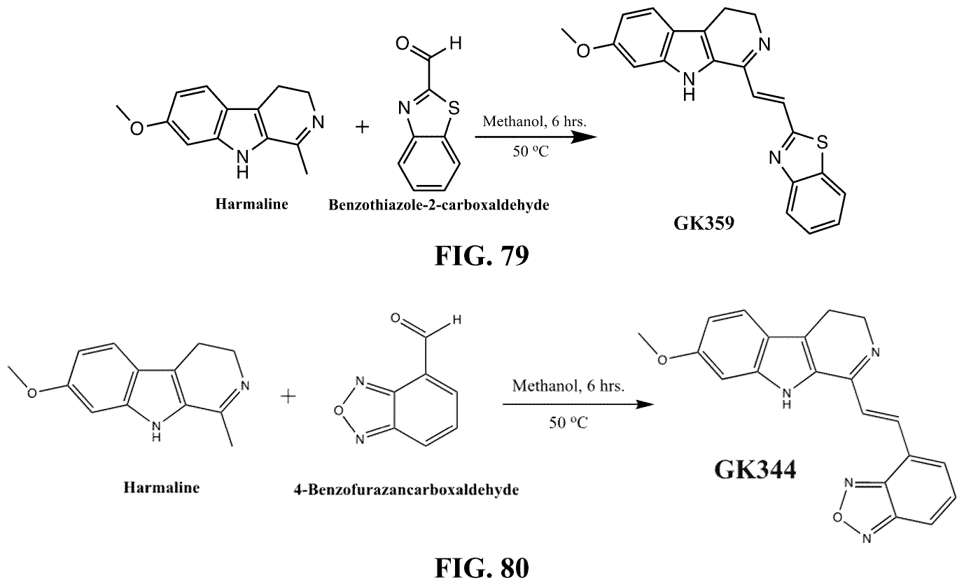

The general reaction scheme is shown in FIG. 79. 330 mg of Harmaline was added to a vial with 300 mg of Benzothiazole-2-carboxaldehyde and 250 μL of DI water. 35 mL of LCMS grade methanol was added to vial and the solution was mixed well using digital vortex mixer. The vial was placed in a heat bath at 50° C. After 6 hrs., 10 μL of the solution was used to dilute to 1 mL with LCMC grade methanol. 100 uL of the above solution was diluted to 1 mL using Acetonitrile and water mixture for LCMS analysis. ESI(+)MS: m/z [M+H⁺] calculated for C₂₁H₁₇N₃OS 360.12; found 360.11. yield was 19.55%.

### 4. Attempted Synthesis of GK344

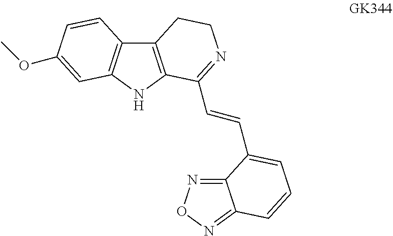 [SMILES: COc1ccc2c3c([nH]c2c1)C(/C=C/c1cccc2nonc12)=NCC3]

The general reaction scheme is shown in FIG. 80. 368 mg of Harmaline was added to a vial with 300 mg of 4-Benzofurazancarboxaldehyde and 250 μL of DI water. 35 mL of LCMS grade methanol was added to vial and the solution was mixed using digital vortex mixer. Vial was placed in a heat bath at 50° C. After 6 hrs., 10 μL of the solution was used to dilute to 1 mL with LCMC grade methanol. 100 uL of the above solution was diluted to 1 mL using Acetonitrile and water mixture for LCMS analysis. ESI(+)MS: m/z [M+H⁺] calculated for C₂₀H₁₆N₄O₂ 345.13; found 345.13. yield was 28.97%.

### 5. Synthesis of GK375

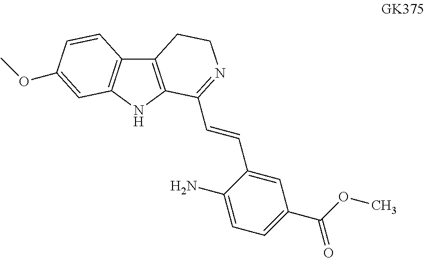

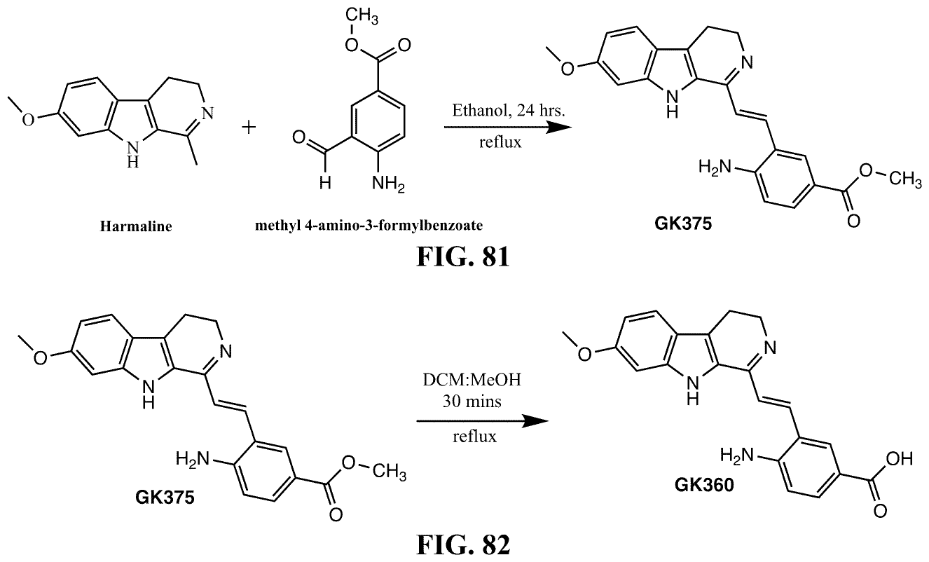

The general reaction scheme is shown in FIG. 81. 100 mg of 4-Amino-3-formyl-benzoic acid methyl ester was transferred to a clean 250 mL reaction flask. 150 mL of 200 proof ethanol was added to flask followed by 2 mL of DI water. 110 mg of Harmaline was added to the reaction flask and setup for 24 hrs. reflux. After 24 hrs., the flask was cooled to room temperature and further cooling was achieved by keeping it in freezer for 18 hrs. Compound precipitation was observed in the flask. Vacuum filtration was performed to collect the solids and cold ethanol was used to wash the solid on filter paper to remove traces of impurities. Solid was left on vacuum for 15 more minutes to dry. 145 mg (73.9% yield) of solid was transferred to a clean vial and stored in freezer. 1 mg of the solid was diluted in 1 mL of methanol. 100 uL of the solution was diluted to 1 mL using Acetonitrile and water mixture for LCMS analysis. ESI(+)MS: m/z [M+H⁺] calculated for C₂₂H₂₁N₃O₃ 376.17; found 376.17. yield was 28.97%.

### 6. Synthesis of GK360

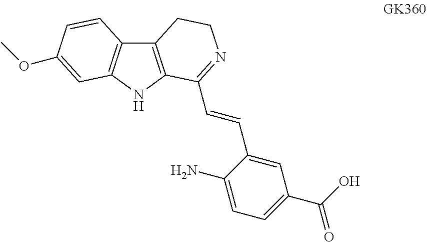 [SMILES: COc1ccc2c3c([nH]c2c1)C(/C=C/c1cc(C(=O)O)ccc1N)=NCC3]

The general reaction scheme is shown in FIG. 82. 30 mg of GK375 was dissolved in 9:1 mixture of CH₂Cl₂ and Methanol in a flask. 1 mL of 0.1 N NaOH was added and setup for reflux for 30 mins. Solution was concentrated on rotavapor, and a sample was prepared for LCMS analysis. LCMS data confirms the 100% conversion of GK375 into GK360. 361.14 found 361.19. ESI(+)MS: m/z [M+H⁺] calculated for C₂₂H₂₁N₃O₃ 376.17; found 376.17. yield was 100%. Hydrolysis of ester can also be achieved by Acid hydrolysis.

### 7. Synthesis of GK431

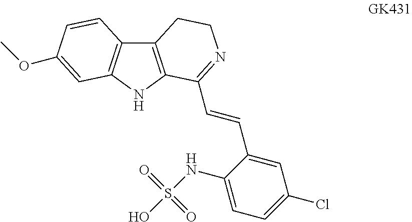 [SMILES: COc1ccc2c3c([nH]c2c1)C(/C=C/c1cc(Cl)ccc1NS(=O)(=O)O)=NCC3]

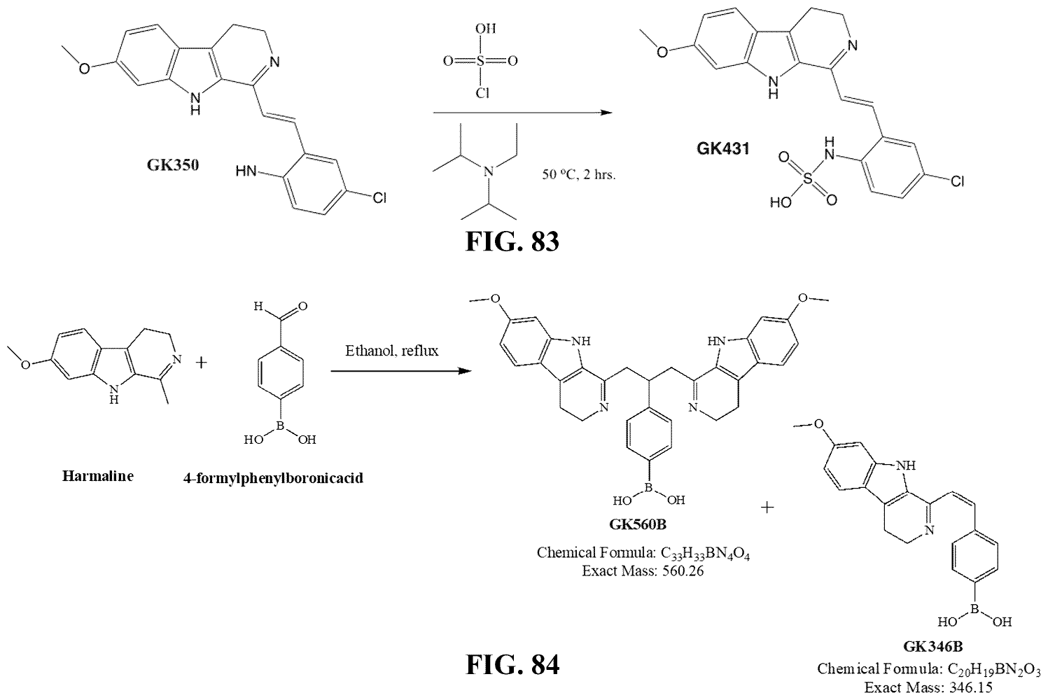

The general reaction scheme is shown in FIG. 83. 50 mg of pure GK350 was added to a 4 mL vial followed by 16 uL of chlorosulfonic acid and 18 uL of N-Ethyldiisopropylamine. The vial was placed in heat bath at 50° C. for 3 hrs. After 3 hrs, solution was concentrated and a sample was prepared for LCMS analysis. ESI(+)MS: m/z [M+H⁺] calculated for C₂₀H₁₈ClN₃O_{4S} 432.08; found 432.07. yield was 14.5%.

### 8. Synthesis of GK560B,GK346B

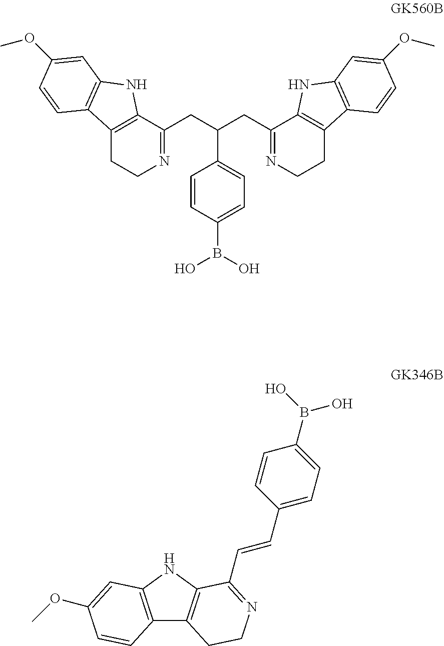

The general reaction scheme is shown in FIG. 84. 300 mg of 4-formylphenylboronicacid was added to a clean flask equipped with a magnetic stir bar. 1130 mg of Harmaline was added to the flask followed by 150 mL of 200 proof Ethanol. Solution was set to reflux for 24 hrs. After the reaction time, solution was cooled to room temperature and flask was stored in freezer for 18 hrs. No precipitation was observed. The solution was concentrated on rotavapor to furnish 1350 mg of brown color solid. 1 mg of the crude was used to dilute in Acetonitrile and water mixture for LCMS analysis. From the data, Harmaline, was 37.78%, GK560B 48.70% and Adduct GK346B was 13.52% present in the crude. ESI(+)MS: m/z [M+H⁺] calculated for C₃₃H₃₃BN₄O₄ 561.27; found 561.27. m/z [M+H⁺] calculated for C₂₀H₂₀BN₂O₃ 347.16; found 347.15.

### 9. Synthesis of GK346A

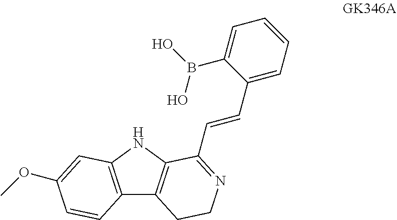 [SMILES: COc1ccc2c3c([nH]c2c1)C(/C=C/c1ccccc1B(O)O)=NCC3]

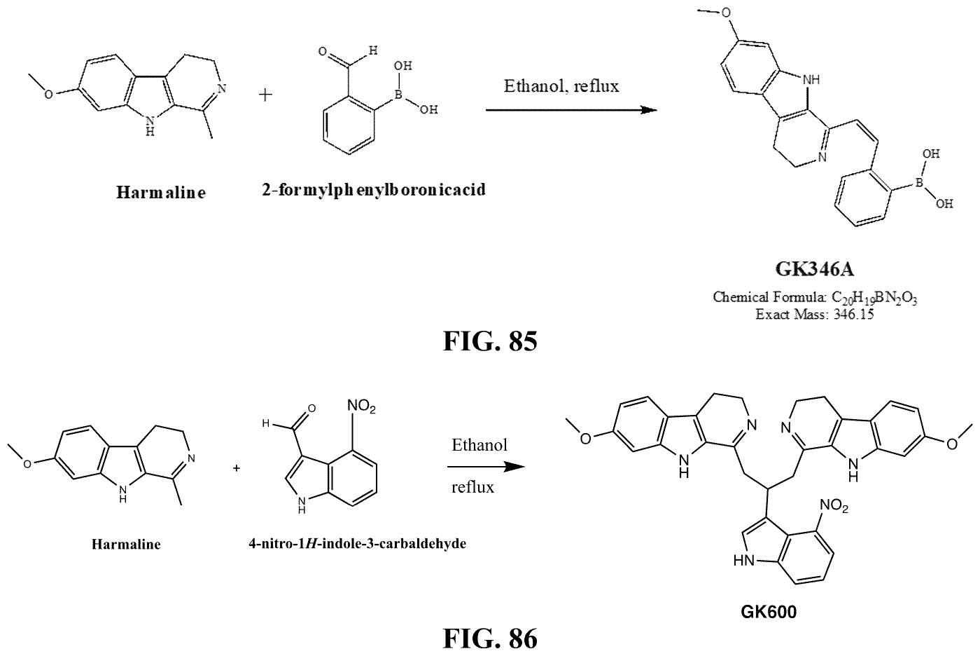

The general reaction scheme is shown in FIG. 85. 200 mg of 2-formylphenylboronicacid was added to a clean flask equipped with a magnetic stir bar. 750 mg of Harmaline was added to the flask followed by 150 mL of 200 proof Ethanol. Solution was set to reflux for 24 hrs. After the reaction time, solution was cooled to room temperature and flask was stored in freezer for 18 hrs. No precipitation was observed. The solution was concentrated on rotavapor to furnish brown color solid. 1 mg of the crude was used to dilute in Acetonitrile and water mixture for LCMS analysis. From the data, Harmaline, was 46.64%, GK346A, was 53.2% present in the crude. No dimer, GK560A was observed in LCMS analysis. m/z [M+H⁺] calculated for C₂₀H₂₀BN₂O₃ 347.16; found 347.15. 610. Synthesis of GK600

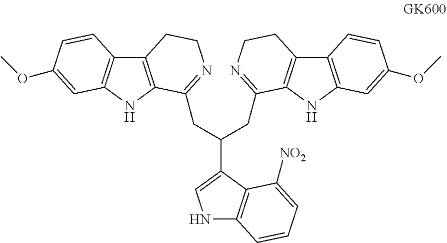

The general reaction scheme is shown in FIG. 86. 890 mg of Harmaline and 300 mg of 4-nitroindole-3-carboxaldehyde was added to a clean and dry round bottom flask. 150 mL of 200 proof ethanol was added to flask. Solution was heated to reflux for 48 hrs. After 48 hrs. of reflux, solution was cooled to Room Temperature and stored in freezer for 18 hrs. to improve crystallization of product. Vacuum filtration was performed to collect 800 mg (84%) of olive green colored solid. LCMS confirms the desired compound GK600. m/z [M+H⁺] calculated for C₃₅H₃₂N₆O₄ 601.26; found 601.25.

### 11. Observations:

In the reactions between Harmaline and Aldehydes, Aldehydes with electron rich group (Electron donating group) like —NH₂, —B(OH)₂ on the next carbon to aldehydes functional group only furnish adducts but not dimer. E.g.: 2-formylphenylboronicacid, 2-aminobenzaldehyde, 2-amino-5-halobenzaldehyde.

| | | |
|---|---|---|
| 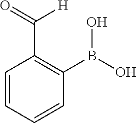 [SMILES: O=Cc1ccccc1B(O)O] | 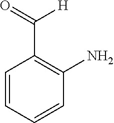 | Furnish only Harmaline, aldehyde 1:1 adducts but not Harmaline dimers |
| 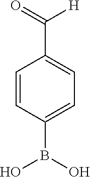 [SMILES: O=Cc1ccc(B(O)O)cc1] | 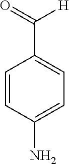 | Furnish both Harmaline, aldehyde 1:1 adducts and Harmaline dimers. |

### Example 13

### Antifungal Activity

### 1. Protocols

Synthesized compounds were tested for antifungal activity towards the fungus strain, Candida albicans. The concentration of compound required to visibly stop the growth of fungus is reported as minimum inhibitory concentration (MIC). Different concentrations of the compounds were tested for the exact same number of fungi, and the growth is monitored with time. MIC was determined by using the broth microdilution assay.

### Single Compound MIC Determination Via Broth Microdilution Assay

Fungus strain, C. albicans was purchased from ATCC (ID: 18804) for the studies. The MIC assay procedure follows the guidelines set by the CLSI and has the following steps:
(a) The procedure requires 96-well microdilution plates of untreated polystyrene with U-shaped wells for all studies.
(b) Preparation of Inoculum: C. albicans was subcultured from sterile vials onto Sabouraud dextrose agar and passaged to ensure purity and viability at 35° C. Approximately five colonies of ˜1 mm in diameter were picked and suspended in the sterile water or 0.145-mol/L saline (8.5 g/L NaCl; 0.85% saline), vortexed and adjusted to a transmittance that equals a 0.5 McFarland standard at a wavelength of 530 nm. This solution was used to make a working solution by preparing a 1:50 dilution, followed by a 1:20 dilution, with RPMI 1640 medium to obtain 2× test inoculum. The 2× inoculum is diluted 1:1 to achieve the final inoculum size (0.5×10³ to 2.5×10³ cells per mL). RPMI 1640 medium was used with L-glutamine and phenol red as a pH indicator but without bicarbonate and a glucose concentration of 0.2%. The RPMI 1640 medium was buffered to a pH of 7 with 3-(N-morpholino)propanesulfonic acid (MOPS) to produce consistent results and was used to develop the standard.
(c) Compound Dilution: All compounds used in the study were dissolved in sterile ultrapure DMSO (VWR International, PA, USA) and maintained at −80° C. Stock solution of the compound was freshly prepared the day of use or is prepared from a frozen stock solution stored at −80° C. The compound solution was prepared at desired concentration in DMSO, and 1/10 dilution was performed in sterile RPMI 1640 medium. This 10-fold solution of stock solution was used for a 2-fold serial dilution in a 96-well plate to observe an inhibitory effect of the compound on fungal growth. 100 μL of diluted compound solution was transferred to a first column of the 96-well plate. 50 μL of sterile RPMI 1640 medium was transferred to each well from well 2 to well 12. Next, 50 L was removed from well 1 and added to well 2. The contents from well 2 were mixed by pipetting up and down. Next, 50 μL were removed from well 2 and added to well 3. This process was continued till well 11 is reached. 50 μL was discarded from the well 11 and 50 μL RPMI 1640 medium was added to well 12. Each compound concentration was tested in triplicate.
(d) Inoculation of 96-well plate: 50 μL of working inoculum was carefully added to each well, except the sterility well 12. Mixing is performed by gently flushing the inoculum in and out of the pipette tip four or five times, avoiding splashing and bubble formation. Apart from the samples, three controls were used for the assay: (i) Growth control (GC), which have fungus but no tested compound to show how fungus grow without the compound and are incubated with diluted 2× inoculum suspensions; (ii) Sterility control (SC), which doesn't contain any fungus or compound and doesn't expected to show any fungal growth after incubation; and (iii) DMSO control (DC), which does not have compound to show the effect of DMSO on fungal growth.
(e) Incubation: Plates were incubated at 35° C. for 24 hours without shaking or agitation.
(f) Determining of MIC: After 24 hours of incubation, the growth control well was inspected for the presence or absence of growth. All plates were then read visually under normal laboratory lighting using a mirror viewer. Wells were scored for the growth compared to that of the drug free control well. Plates were read at 530 nm with subtraction of the blank from the reading of each well. Turbidity (OD) increases as the fungus grows over time. The lowest concentration at which there is a >90% decrease in growth is reported as MIC.

### 2. Antifungal Activity of Harmaline Dimer and Adduct Analogs

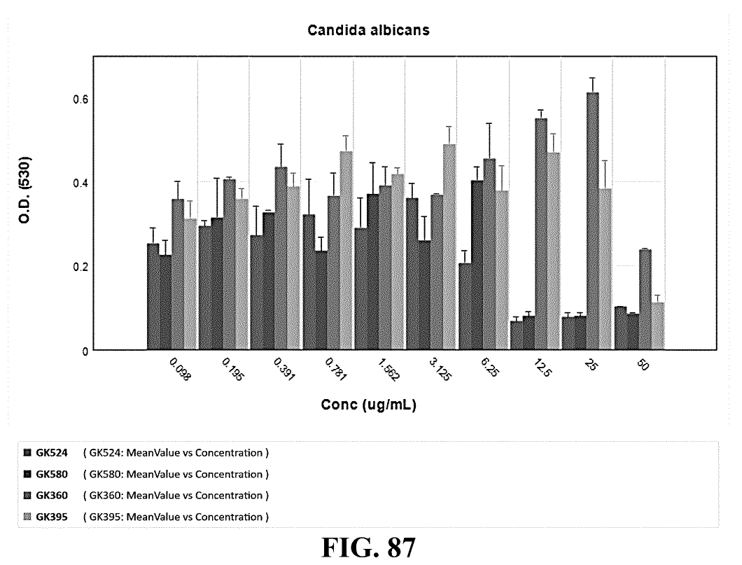

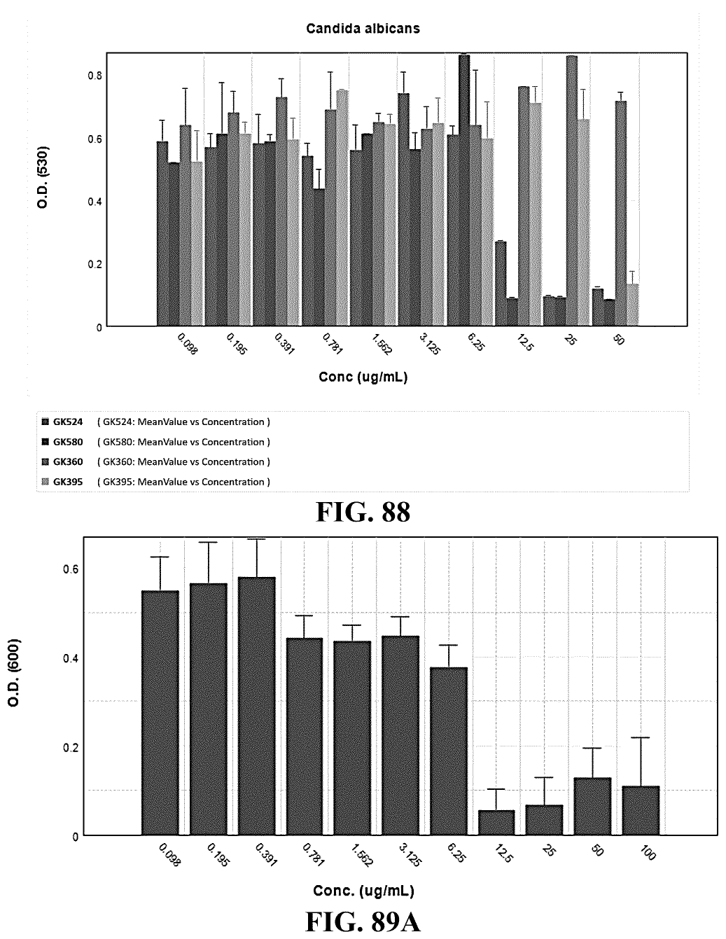

The antifungal activity of a set of novel harmaline dimers, GK580 and GK524 and adducts, GK360 and GK395 was evaluated. FIGS. 87 and 88 show the antifungal activity of harmaline dimers and adducts after 24 and 48 hours of incubation. In the antifungal activity screening, harmaline dimers, GK580 and GK524 showed antifungal growth inhibition at 12.25 μg/mL after 24 hours of incubation. After 48 hours of incubation, GK524 show increased MIC at 25 μg/mL. GK395 showed antifungal activity at 50 μg/mL after 48 hours of incubation. However, GK360 showed MIC at 50 μg/mL only after 24 hours of incubation.

Data suggest that the harmaline dimer molecules GK580 and GK524 show higher efficacy than the harmaline adducts GK395 and GK360. Specifically, GK580 with indole substitution at tether shows complete inhibition of antifungal growth at 12.25 μg/mL after 48 hours of incubation.

### Example 14

### Additional Antibacterial Data

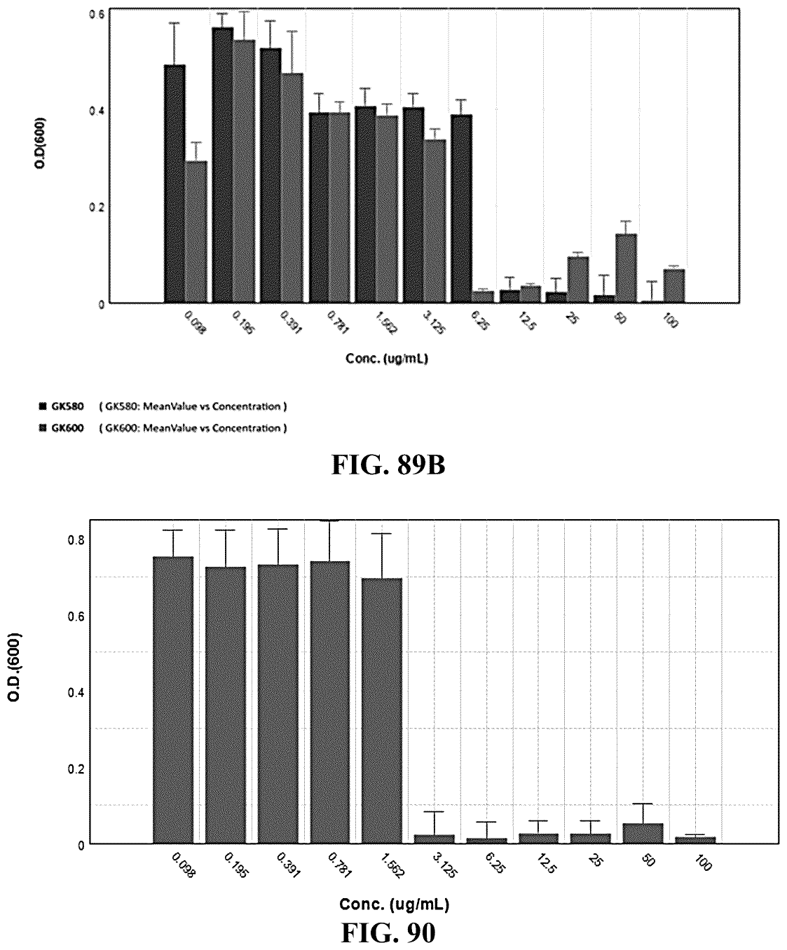

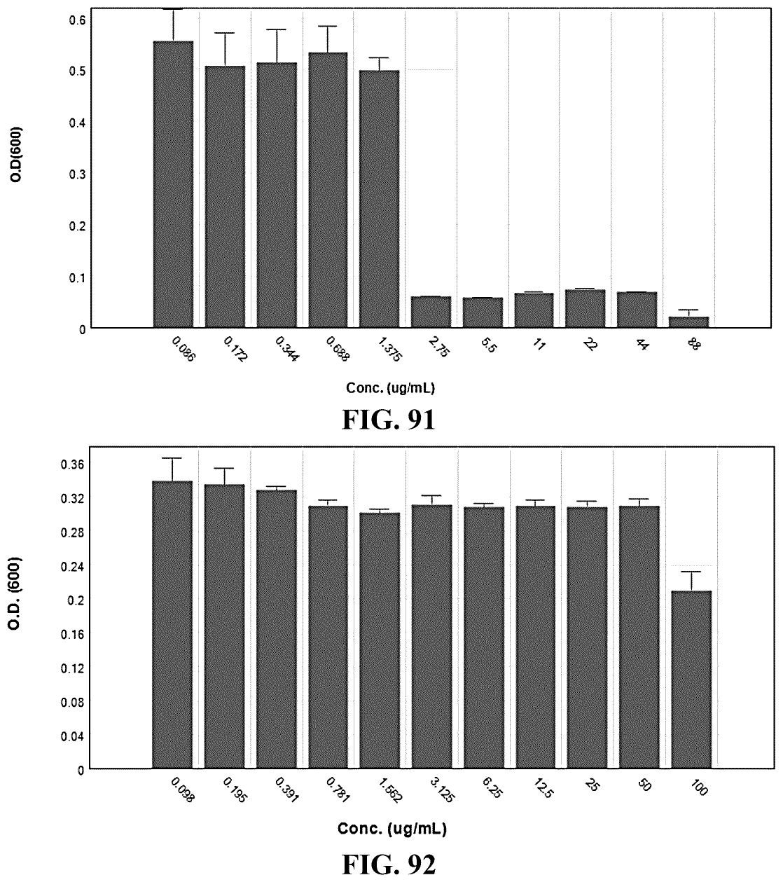

Here we show the MIC graph for GK600 on E. coli (FIG. 89A), S. aureus (FIG. 90) and MRSA (FIG. 91). GK600 shows bacterial growth inhibition at 12.5 μg/mL against E. coli, 3.1 μg/mL against S. aureus, and 2.75 μg/mL against MRSA.

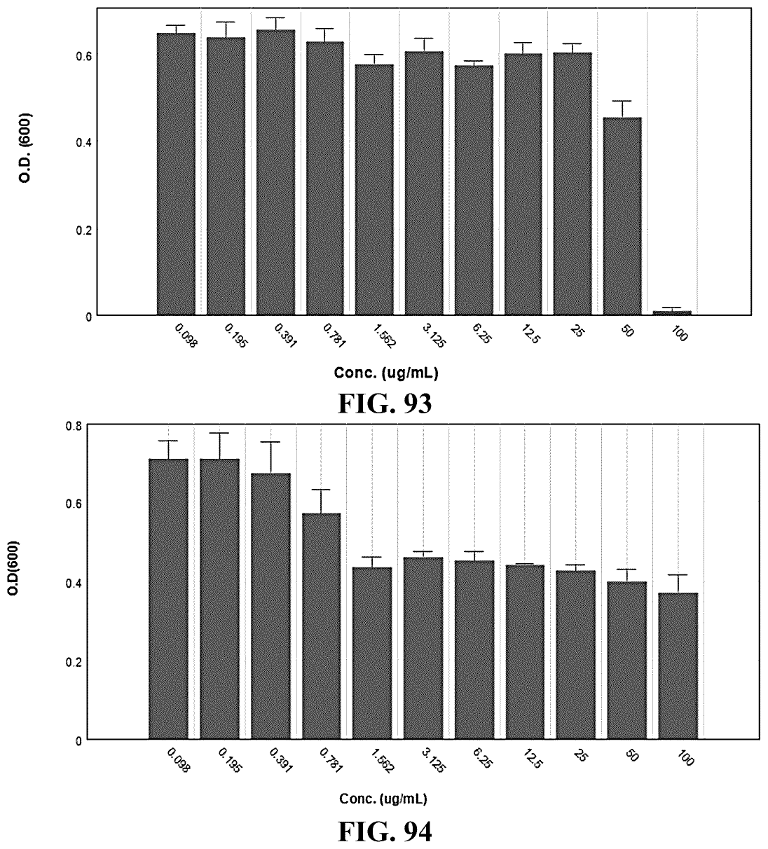

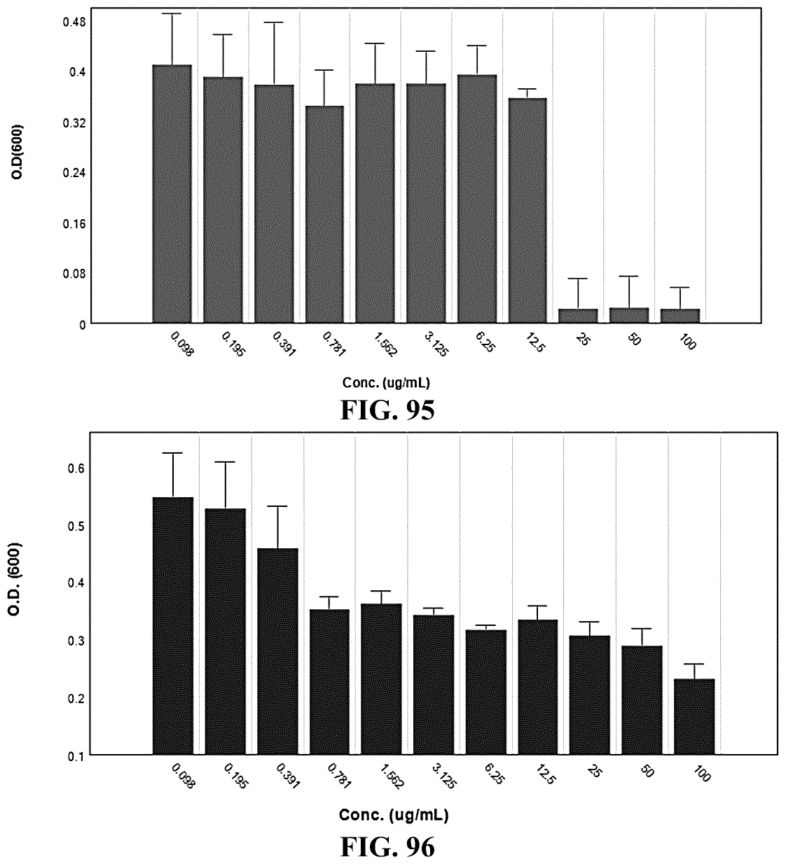

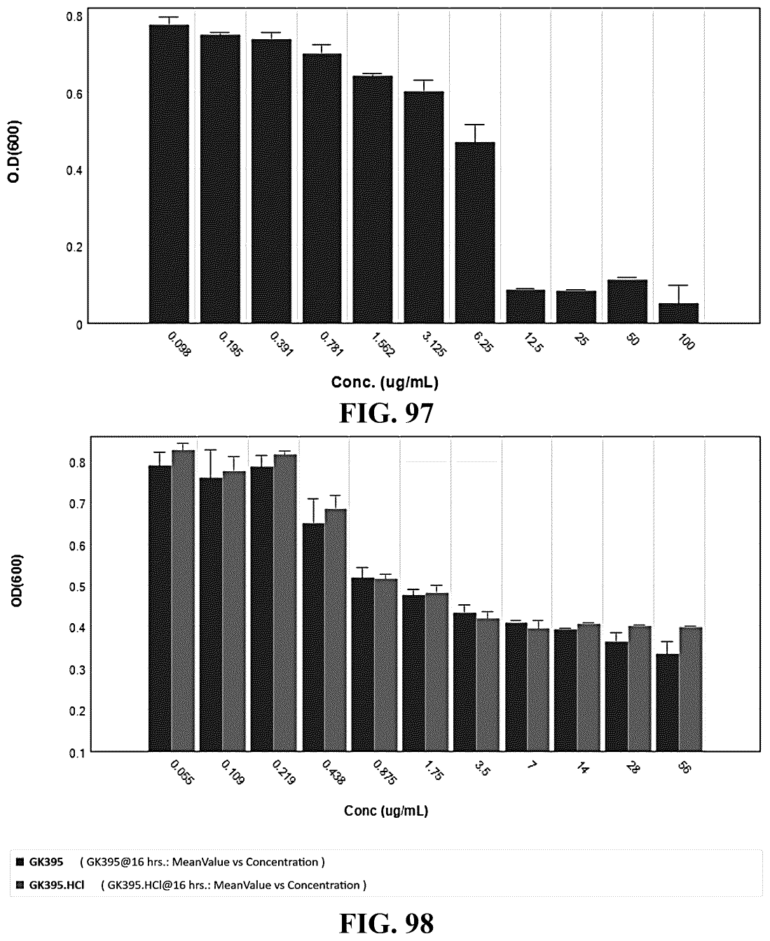

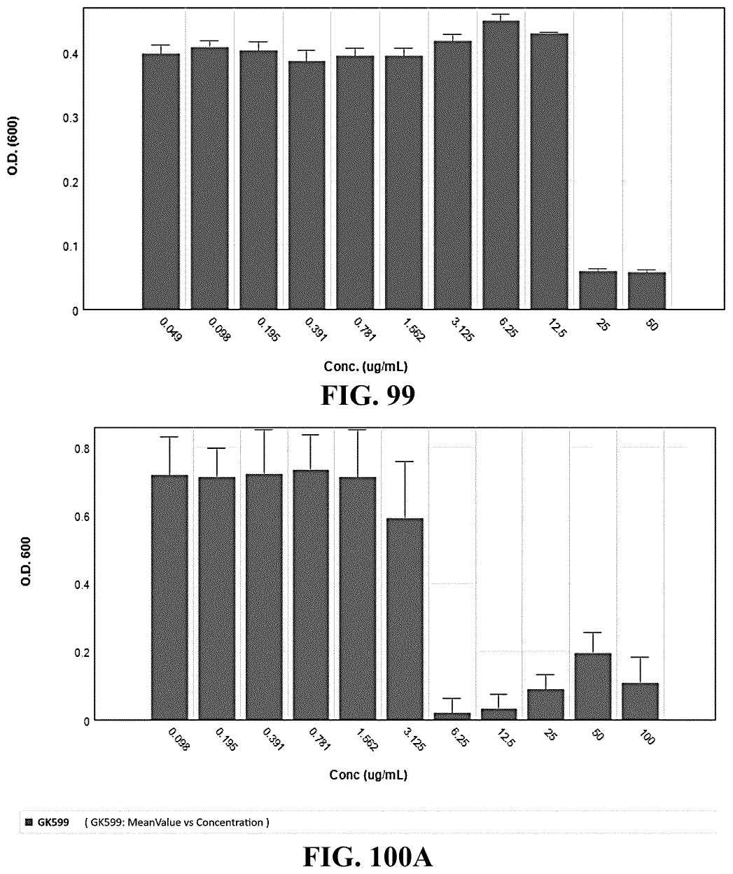

We also tested the activity of harmaline adducts GK317, GK350, GK360, and GK395 again E. coli and S. aureus. GK317 didn't show any activity on E. coli (FIG. 92), however it showed MIC at 100 μg/mL against S. aureus (FIG. 93). GK350 shows ˜50% growth inhibition at 1.5 μg/mL against E. coli (FIG. 94) and MIC at 25 μg/mL against S. aureus (FIG. 95). GK360 shows ˜50% growth inhibition at 0.78 μg/mL against E. coli (FIG. 96) and MIC at 12.5 μg/mL against S. aureus (FIG. 97). GK395 shows ˜50% growth inhibition at 3.5 μg/mL against E. coli (FIG. 98) and MIC at 25 μg/mL against S. aureus (FIG. 99).

GK317, GK350, GK395 are inspired from β-aminobenzoic acid (PABA), which is essential for bacterial cell wall synthesis. We tried to resemble PABA structure to make a potential competitive inhibitor. GK317 has no halogen, GK350 has chlorine, GK395 has bromine and GK360 has carboxylic acid. These adducts, except GK317, were able to inhibit upto 50% growth of E. coli, however harmaine dimer with indole as tether GK580 and GK600 showed MIC of 12.5 μg/mL and 6.25 μg/mL respectively against E. coli and 3.5 μg/mL against S. aureus and was also able to inhibit fungal growth at 12.5 μg/mL. The data also indicated the broad antimicrobial activities of GK580 and GK600 compounds (FIG. 89B).

1. A fused tricyclic compound comprising one β-carboline moiety, the fused tricyclic compound having one of the following structures: 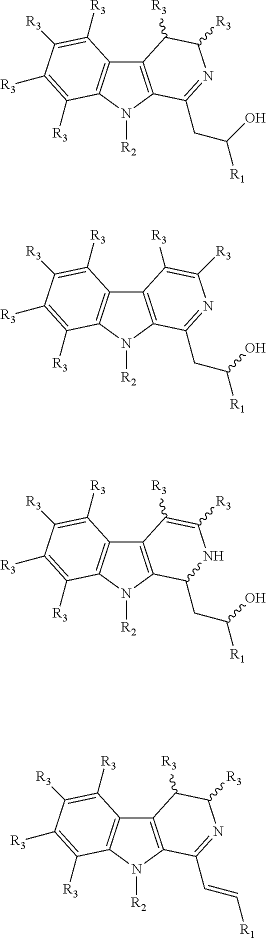 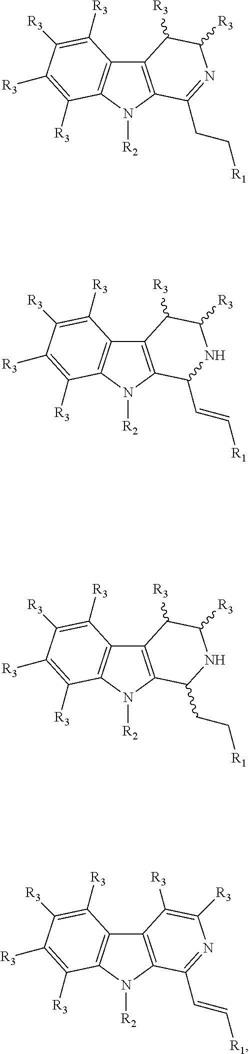
wherein:
each R₁ is independently selected from the group consisting of substituted or unsubstituted aromatic, antiaromatic, or non-aromatic compounds up to 10 atoms, and substituted or unsubstituted fused heterocyclic rings;
each R₂ is independently selected from the group consisting of hydrogen, halogens, sulfonyl chloride, sulphonic acid, sulfonamide, thiol, hydroxy, alkoxy, poly methoxy, H-donor groups, H-bond acceptor groups, aromatic, antiaromatic, or non-aromatic compounds up to 10 atoms, and substituted or unsubstituted fused heterocyclic rings; and
each R₃ is independently selected from the group consisting of hydrogen, oxygen, halogens, sulfonyl chloride, sulphonic acid, thiols, alkyls, nitro groups, hydroxy, alkoxy, H-donor groups, H-bond acceptor groups, aromatic, antiaromatic, or non-aromatic compounds up to 10 atoms, and substituted or unsubstituted fused heterocyclic rings.

2. The compound of claim 1, wherein each R₁ is independently selected from the group consisting of furan, imidazole, benzene, pyridine, indole, indazole, cyclooctatetraene, [10]annulene, pentalene, indene, naphthalene, heptalene, biphenylene, as-indacene, acenaphthylene, fluorene, phenalene, anthracene, pyrene, fluoranthene, imidazopyridines, pyrazopyridines, oxazolopyridines, isooxazolopyridines, cyclopropane, cyclopentane, methyl, ethyl, propyl, isopropyl, pentadiene, hexane, hexene, thioridazine, purines, coumarins, and substituted derivatives thereof having one or more substitutions selected from the group consisting of halogens, C1-C15 alkyls, hydroxy, carboxylic acids, amines, and amides, ethers, esters, aldehydes, and ketones.

3. The compound of claim 1, wherein each R₂ is an aromatic, antiaromatic, or non-aromatic compound up to 10 atoms, or substituted or unsubstituted fused heterocyclic ring, selected from the group consisting of furan, imidazole, benzene, pyridine, indole, indazole, cyclooctatetraene, [10]annulene, pentalene, indene, naphthalene, heptalene, biphenylene, as-indacene, acenaphthylene, fluorene, phenalene, anthracene, pyrene, fluoranthene, imidazopyridines, pyrazopyridines, oxazolopyridines, isooxazolopyridines, cyclopropane, cyclopentane, methyl, ethyl, propyl, isopropyl, pentadiene, hexane, hexene, thioridazine, purines, coumarins, or substituted derivatives thereof having one or more substitutions selected from the group consisting of halogens, C1-C15 alkyls, hydroxy, carboxylic acids, amines, and amides, ethers, esters, aldehydes, and ketones.

4. The compound of claim 1, wherein each R₃ is an aromatic, antiaromatic, or non-aromatic compound up to 10 atoms, or substituted or unsubstituted fused heterocyclic ring, selected from the group consisting of furan, imidazole, benzene, pyridine, indole, indazole, cyclooctatetraene, [10]annulene, pentalene, indene, naphthalene, heptalene, biphenylene, as-indacene, acenaphthylene, fluorene, phenalene, anthracene, pyrene, fluoranthene, imidazopyridines, pyrazopyridines, oxazolopyridines, isooxazolopyridines, cyclopropane, cyclopentane, methyl, ethyl, propyl, isopropyl, pentadiene, hexane, hexene, thioridazine, purines, coumarins, or substituted derivatives thereof having one or more substitutions selected from the group consisting of halogens, C1-C15 alkyls, hydroxy, carboxylic acids, amines, and amides, ethers, esters, aldehydes, and ketones.

5. The compound of claim 1, wherein the β-carboline moiety is harmaline, harmine, or tetrahydro harmine.

6. The compound of claim 1, wherein the compound is one of:

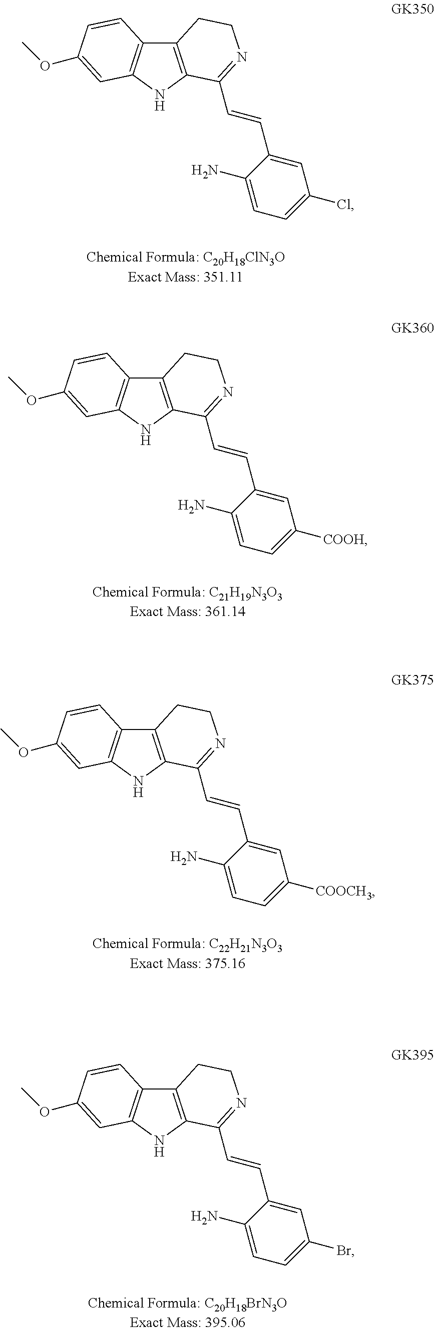 [SMILES: COc1ccc2c3c([nH]c2c1)C(/C=C/c1cc(Cl)ccc1N)=NCC3 ; COc1ccc2c3c([nH]c2c1)C(/C=C/c1cc(C(=O)O)ccc1N)=NCC3 ; COC(=O)c1ccc(N)c(/C=C/C2=NCCc3c2[nH]c2cc(OC)ccc32)c1 ; COc1ccc2c3c([nH]c2c1)C(/C=C/c1cc(Br)ccc1N)=NCC3]

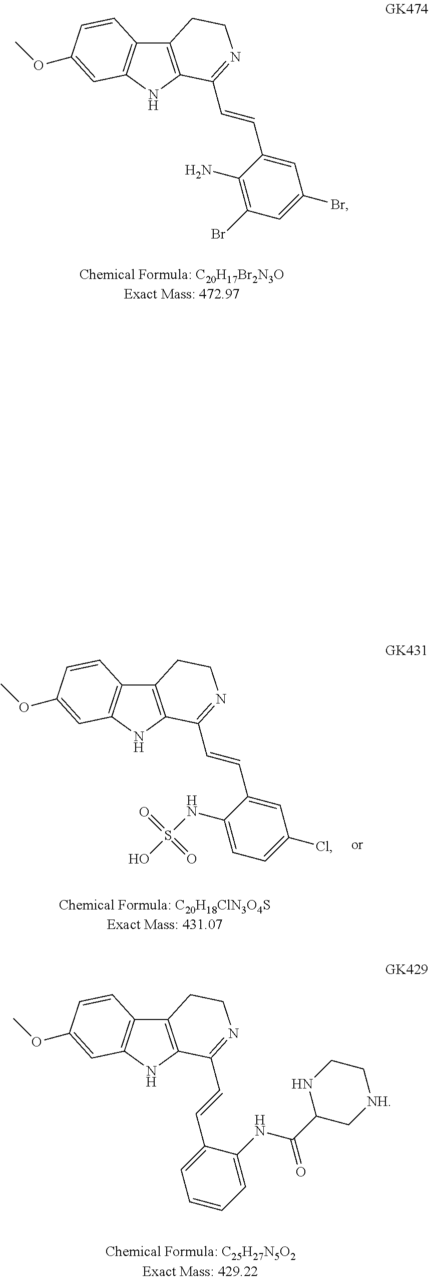 [SMILES: COc1ccc2c3c([nH]c2c1)C(/C=C/c1cc(Br)cc(Br)c1N)=NCC3 ; COc1ccc2c3c([nH]c2c1)C(/C=C/c1cc(Cl)ccc1NS(=O)(=O)O)=NCC3 ; "or" ; COc1ccc2c3c([nH]c2c1)C(/C=C/c1ccccc1NC(=O)C1CNCCN1)=NCC3]

7. The compound of claim 1, wherein the compound further comprises a second β-carboline moiety.

8. The compound of claim 7, wherein the second β-carboline moiety is a harmaline, harmine, or tetrahydro harmine.

9. The compound of claim 7, wherein the compound is a dimer having one of the following structures: 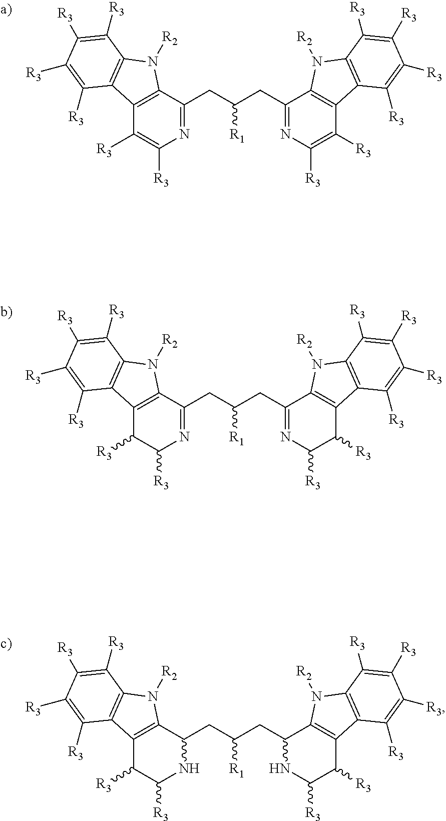
where each R₁, R₂, and R₃ is defined as in claim 1.

10. The compound of claim 1, wherein the compound is a polycyclic compound comprising at least one of said fused tricyclic compounds and having the structure:

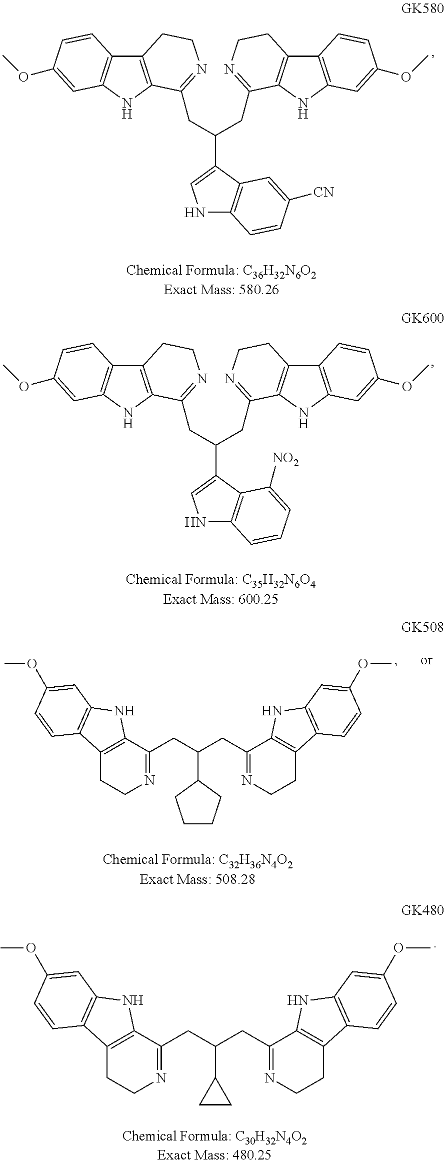

11. A fused tricyclic compound synthesis process comprising reacting a first amount of harmaline with a compound comprising an aldehyde moiety and a solvent to yield a product comprising a tricyclic β-carboline adduct compound,
wherein said reacting takes place in an extraction glassware set up comprising an extractor body having a top opening and a bottom opening, a condenser having a bottom opening configured to receive said top opening of the extractor body, and a flask having a top opening, said top opening of the extractor body being attached to said bottom opening of the condenser and said bottom opening of the extractor body being attached to said top opening of the flask.

12. The method of claim 11, wherein said reacting further comprises reacting a second amount of harmaline with a compound comprising an aldehyde moiety, the solvent, and the product comprising a tricyclic β-carboline adduct compound to yield a product comprising tricyclic β-carboline dimer compound comprising two β-carboline moieties linked via a CH₂ methine group bonded to respective methyl substituents of the β-carboline moieties.

13. A method of inhibiting microbial activity, said method comprising contacting a microbe with a fused tricyclic compound according to claim 1.

14. The method of claim 13, said method comprising administering said compound or a composition comprising said compound to a subject in need thereof.

15. The method of claim 13, wherein said microbe is a bacteria, fungus, or virus.

16. A method for treatment or prophylaxis of a microbial infection, said method comprising administering a compound according to claim 1 or a composition comprising said compound to a subject in need thereof.

17. The method of claim 16, wherein said subject is suffering from a microbial infection before said administering step.

18. The method of claim 16, wherein said subject is at risk of a microbial infection before said administering step.

19. An antimicrobial composition comprising a fused tricyclic compound according to claim 1 dispersed in a pharmaceutically-acceptable carrier.

19. A medicament for inhibiting microbial activity and/or treating a microbial infection, said medicament comprising a fused tricyclic compound according to claim 1 dispersed in a pharmaceutically-acceptable carrier.


SEQUENCE LISTING (6 sequences)

SEQ ID NO: 1 - DNA, 20 bp, synthetic construct
  source 1..20: mol_type = other DNA; note = Forward Primer
gctgaacatgtcaacaactc

SEQ ID NO: 2 - DNA, 20 bp, synthetic construct
  source 1..20: mol_type = other DNA; note = Reverse Primer
gcaatgatggattgactagc

SEQ ID NO: 3 - DNA, 20 bp, synthetic construct
  source 1..20: mol_type = other DNA; note = Probe
actaattctcctcggcgggc

SEQ ID NO: 4 - DNA, 20 bp, synthetic construct
  source 1..20: mol_type = other DNA; note = Forward Primer
tggtcgctctgttaatgaaa

SEQ ID NO: 5 - DNA, 20 bp, synthetic construct
  source 1..20: mol_type = other DNA; note = Reverse Primer
aaatttcttcctagcagctc

SEQ ID NO: 6 - DNA, 20 bp, synthetic construct
  source 1..20: mol_type = other DNA; note = Probe
ccctcctgagaggcaacacc